(19) United States
(12) Patent Application Publication
(10) Pub. No.: US 2018/0342322 A1
(43) Pub. Date: Nov. 29, 2018

(54) METHOD AND SYSTEM FOR CHARACTERIZATION FOR APPENDIX-RELATED CONDITIONS ASSOCIATED WITH MICROORGANISMS

(71) Applicant: uBiome, Inc., San Francisco, CA (US)

(72) Inventors: Zachary Apte, San Francisco, CA (US); Jessica Richman, San Francisco, CA (US); Daniel Almonacid, San Francisco, CA (US); Rodrigo Ortiz, Santiago (CL); Catalina Valdivia, Santiago (CL); Inti Pedroso, Santiago (CL); Paz Tapia, Santiago (CL)

(21) Appl. No.: 16/039,276

(22) Filed: Jul. 18, 2018

Related U.S. Application Data
(63) Continuation-in-part of application No. 15/606,743, filed on May 26, 2017.
Application No. 15/606,743 is a continuation of application No. 14/919,614, filed on Oct. 21, 2015, now Pat. No. 9,703,929.
(60) Provisional application No. 62/066,369, filed on Oct. 21, 2014.
(60) Provisional application No. 62/087,551, filed on Dec. 4, 2014.
(60) Provisional application No. 62/092,999, filed on Dec. 17, 2014.
(60) Provisional application No. 62/147,376, filed on Apr. 14, 2015.
(60) Provisional application No. 62/147,212, filed on Apr. 14, 2015.
(60) Provisional application No. 62/147,362, filed on Apr. 14, 2015.
(60) Provisional application No. 62/146,855, filed on Apr. 13, 2015.
(60) Provisional application No. 62/206,654, filed on Aug. 18, 2015.
(60) Provisional application No. 62/533,816, filed on Jul. 18, 2017.

(51) Int. Cl. G16H 50/20; G16H 10/60; G06F 19/28

(52) U.S. Cl. CPC G16H 50/20; G06F 19/28; G16H 10/60

(57) ABSTRACT

Embodiments of a method and/or system for characterizing one or more appendix-related conditions can include determining a microorganism dataset associated with a set of subjects; and/or performing a characterization process associated with the one or more appendix-related conditions, based on the microorganism dataset, where performing the characterization process can additionally or alternatively include performing an appendix-related characterization process for the one or more appendix-related conditions, and/or determining one or more therapies.

### CROSS-REFERENCE TO RELATED APPLICATIONS

This application is a continuation-in-part of U.S. application Ser. No. 15/606,743, filed 26 May 2017, which is a continuation of U.S. application Ser. No. 14/919,614, filed 21 Oct. 2015, which claims the benefit of U.S. Provisional Application Ser. No. 62/066,369 filed 21 Oct. 2014, U.S. Provisional Application Ser. No. 62/087,551 filed 4 Dec. 2014, U.S. Provisional Application Ser. No. 62/092,999 filed 17 Dec. 2014, U.S. Provisional Application Ser. No. 62/147,376 filed 14 Apr. 2015, U.S. Provisional Application Ser. No. 62/147,212 filed 14 Apr. 2015, U.S. Provisional Application Ser. No. 62/147,362 filed 14 Apr. 2015, U.S. Provisional Application Ser. No. 62/146,855 filed 13 Apr. 2015, and U.S. Provisional Application Ser. No. 62/206,654 filed 18 Aug. 2015, which are each incorporated in its entirety herein by this reference.

This application additionally claims the benefit of U.S. Provisional Application Ser. No. 62/533,816 filed 18 Jul. 2017, which is herein incorporated in its entirety by this reference.

### TECHNICAL FIELD

The disclosure generally relates to genomics and microbiology.

### BACKGROUND

A microbiome can include an ecological community of commensal, symbiotic, and pathogenic microorganisms that are associated with an organism. Characterization of the human microbiome is a complex process. The human microbiome includes over 10 times more microbial cells than human cells, but characterization of the human microbiome is still in nascent stages such as due to limitations in sample processing techniques, genetic analysis techniques, and resources for processing large amounts of data. Present knowledge has clearly established the role of microbiome associations with multiple health conditions, and has become an increasingly appreciated mediator of host genetic and environmental factors on human disease development. The microbiome is suspected to play at least a partial role in a number of health/disease-related states. Further, the microbiome may mediate effects of environmental factors on human, plant, and/or animal health. Given the profound implications of the microbiome in affecting a user's health, efforts related to the characterization of the microbiome, the generation of insights from the characterization, and the generation of therapeutics configured to rectify states of dysbiosis should be pursued. Methods and systems for analyzing the microbiomes of humans and/or providing therapeutic measures based on gained insights have, however, left many questions unanswered.

As such, there is a need in the field of microbiology for a new and useful method and/or system for characterizing, monitoring, diagnosing, and/or intervening in one or more microorganism-related health conditions, such as for individualized and/or population-wide use.

### BRIEF DESCRIPTION OF THE FIGURES

FIGS. 1A-1B are flowchart representations of variations of an embodiment of a method;

FIG. 2 depicts embodiments of a method and system;

FIG. 3 depicts a variation of a process for generation of a characterization model in an embodiment of a method;

FIG. 4 depicts variations of mechanisms by which probiotic-based therapies operate in an embodiment of a method;

FIG. 5 depicts variations of sample processing in an embodiment of a method;

FIG. 6 depicts examples of notification provision;

FIG. 7 depicts a schematic representation of variations of an embodiment of the method;

FIGS. 8A-8B depict variations of performing characterization processes with models;

FIG. 9 depicts promoting a therapy in an embodiment of a method.

### DESCRIPTION OF THE EMBODIMENTS

The following description of the embodiments is not intended to limit the embodiments, but rather to enable any person skilled in the art to make and use.

### 1. Overview

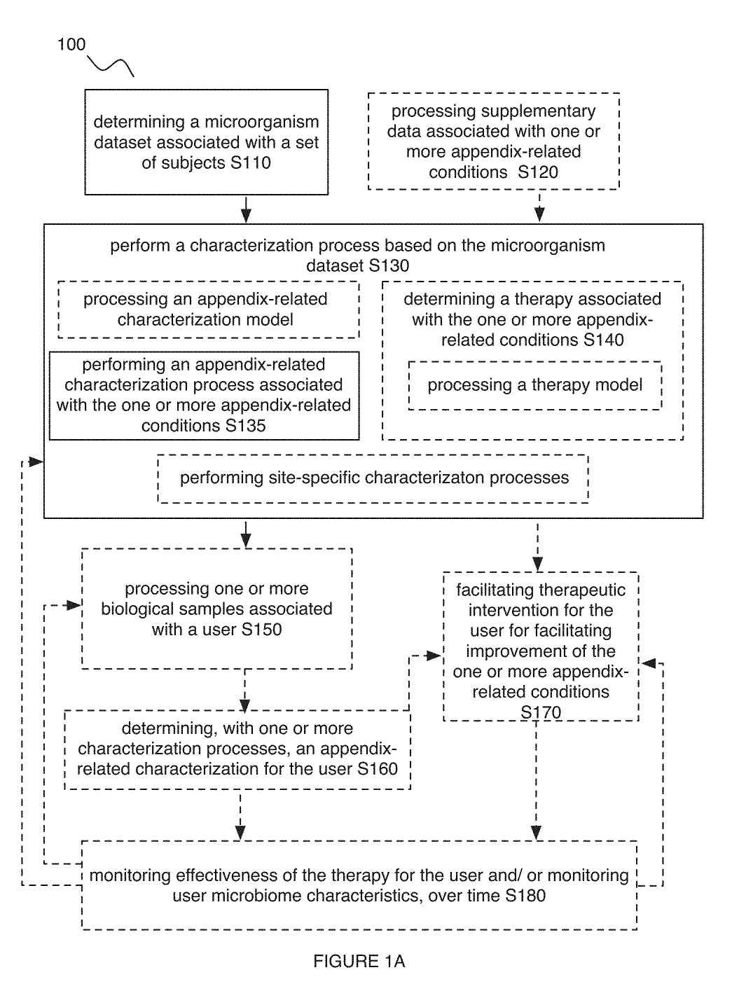

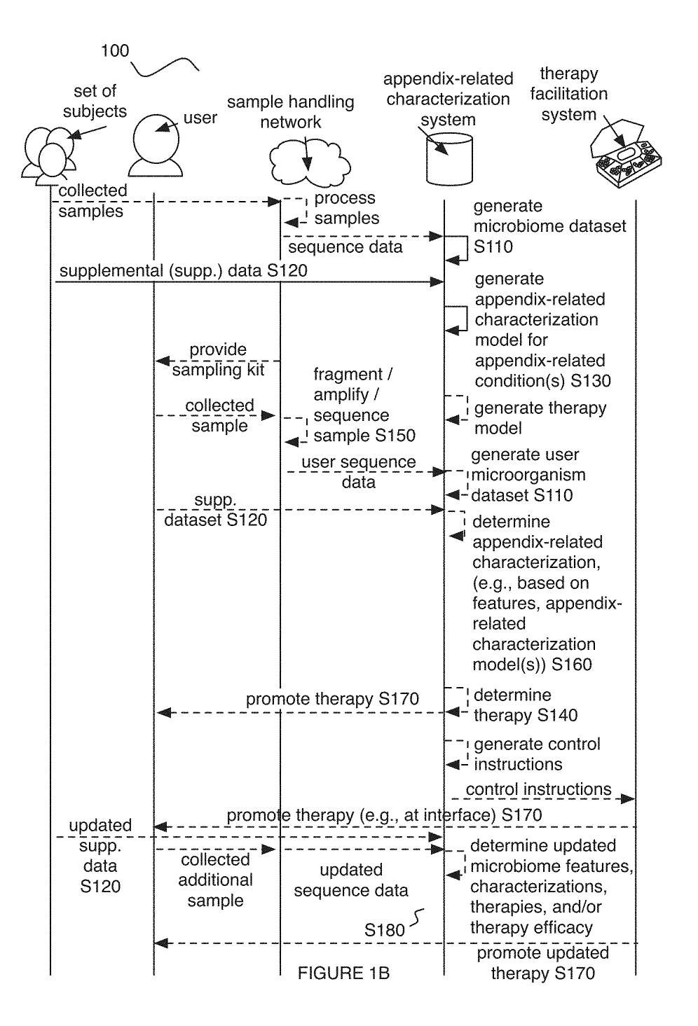

As shown in FIGS. 1A-1B, embodiments of a method 100 for characterizing one or more appendix-related conditions can include: determining a microorganism dataset (e.g., microorganism sequence dataset, microbiome composition diversity dataset such as based upon a microorganism sequence dataset, microbiome functional diversity dataset such as based upon a microorganism sequence dataset, etc.) associated with a set of users (e.g., determining the microorganism dataset based on samples from a set of subjects) Silo; and/or performing a characterization process (e.g., pre-processing, feature determination, feature processing, appendix-related characterization model processing, etc.) associated with the one or more appendix-related conditions, based on the microorganism dataset (e.g., based on microbiome composition features and/or microbiome functional features derived from the microorganism dataset and associated with the one or more appendix-related conditions; etc.) S130, where performing the characterization process can additionally or alternatively include performing an appendix-related characterization process for the one or more appendix-related conditions S135, and/or determining one or more therapies (e.g., determining therapies for preventing, ameliorating, reducing the risk of, and/or otherwise improving the one or more appendix-related conditions, etc.) S140.

Embodiments of the method 100 can additionally or alternatively include one or more of: processing supplementary data associated with (e.g., informative of; describing; indicative of; correlated with, etc.) one or more appendix-related conditions S120; processing one or more biological samples associated with a user (e.g., subject, human, animal, patient; etc.) S150; determining, with one or more characterization processes, an appendix-related characterization for the user for one or more appendix-related conditions, based on a user microorganism dataset (e.g., user microorganism sequence dataset; user microbiome composition dataset; user microbiome function dataset; user microbiome features derived from the user microorganism dataset, where the user microbiome features can correspond to feature values for the microbiome features determined from one or more characterization processes; etc.) associated with a biological sample of the user S160; facilitating therapeutic intervention for the one or more appendix-related conditions for the user (e.g., based upon the appendix-related characterization and/or a therapy model; etc.) S170; monitoring effectiveness of one or more therapies and/or monitoring other suitable components (e.g., microbiome characteristics, etc.) for the user (e.g., based upon processing a series of biological samples from the user), over time (e.g., such as to assess user microbiome characteristics such as user microbiome composition features and/or functional features associated with the therapy, for the user over time, etc.) S180; and/or any other suitable processes.

In a specific example, the method 100 can include determining a microorganism sequence dataset associated with a set of subjects (e.g., including subjects with the appendix-related condition; including subjects without the appendix-related conditions, where samples and/or data associated with such subjects can act as a control; etc.), based on microorganism nucleic acids from samples associated with the set of subjects, where the samples include at least one sample associated with one or more appendix-related conditions; collecting, for the set of subjects, supplementary data associated with one or more appendix-related conditions; determining a set of microbiome features including at least one of a set of microbiome composition features and a set of microbiome functional features, based on the microorganism sequence dataset; generating an appendix-related characterization model based on the supplementary data and the set of microbiome features, where the appendix-related characterization model is associated with the one or more appendix-related conditions; determining an appendix-related characterization for a user for the one or more appendix-related conditions based on the appendix-related characterization model; and facilitating therapeutic intervention for a user for the one or more appendix relation conditions (e.g., providing a therapy to the user for facilitating improvement of the one or more appendix-related conditions, etc.) based on the appendix-related characterization.

In a specific example, the method 100 can include collecting a sample from a user (e.g., via sample kit provision and collection, etc.), where the sample includes microorganism nucleic acids corresponding to the microorganisms associated with one or more appendix-related conditions; determining a microorganism dataset associated with the user based on the microorganism nucleic acids of the sample (e.g., based on sample preparation and/or sequencing with the sample, etc.); determining user microbiome features (e.g., including at least one of user microbiome composition features and user microbiome functional features, etc.) based on the microorganism dataset, where the user microbiome features are associated with the one or more appendix-related conditions; determining an appendix-related characterization for the user for the one or more appendix-related conditions based on the user microbiome features; and/or facilitating therapeutic intervention in relation to a therapy for the user for facilitating improvement of the one or more appendix-related conditions (e.g., promoting the therapy to the user; etc.), based on the appendix-related characterization.

Embodiments of the method 100 and/or system 200 can function to characterize (e.g., assess, evaluate, diagnose, describe, etc.) one or more appendix-related conditions (e.g., characterizing the appendix-related conditions themselves, such as determining microbiome features correlated with and/or otherwise associated with the appendix-related conditions; characterizing one or more appendix-related conditions for one or more users, such as determining propensity metrics for the one or more appendix-related conditions for the one or more users; etc.) and/or one or more users for one or more appendix-related conditions.

Additionally or alternatively, embodiments of the method 100 and/or system 200 can function to identify microbiome features and/or other suitable data associated with (e.g., positive correlated with, negatively correlated with, etc.) one or more appendix-related conditions, such as for use as biomarkers (e.g., for diagnostic processes, for treatment processes, etc.). In examples, appendix-related characterization can be associated with at least one or more of microbiome composition (e.g., microbiome composition diversity, etc.), microbiome function (e.g., microbiome functional diversity, etc.), and/or other suitable microbiome-related aspects. In an example, microorganism features (e.g., describing composition, function, and/or diversity of recognizable patterns, such as in relation to relative abundance of microorganisms that are present in a user's microbiome, such as for subjects exhibiting one or more appendix-related conditions; etc.) and/or microorganism datasets (e.g., from which microbiome features can be derived, etc.) can be used for characterizations (e.g., diagnoses, risk assessments, etc.), therapeutic intervention facilitation, monitoring, and/or other suitable purposes, such as by using bioinformatics pipelines, analytical techniques, and/or other suitable approaches described herein. Additionally or alternatively, embodiments of the method 100 and/or system 200 can function to perform cross-condition analyses for a plurality of appendix-related conditions (e.g., performing characterization processes for a plurality of appendix-related conditions, such as determining correlation, covariance, comorbidity, and/or other suitable relationships between different appendix-related conditions, etc.), such as in the context of characterizing (e.g., diagnosing; providing information related to; etc.) and/or treating a user.

Additionally or alternatively, embodiments can function to facilitate therapeutic intervention (e.g., therapy selection; therapy promotion and/or provision; therapy monitoring; therapy evaluation; etc.) for one or more appendix-related conditions, such as through promotion of associated therapies (e.g., in relation to specific body sites such as a gut site, skin site, nose site, mouth site, genital site, other suitable body sites, other collection sites; therapies determined by therapy models; etc.). Additionally or alternatively, embodiments can function to generate models (e.g., appendix-related characterization models such as for phenotypic prediction; therapy models such as for therapy determination; machine learning models such as for feature processing; etc.), such as models that can be used to characterize and/or diagnose users based on their microbiome (e.g., user microbiome features; as a clinical diagnostic; as a companion diagnostic, etc.), and/or that can be used to select and/or provide therapies for subjects in relation to one or more appendix-related conditions. Additionally or alternatively, embodiments can perform any suitable functionality described herein.

As such, data from populations of users (e.g., populations of subjects associated with one or more appendix-related conditions; positively or negatively correlated with one or more appendix-related conditions; etc.) can be used to characterize subsequent users, such as for indicating microorganism-related states of health and/or areas of improvement, and/or to facilitate therapeutic intervention (e.g., promoting one or more therapies; facilitating modulation of the composition and/or functional diversity of a user's microbiome toward one or more of a set of desired equilibrium states, such as states correlated with improved health states associated with one or more appendix-related conditions; etc.), such as in relation to one or more appendix-related conditions. Variations of the method 100 can further facilitate selection, monitoring (e.g., efficacy monitoring, etc.) and/or adjusting of therapies provided to a user, such as through collection and analysis (e.g., with appendix-related characterization models) of additional samples from a user over time (e.g., throughout the course of a therapy regimen, through the extent of a user's experiences with appendix-related conditions; etc.), across body sites (e.g., across sample collection sites of a user, such as collection sites corresponding to a particular body site type such as a gut site, mouth site, nose site, skin site, genital site; etc.), in addition or alternative to processing supplementary data over time, such as for one or more appendix-related conditions. However, data from populations, subgroups, individuals, and/or other suitable entities can be used by any suitable portions of embodiments of the method 100 and/or system 200 for any suitable purpose.

Embodiments of the method 100 and/or system 200 can preferably determine and/or promote (e.g., provide; present; notify regarding; etc.) characterizations and/or therapies for one or more appendix-related conditions, and/or any suitable portions of embodiments of the method 100 and/or system 200 can be performed in relation to appendix-related conditions. Appendix-related conditions can include any one or more of: appendicitis (e.g., acute appendicitis; suspected appendicitis; etc.), appendix inflammation, appendix cancer (e.g., appendiceal carcinoma), carcinoid tumors, carcinoid syndrome, fecalith, ovarian mucinous tumor, Crohn's disease (e.g., of the appendix), lymphoid hyperplasia, congenital abnormalities (e.g., congenital absence; appendiceal duplication; etc.), endometriosis of the appendix, peritoneal endosalpingiosis, vasculitis (e.g., of the appendix, etc.), neural proliferations (e.g., of the appendix, etc.) mesenchymal tumors, nonmyogenic neoplasms, lymphoma, irritable bowel syndrome, mononucleosis, measles, gastrointestinal infections, intussusception, adenoma, diverticular disease, gut immunity-related conditions, infection; comorbid conditions, and/or any other suitable conditions associated with appendix.

Additionally or alternatively, appendix-related conditions can include one or more of: diseases, symptoms (e.g., blood flow prevention; tissue death; excess cell production; dull pain proximal the appendix; appetite loss; tissue bursting; pain; appendix swelling; abdominal swelling; inability to pass gas; painful urination; sharp pain; cramps; nausea; vomiting; fever; rebound tenderness; swollen body regions such as abdomen; back pain; constipation; diarrhea; peritonitis; abscessing; organ failure; muscle guarding; obstipation; scar tissue; etc.), causes (e.g., triggers; impacted fecal matter; lymphoid hyperplasia; obstruction such as due to stool, parasites, growths; abdomen trauma; etc.), disorders, associated risk (e.g., propensity scores, etc.), associated severity, behaviors (e.g., physical activity behavior; alcohol consumption; smoking behaviors; stress-related characteristics; other psychological characteristics; sickness; social behaviors; caffeine consumption; alcohol consumption; sleep habits; other habits; diet-related behaviors such as fiber intake, fruit intake, vegetable intake; meditation and/or other relaxation behaviors; lifestyle conditions associated with appendix-related conditions; lifestyle conditions informative of, correlated with, indicative of, facilitative of, and/or otherwise associated with diagnosis and/or therapeutic intervention for appendix-related conditions; behaviors affecting and/or otherwise associated with the appendix and/or appendix-related conditions; etc.), environmental factors, demographic-related characteristics (e.g., age, weight, race, gender, etc.), phenotypes (e.g., phenotypes measurable for a human, animal, plant, fungi body; phenotypes associated with appendix and/or other related aspects, etc.), and/or any other suitable aspects associated with appendix-related conditions. In an example, one or more appendix-related conditions can interfere with normal physical, mental, social and/or emotional function. In examples, one or more appendix-related conditions can be characterized by and/or diagnosed by computed tomography (CT scan), ultrasound, colonoscopy, biopsy, blood test, abdominal exam (e.g., to detect inflammation, etc.), urine test (e.g., to detect infection; etc.), diagnostic imaging, medical interview, medical history, survey, sensor data, and/or through any suitable techniques (e.g., techniques available for diagnosis for appendix-related conditions, etc.).

Embodiments of the method 100 and/or system 200 can be implemented for a single user, such as in relation to applying one or more sample handling processes and/or characterization processes for processing one or more biological samples (e.g., collected across one or more collection sites, etc.) from the user, for appendix-related characterization, facilitating therapeutic intervention, and/or for any other suitable purpose. Additionally or alternatively, embodiments can be implemented for a population of subjects (e.g., including the user, excluding the user), where the population of subjects can include subjects similar to and/or dissimilar to any other subjects for any suitable type of characteristics (e.g., in relation to appendix-related conditions, demographic characteristics, behaviors, microbiome composition and/or function, etc.); implemented for a subgroup of users (e.g., sharing characteristics, such as characteristics affecting appendix-related characterization and/or therapy determination; etc.); implemented for plants, animals, microorganisms, and/or any other suitable entities. Thus, information derived from a set of subjects (e.g., population of subjects, set of subjects, subgroup of users, etc.) can be used to provide additional insight for subsequent users. In a variation, an aggregate set of biological samples is preferably associated with and processed for a wide variety of subjects, such as including subjects of one or more of: different demographic characteristics (e.g., genders, ages, marital statuses, ethnicities, nationalities, socioeconomic statuses, sexual orientations, etc.), different appendix-related conditions (e.g., health and disease states; different genetic dispositions; etc.), different living situations (e.g., living alone, living with pets, living with a significant other, living with children, etc.), different dietary habits (e.g., omnivorous, vegetarian, vegan, sugar consumption, acid consumption, caffeine consumption, etc.), different behavioral tendencies (e.g., levels of physical activity, drug use, alcohol use, etc.), different levels of mobility (e.g., related to distance traveled within a given time period), and/or any other suitable characteristic (e.g., characteristics influencing, correlated with, and/or otherwise associated with microbiome composition and/or function, etc.). In examples, as the number of subjects increases, the predictive power of processes implemented in portions of embodiments of the method 100 and/or system 200 can increase, such as in relation to characterizing subsequent users (e.g., with varying characteristics, etc.) based upon their microbiomes (e.g., in relation to different collection sites for samples for the users, etc.). However, portions of embodiments of the method 100 and/or system 200 can be performed and/or configured in any suitable manner for any suitable entity or entities.

Data described herein (e.g., microbiome features, microorganism datasets, models, appendix-related characterizations, supplementary data, notifications, etc.) can be associated with any suitable temporal indicators (e.g., seconds, minutes, hours, days, weeks, etc.) including one or more: temporal indicators indicating when the data was collected (e.g., temporal indicators indicating when a sample was collected; etc.), determined, transmitted, received, and/or otherwise processed; temporal indicators providing context to content described by the data (e.g., temporal indicators associated with appendix-related characterizations, such as where the appendix-related characterization describes the appendix-related conditions and/or user microbiome status at a particular time; etc.); changes in temporal indicators (e.g., changes in appendix-related characterizations over time, such as in response to receiving a therapy; latency between sample collection, sample analysis, provision of an appendix-related characterization or therapy to a user, and/or other suitable portions of embodiments of the method 100; etc.); and/or any other suitable indicators related to time.

Additionally or alternatively, parameters, metrics, inputs, outputs, and/or other suitable data can be associated with value types including: scores (e.g., appendix-related condition propensity scores; feature relevance scores; correlation scores, covariance scores, microbiome diversity scores, severity scores; etc.), individual values (e.g., individual appendix-related condition scores, such as condition propensity scores, for different collection sites, etc.), aggregate values, (e.g., overall scores based on individual microorganism-related scores for different collection sites, etc.), binary values (e.g., presence or absence of a microbiome feature; presence or absence of an appendix-related condition; etc.), relative values (e.g., relative taxonomic group abundance, relative microbiome function abundance, relative feature abundance, etc.), classifications (e.g., appendix-related condition classifications and/or diagnoses for users; feature classifications; behavior classifications; demographic characteristic classifications; etc.), confidence levels (e.g., associated with microorganism sequence datasets; with microbiome diversity scores; with other appendix-related characterizations; with other outputs; etc.), identifiers, values along a spectrum, and/or any other suitable types of values. Any suitable types of data described herein can be used as inputs (e.g., for different analytical techniques, models, and/or other suitable components described herein), generated as outputs (e.g., of different analytical techniques, models, etc.), and/or manipulated in any suitable manner for any suitable components associated with the method 100 and/or system 200.

One or more instances and/or portions of embodiments of the method 100 and/or processes described herein can be performed asynchronously (e.g., sequentially), concurrently (e.g., parallel data processing; concurrent cross-condition analysis; multiplex sample processing, such as multiplex amplification of microorganism nucleic acid fragments corresponding to target sequences associated with appendix-related conditions; performing sample processing and analysis for substantially concurrently evaluating a panel of appendix-related conditions; computationally determining microorganism datasets, microbiome features, and/or characterizing appendix-related conditions in parallel for a plurality of users; such as concurrently on different threads for parallel computing to improve system processing ability; etc.), in temporal relation (e.g., substantially concurrently with, in response to, serially, prior to, subsequent to, etc.) to a trigger event (e.g., performance of a portion of the method 100), and/or in any other suitable order at any suitable time and frequency by and/or using one or more instances of the system 200, components, and/or entities described herein. In an example, the method 100 can include generating a microorganism dataset based on processing microorganism nucleic acids of one or more biological samples with a bridge amplification substrate of a next generation sequencing platform (and/or other suitable sequencing system) of a sample handling system, and determining microbiome features and microbiome functional diversity features at computing devices operable to communicate with the next generation sequencing platform. However, the method 100 and/or system 200 can be configured in any suitable manner.

### 2. Examples

Microbiome analysis can enable accurate and/or efficient characterization and/or therapy provision (e.g., according to portions of embodiments of the method 100, etc.) for appendix-related conditions caused by, correlated with, and/or otherwise associated with microorganisms. Specific examples of the technology can overcome several challenges faced by conventional approaches in characterizing an appendix-related conditions and/or facilitating therapeutic intervention. First, conventional approaches can require patients to visit one or more care providers to receive a characterization and/or a therapy recommendation for an appendix-related condition (e.g., through diagnostic medical procedures), which can amount to inefficiencies and/or health-risks associated with the amount of time elapsed before diagnosis and/or treatment, with inconsistency in healthcare quality, and/or with other aspects of care provider visitation. Second, conventional genetic sequencing and analysis technologies for human genome sequencing can be incompatible and/or inefficient when applied to the microbiome (e.g., where the human microbiome can include over 10 times more microbial cells than human cells; where viable analytical techniques and the means of leveraging the analytical techniques can differ; where optimal sample processing techniques can differ, such as for reducing amplification bias; where different approaches to appendix-related characterizations can be employed; where the types of conditions and correlations can differ; where causes of the associated conditions and/or viable therapies for the associated conditions can differ; where sequence reference databases can differ; where the microbiome can vary across different body regions of the user such as at different collection sites; etc.). Third, the onset of sequencing technologies (e.g., next-generation sequencing, associated technologies, etc.) has given rise to technological issues (e.g., data processing and analysis issues for the plethora of generated sequence data; issues with processing a plurality of biological samples in a multiplex manner; information display issues; therapy prediction issues; therapy provision issues, etc.) that would not exist but for the unprecedented advances in speed and data generation associated with sequencing genetic material. Specific examples of the method 100 and/or system 200 can confer technologically-rooted solutions to at least the challenges described above.

First, specific examples of the technology can transform entities (e.g., users, biological samples, therapy facilitation systems including medical devices, etc.) into different states or things. For example, the technology can transform a biological sample into components able to be sequenced and analyzed to generate microorganism dataset and/or microbiome features usable for characterizing users in relation to one or more appendix-related conditions (e.g., such as through use of next-generation sequencing systems, multiplex amplification operations; etc.). In another example, the technology can identify, discourage and/or promote (e.g., present, recommend, provide, administer, etc.), therapies (e.g., personalized therapies based on an appendix-related characterization; etc.) and/or otherwise facilitate therapeutic intervention (e.g., facilitating modification of a user's microbiome composition, microbiome functionality, etc.), which can prevent and/or ameliorate one or more appendix-related conditions, such as thereby transforming the microbiome and/or health of the patient (e.g., improving a health state associated with an appendix-related condition; etc.), such as applying one or more microbiome features (e.g., applying correlations, relationships, and/or other suitable associations between microbiome features and one or more appendix-related conditions; etc.). In another example, the technology can transform microbiome composition and/or function at one or more different body sites of a user (e.g., one or more different collection sites, etc.), such as targeting and/or transforming microorganisms associated with a gut, nose, skin, mouth, and/or genitals microbiome (e.g., by facilitating therapeutic intervention in relation to one or more site-specific therapies; etc.). In another example, the technology can control therapy facilitation systems (e.g., dietary systems; automated medication dispensers; behavior modification systems; diagnostic systems; disease therapy facilitation systems; etc.) to promote therapies (e.g., by generating control instructions for the therapy facilitation system to execute; etc.), thereby transforming the therapy facilitation system.

Second, specific examples of the technology can confer improvements in computer-related technology (e.g., improving computational efficiency in storing, retrieving, and/or processing microorganism-related data for appendix-related conditions; computational processing associated with biological sample processing, etc.) such as by facilitating computer performance of functions not previously performable. For example, the technology can apply a set of analytical techniques in a non-generic manner to non-generic microorganism datasets and/or microbiome features (e.g., that are recently able to be generated and/or are viable due to advances in sample processing techniques and/or sequencing technology, etc.) for improving appendix-related characterizations and/or facilitating therapeutic intervention for appendix-related conditions.

Third, specific examples of the technology can confer improvements in processing speed, appendix-related characterization, accuracy, microbiome-related therapy determination and promotion, and/or other suitable aspects in relation to appendix-related conditions. For example, the technology can leverage non-generic microorganism datasets to determine, select, and/or otherwise process microbiome features of particular relevance to one or more appendix-related conditions (e.g., processed microbiome features relevant to an appendix-related condition; cross-condition microbiome features with relevance to a plurality of appendix-related conditions, etc.), which can facilitate improvements in accuracy (e.g., by using the most relevant microbiome features; by leveraging tailored analytical techniques; etc.), processing speed (e.g., by selecting a subset of relevant microbiome features; by performing dimensionality reduction techniques; by leveraging tailored analytical techniques; etc.), and/or other computational improvements in relation to phenotypic prediction (e.g., indications of the appendix-related conditions, etc.), other suitable characterizations, therapeutic intervention facilitation, and/or other suitable purposes. In a specific example, the technology can apply feature-selection rules (e.g., microbiome feature-selection rules for composition, function; for supplemental features extracted from supplementary datasets; etc.) to select an optimized subset of features (e.g., microbiome functional features relevant to one or more appendix-related conditions; microbiome composition diversity features such as reference relative abundance features indicative of healthy, presence, absence, and/or other suitable ranges of taxonomic groups associated with appendix-related conditions; user relative abundance features that can be compared to reference relative abundance features correlated with appendix-related conditions and/or therapy responses; etc.) out of a vast potential pool of features (e.g., extractable from the plethora of microbiome data such as sequence data; identifiable by univariate statistical tests; etc.) for generating, applying, and/or otherwise facilitating characterization and/or therapies (e.g., through models, etc.). The potential size of microbiomes (e.g., human microbiomes, animal microbiomes, etc.) can translate into a plethora of data, giving rise to questions of how to process and analyze the vast array of data to generate actionable microbiome insights in relation to appendix-related conditions. However, the feature-selection rules and/or other suitable computer-implementable rules can enable one or more of: shorter generation and execution times (e.g., for generating and/or applying models; for determining appendix-related characterizations and/or associated therapies; etc.); optimized sample processing techniques (e.g., improving transformation of microorganism nucleic acids from biological samples through using primer types, other biomolecules, and/or other sample processing components identified through computational analysis of taxonomic groups, sequences, and/or other suitable data associated with appendix-related conditions, such as while optimizing for improving specificity, reducing amplification bias, and/or other suitable parameters; etc.); model simplification facilitating efficient interpretation of results; reduction in overfitting; network effects associated with generating, storing, and applying appendix-related characterizations for a plurality of users over time in relation to appendix-related conditions (e.g., through collecting and processing an increasing amount of microbiome-related data associated with an increasing number of users to improve predictive power of the appendix-related characterizations and/or therapy determinations; etc.); improvements in data storage and retrieval (e.g., storing and/or retrieving appendix-related characterization models; storing specific models such as in association with different users and/or sets of users, with different appendix-related conditions; storing microorganism datasets in association with user accounts; storing therapy monitoring data in association with one or more therapies and/or users receiving the therapies; storing features, appendix-related characterizations, and/or other suitable data in association with a user, set of users, and/or other entities to improve delivery of personalized characterizations and/or treatments for the appendix-related conditions, etc.), and/or other suitable improvements to technological areas.

Fourth, specific examples of the technology can amount to an inventive distribution of functionality across components including a sample handling system, an appendix-related characterization system, and a plurality of users, where the sample handling system can handle substantially concurrent processing of biological samples (e.g., in a multiplex manner) from the plurality of users, which can be leveraged by the appendix-related characterization system in generating personalized characterizations and/or therapies (e.g., customized to the user's microbiome such as in relation to the user's dietary behavior, probiotics-associated behavior, medical history, demographic characteristics, other behaviors, preferences, etc.) for appendix-related conditions.

Fifth, specific examples of the technology can improve the technical fields of at least genomics, microbiology, microbiome-related computation, diagnostics, therapeutics, microbiome-related digital medicine, digital medicine generally, modeling, and/or other relevant fields. In an example, the technology can model and/or characterize different appendix-related conditions, such as through computational identification of relevant microorganism features (e.g., which can act as biomarkers to be used in diagnoses, facilitating therapeutic intervention, etc.) for appendix-related conditions. In another example, the technology can perform cross-condition analysis to identify and evaluate cross-condition microbiome features associated with (e.g., shared across, correlated across, etc.) a plurality of an appendix-related conditions (e.g., diseases, phenotypes, etc.). Such identification and characterization of microbiome features can facilitate improved health care practices (e.g., at the population and individual level, such as by facilitating diagnosis and therapeutic intervention, etc.), by reducing risk and prevalence of comorbid and/or multi-morbid appendix-related conditions (e.g., which can be associated with environmental factors, and thereby associated with the microbiome, etc.). In specific examples, the technology can apply unconventional processes (e.g., sample processing processes; computational analysis processes; etc.), such as to confer improvements in technical fields.

Sixth, the technology can leverage specialized computing devices (e.g., devices associated with the sample handling system, such as next-generation sequencing systems; appendix-related characterization systems; therapy facilitation systems; etc.) in performing suitable portions associated with embodiments of the method 100 and/or system 200.

Specific examples of the technology can, however, provide any suitable improvements in the context of using non-generalized components and/or suitable components of embodiments of the system 200 for appendix-related characterization, microbiome modulation, and/or for performing suitable portions of embodiments of the method 100.

### 3. System

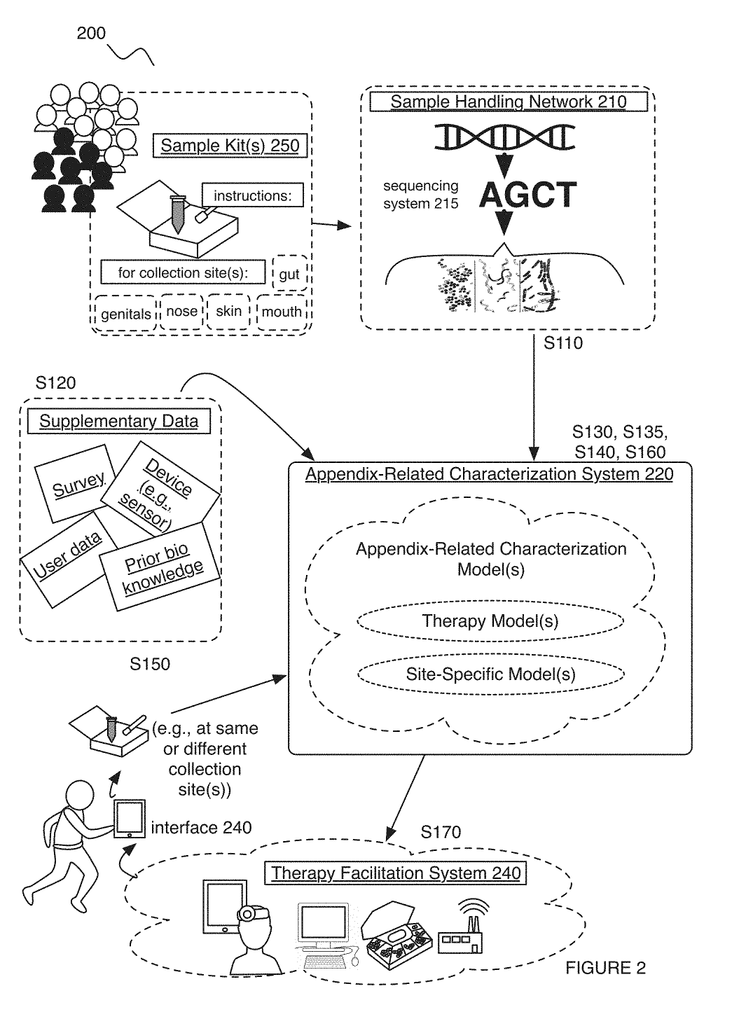

As shown in FIG. 2, embodiments of the system 200 (e.g., for characterizing an appendix-related condition) can include any one or more of: a handling system (e.g., a sample handling system, etc.) 210 operable to collect and/or process biological samples (e.g., collected by users and included in containers including pre-processing reagents; etc.) from one or more users (e.g., a human subject, patient, animal subject, environmental ecosystem, care provider, etc.) for facilitating determination of a microorganism dataset (e.g., microorganism genetic sequences; microorganism sequence dataset; etc.); an appendix-related characterization system 220 operable to determine microbiome features (e.g., microbiome composition features; microbiome functional features; diversity features; relative abundance ranges; such as based on a microorganism dataset and/or other suitable data; etc.), determine appendix-related characterizations (e.g., appendix-related condition characterizations, therapy-related characterizations, characterizations for users, etc.); and/or a therapy facilitation system 230 operable to facilitate therapeutic intervention (e.g., promote a therapy, etc.) for one or more appendix-related conditions (e.g., based on one or more appendix-related conditions; for improving one or more appendix-related conditions; etc.).

Embodiments of the system 200 can include one or more handling systems 210, which can function to receive and/or process (e.g., fragment, amplify, sequence, generate associated datasets, etc.) biological samples to transform microorganism nucleic acids and/or other components of the biological samples into data (e.g., genetic sequences that can be subsequently aligned and analyzed; microorganism datasets; etc.) for facilitating generation of appendix-related characterizations and/or therapeutic intervention. The handling system 210 can additionally or alternatively function to provide sample kits 250 (e.g., including sample containers, instructions for collecting samples from one or more collection sites, etc.) to a plurality of users (e.g., in response to a purchase order for a sample kit 250), such as through a mail delivery system. The handling system 210 can include one or more sequencing systems 215 (e.g., next-generation sequencing systems, sequencing systems for targeted amplicon sequencing, metatranscriptomic sequencing, metagenomic sequencing, sequencing-by-synthesis techniques, capillary sequencing technique, Sanger sequencing, pyrosequencing techniques, nanopore sequencing techniques, etc.) for sequencing one or more biological samples (e.g., sequencing microorganism nucleic acids from the biological samples, etc.), such as in generating microorganism data (e.g., microorganism sequence data, other data for microorganism datasets, etc.). Next-generation sequencing systems (e.g., next-generation sequencing platforms, etc.) can include any suitable sequencing systems (e.g., sequencing platforms, etc.) for one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), amplicon-associated sequencing (e.g., targeted amplicon sequencing), metagenome-associated sequencing (e.g., metatranscriptomic sequencing, metagenomic sequencing, etc.), sequencing-by-synthesis, tunnelling currents sequencing, sequencing by hybridization, mass spectrometry sequencing, microscopy-based techniques, and/or any suitable next-generation sequencing technologies. Additionally or alternatively, sequencing systems 215 can implement any one or more of capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

The handling system 210 can additionally or alternatively include a library preparation system operable to automatically prepare biological samples (e.g., fragment and amplify using primers compatible with genetic targets associated with the appendix-related condition) in a multiplex manner to be sequenced by a sequencing system; and/or any suitable components. The handling system 210 can perform any suitable sample processing techniques described herein. However, the handling system 210 and associated components can be configured in any suitable manner.

Embodiments of the system 200 can include one or more appendix-related characterization systems 220, which can function to determine, analyze, characterize, and/or otherwise process microorganism datasets (e.g., based on processed biological samples leading to microorganism genetic sequences; alignments to reference sequences; etc.), microbiome features (e.g., individual variables; groups of variables; features relevant for phenotypic prediction, for statistical description; variables associated with a sample obtained from an individual; variables associated with appendix-related conditions; variables describing fully or partially, in relative or absolute quantities the sample's microbiome composition and/or functionality; etc.), models, and/or other suitable data for facilitating appendix-related characterization and/or therapeutic intervention. In examples, the appendix-related characterization system 220 can identify data associated with the information of the features that statistically describe the differences between samples associated with one or more appendix-related conditions (e.g., samples associated with presence, absence, risk of, propensity for, and/or other aspects related to appendix-related conditions etc.), such as where the differing analyses can provide complementing views into the features differentiating the different samples (e.g., differentiating the subgroups associated with presence or absence of a condition, etc.). In a specific example, individual predictors, a specific biological process, and/or statistically inferred latent variables can provide complementary information at different levels of data complexity to facilitate varied downstream opportunities in relation to characterization, diagnosis, and/or treatment. In another specific example, the appendix-related characterization system 220 process supplementary data for performing one or more characterization processes.

The appendix-related characterization system 220 can include, generate, apply, and/or otherwise process appendix-related characterization models, which can include any one or more of appendix-related condition models for characterizing one or more appendix-related conditions (e.g., determining propensity of one or more appendix-related conditions for one or more users, etc.), therapy models for determining therapies, and/or any other suitable models for any suitable purposes associated with the embodiments of the system 200 and/or method 100. In a specific example, the appendix-related characterization system 220 can generate and/or apply a therapy model (e.g., based on cross-condition analyses, etc.) for identifying and/or characterizing a therapy used to treat one or more appendix-related conditions. Different appendix-related characterization models (e.g., different combinations of appendix-related characterization models; different models applying different analytical techniques; different inputs and/or output types; applied in different manners such as in relation to time and/or frequency; etc.) can be applied (e.g., executed, selected, retrieves, stored, etc.) based on one or more of: appendix-related conditions (e.g., using different appendix-related characterization models depending on the appendix-related condition or conditions being characterized, such as where different appendix-related characterization models possess differing levels of suitability for processing data in relation to different appendix-related conditions and/or combinations of conditions, etc.), users (e.g., different appendix-related characterization models based on different user data and/or characteristics, demographic characteristics, genetics, environmental factors, etc.), appendix-related characterizations (e.g., different appendix-related characterization models for different types of characterizations, such as a therapy-related characterization versus a diagnosis-related characterization, such as for identifying relevant microbiome composition versus determining a propensity score for an appendix-related condition; etc.), therapies (e.g., different appendix-related characterization models for monitoring efficacy of different therapies, etc.), body sites (e.g., different appendix-related characterization models for processing microorganism datasets corresponding to biological samples from different sample collection sites; etc.), supplementary data, and/or any other suitable components. However, appendix-related characterization models can be tailored and/or used in any suitable manner for facilitating appendix-related characterization and/or therapeutic intervention.

The appendix-related characterization system 220 can preferably determine site-specific appendix-related characterizations (e.g., site-specific analyses). In examples, the appendix-related characterization system 220 can generating and/or apply different site-specific appendix-related characterization models. In specific examples, different site-specific appendix-related characterization models can be generated and/or can be applied based on different microbiome features, such as site-specific features associated with the one or more body sites that the site-specific appendix-related characterization model is associated with (e.g., using gut site-specific features derived from samples collected at gut collection sites of subjects, and correlated with one or more appendix-related conditions, such as for generating a gut site-specific appendix-related characterization model that can be applied for determining characterizations based on user samples collected at user gut collection sites; etc.). Site-specific appendix-related characterization models, site-specific features, samples, site-specific therapies, and/or other suitable entities (e.g., able to be associated with a body site, etc.) are preferably associated with at least one body site (e.g., corresponding to a sample collection site; etc.) including one or more of a gut site (e.g., characterizable based on stool samples, etc.), skin site, nose site, genital site (e.g., associated with genitals, genitalia, etc.), mouth site, and/or any suitable body region. In examples, different appendix-related characterization models can be tailored to different types of inputs, outputs, appendix-related characterizations, appendix-related conditions (e.g., different phenotypic measures that need to be characterized), and/or any other suitable entities. However, site-specific appendix-related characterizations can be configured in any manner and determined in any manner by an appendix-related characterization system 220 and/or other suitable components.

Appendix-related characterization models, other models, other components of embodiments of the system 200, and/or suitable portions of embodiments of the method 100 (e.g., characterization processes, determining microbiome features, determining appendix-related characterizations, etc.) can employ analytical techniques including any one or more of: univariate statistical tests, multivariate statistical tests, dimensionality reduction techniques, artificial intelligence approaches (e.g., machine learning approaches, etc.), performing pattern recognition on data (e.g., identifying correlations between appendix-related conditions and microbiome features; etc.), fusing data from multiple sources (e.g., generating characterization models based on microbiome data and/or supplementary data from a plurality of users associated with one or more appendix-related conditions, such as based on microbiome features extracted from the data; etc.), combination of values (e.g., averaging values, etc.), compression, conversion (e.g., digital-to-analog conversion, analog-to-digital conversion), performing statistical estimation on data (e.g. ordinary least squares regression, non-negative least squares regression, principal components analysis, ridge regression, etc.), wave modulation, normalization, updating (e.g., of characterization models and/or therapy models based on processed biological samples over time; etc.), ranking (e.g., microbiome features; therapies; etc.), weighting (e.g., microbiome features; etc.), validating, filtering (e.g., for baseline correction, data cropping, etc.), noise reduction, smoothing, filling (e.g., gap filling), aligning, model fitting, binning, windowing, clipping, transformations, mathematical operations (e.g., derivatives, moving averages, summing, subtracting, multiplying, dividing, etc.), data association, multiplexing, demultiplexing, interpolating, extrapolating, clustering, image processing techniques, other signal processing operations, other image processing operations, visualizing, and/or any other suitable processing operations. Artificial intelligence approaches can include any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.) reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), an ensemble method (e.g., boosting, boostrapped aggregation, AdaBoost, stacked generalization, gradient boosting machine method, random forest method, etc.), and/or any suitable artificial intelligence approach. However, data processing can be employed in any suitable manner.

The appendix-related characterization system 220 can perform cross-condition analyses for a plurality of appendix-related conditions (e.g., generating multi-condition characterizations based on outputs of different appendix-related characterization models, such as multi-condition microbiome features; etc.). For example, the appendix-related characterization system can characterize relationships between appendix-related conditions based on microorganism data, microbiome features, and/or other suitable microbiome characteristics of users associated with (e.g., diagnosed with, characterized by, etc.) a plurality of appendix-related conditions. In a specific example, cross-condition analyses can be performed based on characterizations for individual appendix-related conditions (e.g., outputs from appendix-related characterization models for individual appendix-related conditions, etc.). Cross-condition analyses can include identification of condition-specific features (e.g., associated exclusively with a single appendix-related condition, etc.), multi-condition features (e.g., associated with two or more appendix-related conditions, etc.), and/or any other suitable types of features. Cross-condition analyses can include determination of parameters informing correlation, concordance, and/or other similar parameters describing relationships between two or more appendix-related conditions, such as by evaluating different pairs of appendix-related conditions. However, the appendix-related characterization system and/or other suitable components can be configured in any suitable manner to facilitate cross-condition analyses (e.g., applying analytical techniques for cross-condition analysis purposes; generating cross-condition characterizations, etc.).

The appendix-related characterization system 220 preferably includes a remote computing system (e.g., for applying appendix-related characterization models, etc.), but can additionally or alternatively include any suitable computing systems (e.g., local computing systems, user devices, handling system components, etc.). However, the appendix-related characterization system 220 can be configured in any suitable manner.

Embodiments of the system 200 can include one or more therapy facilitation systems 230, which can function to facilitate therapeutic intervention (e.g., promote one or more therapies, etc.) for one or more appendix-related conditions (e.g., facilitating modulation of a user microbiome composition and functional diversity for improving a state of the user in relation to one or more appendix-related conditions, etc.). The therapy facilitation system 230 can facilitate therapeutic intervention for any number of appendix-related conditions associated with any number of body sites (e.g., corresponding to any suitable number of collection sites of samples; etc.), such as based on site-specific characterizations (e.g., multi-site characterizations associated with a plurality of body sites; etc.), multi-condition characterizations, other characterizations, and/or any other suitable data. The therapy facilitation system 230 can include any one or more of: a communications system (e.g., to communicate therapy recommendations, selections, discouragements, and/or other suitable therapy-related information to a computing device (e.g., user device and/or care provider device; mobile device; smart phone; desktop computer; at a website, web application, and/or mobile application accessed by the computing device; etc.); to enable telemedicine between a care provider and a subject in relation to an appendix-related condition; etc.), an application executable on a user device (e.g., indicating microbiome composition and/or functionality for a user; etc.), a medical device (e.g., a biological sampling device, such as for collecting samples from different collection sites; medication provision devices; surgical systems; etc.), a user device (e.g., biometric sensors), and/or any other suitable component. One or more therapy facilitation systems 230 can be controllable, communicable with, and/or otherwise associated with the appendix-related characterization system 220. For example, the appendix-related characterization system 220 can generate characterizations of one or more appendix-related conditions for the therapy facilitation system 230 to present (e.g., transmit, communicate, etc.) to a corresponding user (e.g., at an interface 240, etc.). In another example, the therapy facilitation system 230 can update and/or otherwise modify an application and/or other software of a device (e.g., user smartphone) to promote a therapy (e.g., promoting, at a to-do list application, lifestyle changes for improving a user state associated with one or more appendix-related conditions, etc.). However, the therapy facilitation system 230 can be configured in any other manner.

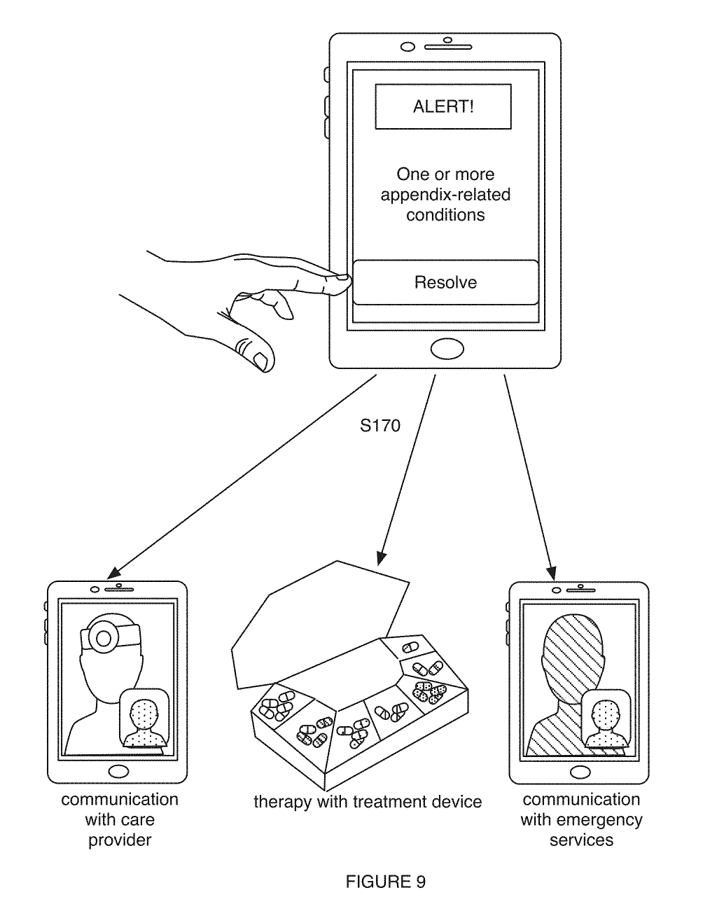

As shown in FIG. 9, embodiments of the system 200 can additionally or alternatively include an interface 240, which can function to improve presentation of microbiome characteristics, appendix-related condition information (e.g., propensity metrics; therapy recommendations; comparisons to other users; other characterizations; etc.), and/or specific information (e.g., any suitable data described herein) associated with (e.g., included in, related to, derivable from, etc.) one or more appendix-related characterizations. In examples, the interface 240 can present appendix-related condition information including a microbiome composition (e.g., taxonomic groups; relative abundances; etc.), functional diversity (e.g., relative abundance of genes associated with particular functions, and propensity metrics for one or more appendix-related conditions, such as relative to user groups sharing a demographic characteristic (e.g., smokers, exercisers, users on different dietary regimens, consumers of probiotics, antibiotic users, groups undergoing particular therapies, etc.). However, the interface 240 can be configured in any suitable manner.

While the components of embodiments of the system 200 are generally described as distinct components, they can be physically and/or logically integrated in any manner. For example, a computing system (e.g., a remote computing system, a user device, etc.) can implement portions and/or all of the appendix-related characterization system 220 (e.g., apply a microbiome-related condition model to generate a characterization of appendix-related conditions for a user, etc.) and the therapy facilitation system 230 (e.g., facilitate therapeutic intervention through presenting insights associated with microbiome composition and/or function; presenting therapy recommendations and/or information; scheduling daily events at a calendar application of the smartphone to notify the user in relation to therapies for improving appendix-related, etc.). In an example, embodiments of the system 200 can omit a therapy facilitation system 230. However, the functionality of embodiments of the system 200 can be distributed in any suitable manner amongst any suitable system components. However, the components of embodiments of the system 200 can be configured in any suitable manner

### 4.1 Determining a Microorganism Dataset.

Embodiments of the method 100 can include Block Silo, which can include determining a microorganism dataset (e.g., microorganism sequence dataset, microbiome composition diversity dataset such as based upon a microorganism sequence dataset, microbiome functional diversity dataset such as based upon a microorganism sequence dataset, etc.) associated with a set of users Silo. Block Silo can function to process samples (e.g., biological samples; non-biological samples; an aggregate set of samples associated with a population of subjects, a subpopulation of subjects, a subgroup of subjects sharing a demographic characteristic and/or other suitable characteristics; a user sample; etc.), in order to determine compositional, functional, pharmacogenomics, and/or other suitable aspects associated with the corresponding microbiomes, such as in relation to one or more appendix-related conditions. Compositional and/or functional aspects can include one or more of aspects at the microorganism level (and/or other suitable granularity), including parameters related to distribution of microorganisms across different groups of kingdoms, phyla, classes, orders, families, genera, species, subspecies, strains, and/or any other suitable infraspecies taxon (e.g., as measured in total abundance of each group, relative abundance of each group, total number of groups represented, etc.). Compositional and/or functional aspects can also be represented in terms of operational taxonomic units (OTUs). Compositional and/or functional aspects can additionally or alternatively include compositional aspects at the genetic level (e.g., regions determined by multilocus sequence typing, 16S sequences, 18S sequences, ITS sequences, other genetic markers, other phylogenetic markers, etc.). Compositional and functional aspects can include the presence or absence or the quantity of genes associated with specific functions (e.g. enzyme activities, transport functions, immune activities, etc.). Outputs of Block Silo can thus be used to facilitate determination of microbiome features (e.g., generation of a microorganism sequence dataset usable for identifying microbiome features; etc.) for the characterization process of Block S130 and/or other suitable portions of embodiments of the method 100 (e.g., where Block Silo can lead to outputs of microbiome composition datasets, microbiome functional datasets, and/or other suitable microorganism datasets from which microbiome features can be extracted, etc.), where the features can be microorganism-based (e.g., presence of a genus of bacteria), genetic-based (e.g., based upon representation of specific genetic regions and/or sequences), functional-based (e.g., presence of a specific catalytic activity), and/or any other suitable microbiome features.

In a variation, Block Silo can include assessment and/or processing based upon phylogenetic markers (e.g., for generating microorganism datasets, etc.) derived from bacteria and/or archaea in relation to gene families associated with one or more of: ribosomal protein S2, ribosomal protein S3, ribosomal protein S5, ribosomal protein S7, ribosomal protein S8, ribosomal protein S9, ribosomal protein S10, ribosomal protein S11, ribosomal protein S12/S23, ribosomal protein S13, ribosomal protein S15P/S13e, ribosomal protein S17, ribosomal protein S19, ribosomal protein L1, ribosomal protein L2, ribosomal protein L3, ribosomal protein L4/Lie, ribosomal protein L5, ribosomal protein L6, ribosomal protein L10, ribosomal protein L11, ribosomal protein L14b/L23e, ribosomal protein L15, ribosomal protein L16/L10E, ribosomal protein L18P/L5E, ribosomal protein L22, ribosomal protein L24, ribosomal protein L25/L23, ribosomal protein L29, translation elongation factor EF-2, translation initiation factor IF-2, metalloendopeptidase, ffh signal recognition particle protein, phenylalanyl-tRNA synthetase beta subunit, phenylalanyl-tRNA synthetase alpha subunit, tRNA pseudouridine synthase B, Porphobilinogen deaminase, ribosomal protein L13, phosphoribosylformylglycinamidine cyclo-ligase, and ribonuclease HII. Additionally or alternatively, markers can include target sequences (e.g., sequences associated with a microorganism taxonomic group; sequences associated with functional aspects; sequences correlated with appendix-related conditions; sequences indicative of user responsiveness to different therapies; sequences that are invariant across a population and/or any suitable set of subjects, such as to facilitate multiplex amplification using a primer type sharing a primer sequence; conserved sequences; sequences including mutations, polymorphisms; nucleotide sequences; amino acid sequences; etc.), proteins (e.g., serum proteins, antibodies, etc.), peptides, carbohydrates, lipids, other nucleic acids, whole cells, metabolites, natural products, genetic predisposition biomarkers, diagnostic biomarkers, prognostic biomarkers, predictive biomarkers, other molecular biomarkers, gene expression markers, imaging biomarkers, and/or other suitable markers. However, markers can include any other suitable marker(s) associated with microbiome composition, microbiome functionality, and/or appendix-related conditions.

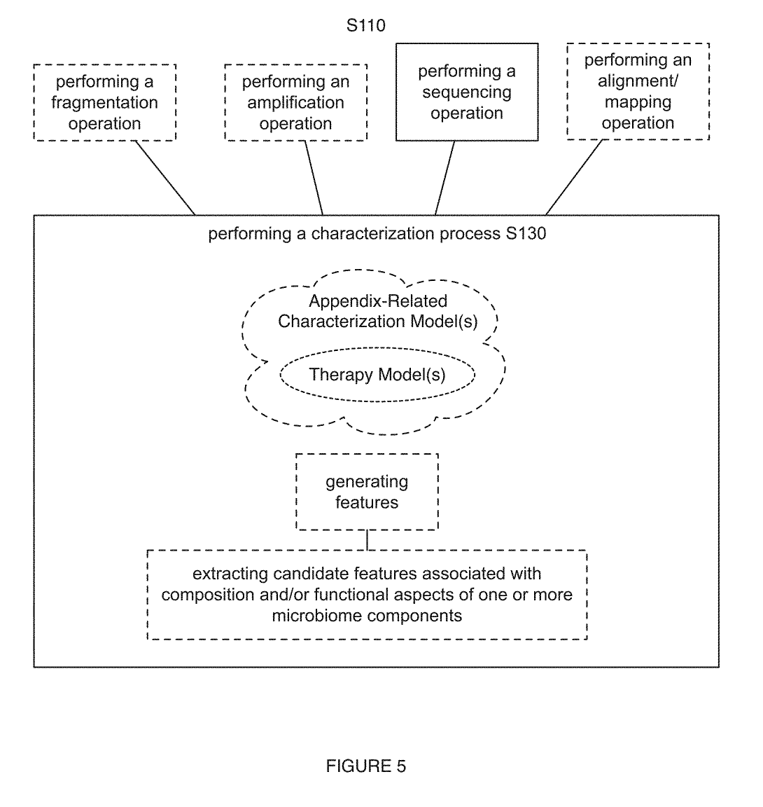

Characterizing the microbiome composition and/or functional aspects for each of the aggregate set of biological samples thus preferably includes a combination of sample processing techniques (e.g., wet laboratory techniques; as shown in FIG. 5), including, but not limited to, amplicon sequencing (e.g., 16S, 18S, ITS), UMIs, 3 step PCR, CRISPR, metagenomic approaches, metatranscriptomics, use of random primers, and computational techniques (e.g., utilizing tools of bioinformatics), to quantitatively and/or qualitatively characterize the microbiome and functional aspects associated with each biological sample from a subject or population of subjects. For example, determining a microorganism dataset (e.g., microorganism sequence dataset, etc.) can include determining at least one of a metagenomic library and a metatranscriptomic library based on microorganism nucleic acids of one or more samples (e.g., at least a subset of the microorganism nucleic acids present in the sample; etc.), and where determining a set of microbiome features can be based on the at least one of the metagenomic library and the metatranscriptomic library.

In variations, sample processing in Block Silo can include any one or more of: lysing a biological sample, disrupting membranes in cells of a biological sample, separation of undesired elements (e.g., RNA, proteins) from the biological sample, purification of nucleic acids (e.g., DNA) in a biological sample, amplification of nucleic acids from the biological sample, further purification of amplified nucleic acids of the biological sample, and sequencing of amplified nucleic acids of the biological sample. In an example, Block Silo can include: collecting biological samples from a set of users (e.g., biological samples collected by the user with a sampling kit including a sample container, etc.), where the biological samples include microorganism nucleic acids associated with the appendix-related condition (e.g., microorganism nucleic acids including target sequences correlated with an appendix-related condition; etc.). In another example, Block Silo can include providing a set of sampling kits to a set of users, each sampling kit of the set of sampling kits including a sample container (e.g., including pre-processing reagents, such as lysing reagents; etc.) operable to receive a biological sample from a user of the set of users.

In variations, lysing a biological sample and/or disrupting membranes in cells of a biological sample preferably includes physical methods (e.g., bead beating, nitrogen decompression, homogenization, sonication), which omit certain reagents that produce bias in representation of certain bacterial groups upon sequencing. Additionally or alternatively, lysing or disrupting in Block Silo can involve chemical methods (e.g., using a detergent, using a solvent, using a surfactant, etc.). Additionally or alternatively, lysing or disrupting in Block Silo can involve biological methods. In variations, separation of undesired elements can include removal of RNA using RNases and/or removal of proteins using proteases. In variations, purification of nucleic acids can include one or more of: precipitation of nucleic acids from the biological samples (e.g., using alcohol-based precipitation methods), liquid-liquid based purification techniques (e.g., phenol-chloroform extraction), chromatography-based purification techniques (e.g., column adsorption), purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.), and any other suitable purification techniques.

In variations, amplification of purified nucleic acids can include one or more of: polymerase chain reaction (PCR)-based techniques (e.g., solid-phase PCR, RT-PCR, qPCR, multiplex PCR, touchdown PCR, nanoPCR, nested PCR, hot start PCR, etc.), helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and any other suitable amplification technique. In amplification of purified nucleic acids, the primers used are preferably selected to prevent or minimize amplification bias, as well as configured to amplify nucleic acid regions/sequences (e.g., of the 16S region, the 18S region, the ITS region, etc.) that are informative taxonomically, phylogenetically, for diagnostics, for formulations (e.g., for probiotic formulations), and/or for any other suitable purpose. Thus, universal primers (e.g., a F27-R338 primer set for 16S RNA, a F515-R8006 primer set for 16S RNA, etc.) configured to avoid amplification bias can be used in amplification. Additionally or alternatively include incorporated barcode sequences and/or UMIs specific to biological samples, to users, to appendix-related conditions, to taxa, to target sequences, and/or to any other suitable components, which can facilitate a post-sequencing identification process (e.g., for mapping sequence reads to microbiome composition and/or microbiome function aspects; etc.). Primers used in variations of Block Silo can additionally or alternatively include adaptor regions configured to cooperate with sequencing techniques involving complementary adaptors (e.g., Illumina Sequencing). Additionally or alternatively, Block Silo can implement any other step configured to facilitate processing (e.g., using a Nextera kit). In a specific example, performing amplification and/or sample processing operations can be in a multiplex manner (e.g., for a single biological sample, for a plurality of biological samples across multiple users; etc.). In another specific example, performing amplification can include normalization steps to balance libraries and detect all amplicons in a mixture independent of the amount of starting material, such as 3 step PCR, bead based normalization, and/or other suitable techniques.

In variations, sequencing of purified nucleic acids can include methods involving targeted amplicon sequencing, metatranscriptomic sequencing, and/or metagenomic sequencing, implementing techniques including one or more of: sequencing-by-synthesis techniques (e.g., Illumina sequencing), capillary sequencing techniques (e.g., Sanger sequencing), pyrosequencing techniques, and nanopore sequencing techniques (e.g., using an Oxford Nanopore technique).

In a specific example, amplification and sequencing of nucleic acids from biological samples of the set of biological samples includes: solid-phase PCR involving bridge amplification of DNA fragments of the biological samples on a substrate with oligo adapters, where amplification involves primers having a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, UMIs, a sequence for targeting a specific target region (e.g., 16S region, 18S region, ITS region), a reverse index sequence (e.g., corresponding to an Illumina reverse index for MiSeq/HiSeq platforms), and a reverse barcode sequence. In the specific example, sequencing can include Illumina sequencing (e.g., with a HiSeq platform, with a MiSeq platform, with a NextSeq platform, etc.) using a sequencing-by-synthesis technique. In another specific example, the method 100 can include: identifying one or more primer types compatible with one or more genetic targets associated with one or more appendix-related conditions (e.g., a biomarker of the one or more appendix-related conditions; positively correlated with; negatively correlated with; causative of; etc.); determining a microorganism dataset (e.g., microorganism sequence dataset; such as with a next-generation sequencing system; etc.) for one or more users (e.g., set of subjects) based on the one or more primer types (e.g., based on primers corresponding to the one or more primer types, and on the microorganism nucleic acids included in collected biological samples, etc.), such as through fragmenting the microorganism nucleic acids, and/or performing a singleplex amplification process and/or a multiplex amplification process for the fragmented microorganism nucleic acids based on the one or more identified primer types (e.g., primers corresponding to the primer types, etc.) compatible with the one or more genetic targets associated with the appendix-related condition; and/or promoting (e.g., providing), based on an appendix-related characterization derived from a microorganism dataset, a therapy for the user condition (e.g., for the appendix-related condition; enabling selective modulation of a microbiome of the user in relation to at least one of a population size of a desired taxon and a desired microbiome function, etc.). In a specific example, where determining the microorganism dataset can include generating amplified microorganism nucleic acids through at least one of a singleplex amplification process and a multiplex amplification process for the microorganism nucleic acids; and determining, with a next-generation sequencing system, the microorganism dataset based on the amplified microorganism nucleic acids.

In examples, the biological samples can correspond to a one or more collection sites including at least one of a gut collection site (e.g., corresponding to a body site type of a gut site), a skin collection site (e.g., corresponding to a body site type of a skin site), a nose collection site (e.g., corresponding to a body site type of a nose site), a mouth collection site (e.g., corresponding to a body site type of a mouth site), and a genitals collection site (e.g., corresponding to a body site type of a genital site). In a specific example, determining a microorganism dataset (e.g., microorganism sequence dataset, etc.) can include identifying a first primer type compatible with a first genetic target associated with one or more appendix-related conditions and a first collection site of the set of collection sites; identifying a second primer type compatible with a second genetic target associated with the one or more appendix-related conditions and a second collection site of the set of collection sites; and generating the microorganism dataset for the set of subjects based on the microorganism nucleic acids, the first primers corresponding to the first primer type, and second primers corresponding to the second primer type.

In variations, primers (e.g., of a primer type corresponding to a primer sequence; etc.) used in Block Silo and/or other suitable portions of embodiments of the method 100 can include primers associated with protein genes (e.g., coding for conserved protein gene sequences across a plurality of taxa, such as to enable multiplex amplification for a plurality of targets and/or taxa; etc.). Primers can additionally or alternatively be associated with appendix-related conditions (e.g., primers compatible with genetic targets including microorganism sequence biomarkers for microorganisms correlated with appendix-related conditions; etc.), microbiome composition features (e.g., identified primers compatible with a genetic target corresponding to microbiome composition features associated with a group of taxa correlated with an appendix-related condition; genetic sequences from which relative abundance features are derived etc.), functional diversity features, supplementary features, and/or other suitable features and/or data. Primers (and/or other suitable molecules, markers, and/or biological material described herein) can possess any suitable size (e.g., sequence length, number of base pairs, conserved sequence length, variable region length, etc.). Additionally or alternatively, any suitable number of primers can be used in sample processing for performing characterizations (e.g., appendix-related characterizations; etc.), improving sample processing (e.g., through reducing amplification bias, etc.), and/or for any suitable purposes. The primers can be associated with any suitable number of targets, sequences, taxa, conditions, and/or other suitable aspects. Primers used in Block S100 and/or other suitable portions of embodiments of the method 100 can be selected through processes described in Block Silo (e.g., primer selection based on parameters used in generating the taxonomic database) and/or any other suitable portions of embodiments of the method 100. Additionally or alternatively, primers (and/or processes associated with primers) can include and/or be analogous to that described in U.S. application Ser. No. 14/919,614, filed 21 Oct. 2015, which is herein incorporated in its entirety by this reference. However, identification and/or usage of primers can be configured in any suitable manner.

Some variations of sample processing can include further purification of amplified nucleic acids (e.g., PCR products) prior to sequencing, which functions to remove excess amplification elements (e.g., primers, dNTPs, enzymes, salts, etc.). In examples, additional purification can be facilitated using any one or more of: purification kits, buffers, alcohols, pH indicators, chaotropic salts, nucleic acid binding filters, centrifugation, and/or any other suitable purification technique.

In variations, computational processing in Block Silo can include any one or more of: identification of microbiome-derived sequences (e.g., as opposed to subject sequences and contaminants), alignment and mapping of microbiome-derived sequences (e.g., alignment of fragmented sequences using one or more of single-ended alignment, ungapped alignment, gapped alignment, pairing), and generating features associated with (e.g., derived from) compositional and/or functional aspects of the microbiome associated with a biological sample.

Identification of microbiome-derived sequences can include mapping of sequence data from sample processing to a subject reference genome (e.g., provided by the Genome Reference Consortium), in order to remove subject genome-derived sequences. Unidentified sequences remaining after mapping of sequence data to the subject reference genome can then be further clustered into operational taxonomic units (OTUs) based upon sequence similarity and/or reference-based approaches (e.g., using VAMPS, using MG-RAST, using QIIME databases), aligned (e.g., using a genome hashing approach, using a Needleman-Wunsch algorithm, using a Smith-Waterman algorithm), and mapped to reference bacterial genomes (e.g., provided by the National Center for Biotechnology Information), using an alignment algorithm (e.g., Basic Local Alignment Search Tool, FPGA accelerated alignment tool, BWT-indexing with BWA, BWT-indexing with SOAP, BWT-indexing with Bowtie, etc.). Mapping of unidentified sequences can additionally or alternatively include mapping to reference archaeal genomes, viral genomes and/or eukaryotic genomes. Furthermore, mapping of taxa can be performed in relation to existing databases, and/or in relation to custom-generated databases.

However, processing biological samples, generating a microorganism dataset, and/or other aspects associated with Block Silo can be performed in any suitable manner.

### 4.2 Processing Supplementary Data.

Embodiments of the method 100 can additionally or alternatively include Block S120, which can include processing (e.g., receiving, collecting, transforming, determining supplementary features, ranking supplementary features, identifying correlations, etc.) supplementary data (e.g., one or more supplementary datasets, etc.) associated with (e.g., informative of; describing; indicative of; correlated with; etc.) one or more appendix-related conditions, one or more users, and/or other suitable entities. Block S120 can function to process data for supplementing microorganism datasets, microbiome features (e.g., in relation to determining appendix-related characterizations and/or facilitating therapeutic intervention, etc.), and/or can function to supplement any suitable portion of the method 100 and/or system 200 (e.g., processing supplementary data for facilitating one or more characterization processes, such as in Block S130; such as for facilitating training, validating, generating, determining, applying and/or otherwise processing appendix-related characterization models, etc.). In an example, supplementary data can include at least one of survey-derived data, user data, site-specific data, and device data (and/or other suitable supplementary data), where an example of method 100 can include determining a set of supplementary features based on the at least one of the survey-derived data, the user data, the site-specific data, and the device data (and/or other suitable supplementary data); and generating one or more appendix-related characterization models based on the supplementary features, microbiome features, and/or other suitable data.

Supplementary data can include any one or more of: survey-derived data (e.g., data from responses to one or more surveys surveying for one or more appendix-related conditions, for any suitable types of data described herein; etc.); site-specific data (e.g., data informative of different collection sites, such as prior biological knowledge indicating correlations between microbiomes at specific collection sites and one or more appendix-related conditions; etc.); appendix-related condition data (e.g., data informative of different appendix-related conditions, such as in relation to microbiome characteristics, therapies, users, etc.); device data (e.g., sensor data; contextual sensor data associated with appendix; wearable device data; medical device data; user device data such as mobile phone application data; web application data; etc.); user data (e.g., user medical data current and historical medical data such as historical therapies, historical medical examination data; medical device-derived data; physiological data; data associated with medical tests; social media data; demographic data; family history data; behavior data describing behaviors; environmental factor data describing environmental factors; diet-related data such as data from food establishment check-ins, data from spectrophotometric analysis, user-inputted data, nutrition data associated with probiotic and/or prebiotic food items, types of food consumed, amount of food consumed, caloric data, diet regimen data, and/or other suitable diet-related data; etc.); prior biological knowledge (e.g., informative of appendix-related conditions, microbiome characteristics, associations between microbiome characteristics and appendix-related conditions, etc.); and/or any other suitable type of supplementary data.

In variations, processing supplementary data can include processing survey-derived data, where the survey-derived data can provide physiological data, demographic data, behavior data, environmental factor data (e.g., describing environmental factors, etc.), other types of supplementary data, and/or any other suitable data. Physiological data can include information related to physiological features (e.g., height, weight, body mass index, body fat percent, body hair level, medical history, etc.). Demographic data can include information related to demographic characteristics (e.g., gender, age, ethnicity, marital status, number of siblings, socioeconomic status, sexual orientation, etc.). Behavioral data can describe behaviors including one or more: health-associated states (e.g., health and disease states), dietary habits (e.g., alcohol consumption, caffeine consumption, omnivorous, vegetarian, vegan, sugar consumption, acid consumption, consumption of wheat, egg, soy, treenut, peanut, shellfish, food preferences, allergy characteristics, consumption and/or avoidance of other food items, etc.), behavioral tendencies (e.g., levels of physical activity, drug use, alcohol use, habit development, etc.), different levels of mobility (e.g., amount of exercise such as low, moderate, and/or extreme physical exercise activity; related to distance traveled within a given time period; indicated by mobility sensors such as motion and/or location sensors; etc.), different levels of sexual activity (e.g., related to numbers of partners and sexual orientation), and any other suitable behavioral data. Survey-derived data can include quantitative data, qualitative data, and/or other suitable types of survey-derived data, such as where qualitative data can be converted to quantitative data (e.g., using scales of severity, mapping of qualitative responses to quantified scores, etc.). Processing survey-derived data can include facilitating collection of survey-derived data, such as by providing one or more surveys to one or more users, subjects, and/or other suitable entities. Surveys can be provided in-person (e.g., in coordination with sample kit provision and/or reception of samples; etc.), electronically (e.g., during account setup; at an application executing at an electronic device of a subject, at a web application and/or website accessible through an internet connection; etc.), and/or in any other suitable manner.

Additionally or alternatively, processing supplementary data can include processing sensor data (e.g., sensors of appendix-related devices, wearable computing devices, mobile devices; biometric sensors associated with the user, such as biometric sensors of a user smart phone; etc.). Sensor data can include any one or more of: physical activity- and/or physical action-related data (e.g., accelerometer data, gyroscope data, location sensor data such as GPS data, and/or other mobility sensor data from one or more devices such as a mobile device and/or wearable electronic device, etc.), sensor data describing environmental factors (e.g., temperature data, elevation data, climate data, light parameter data, pressure data, air quality data, etc.), biometric sensor data (e.g., blood pressure data; temperature data; pressure data associated with swelling; heart rate sensor data; fingerprint sensor data; optical sensor data such as facial images and/or video; data recorded through sensors of a mobile device; data recorded through a wearable or other peripheral device; etc.), and/or any other suitable data associated with sensors. Additionally or alternatively, sensor data can include data sampled at one or more: optical sensors (e.g., image sensors, light sensors, cameras, etc.), audio sensors (e.g., microphones, etc.), temperature sensors, volatile compound sensors, air quality sensors, weight sensors, humidity sensors, depth sensors, location sensors (GPS receivers; beacons; indoor positioning systems; compasses; etc.), motion sensors (e.g., accelerators, gyroscope, magnetometer, motion sensors integrated with a device worn by a user, etc.), biometric sensors (e.g., heart rate sensors such as for monitoring heart rate; fingerprint sensors; facial recognition sensors; bio-impedance sensors, etc.), pressure sensors, proximity sensors (e.g., for monitoring motion and/or other aspects of third-party objects associated with user appendix periods; etc.), flow sensors, power sensors (e.g., Hall effect sensors), virtual reality-related sensors, augmented reality-related sensors, and/or or any other suitable types of sensors.

Additionally or alternatively, supplementary data can include medical record data and/or clinical data. As such, portions of the supplementary dataset can be derived from one or more electronic health records (EHRs). Additionally or alternatively, supplementary data can include any other suitable diagnostic information (e.g., clinical diagnosis information). Any suitable supplementary data (e.g., in the form of extracted supplementary features, etc.) can be combined with and/or used with microbiome features and/or other suitable data for performing portions of embodiments of the method 100 (e.g., performing characterization processes, etc.) and/or system 200. For example, supplementary data associated with (e.g., derived from, etc.) computed tomography (CT scan), ultrasound, colonoscopy, biopsy, blood test, abdominal exam (e.g., to detect inflammation, etc.), urine test (e.g., to detect infection; etc.), diagnostic imaging, other suitable diagnostic procedures associated with appendix-related conditions, survey-related information, and/or any other suitable test can be used to supplement (e.g., for any suitable portions of embodiments of the method 100 and/or system 200).

Additionally or alternatively, supplementary data can include therapy-related data including one or more of: therapy regimens, types of therapies, recommended therapies, therapies used by the user, therapy adherence, and/or other suitable data related to therapies. For example, supplementary data can include user adherence metrics (e.g., medication adherence, probiotic adherence, physical exercise adherence, dietary adherence, etc.) in relation one or more therapies (e.g., a recommended therapy, etc.). However, processing supplementary data can be performed in any suitable manner.

### 4.3 Performing a Characterization Process.

Embodiments of the method 100 can include Block S130, which can include, performing a characterization process (e.g., pre-processing; feature generation; feature processing; site-specific characterization, such as characterization specific to one or more particular body sites, such as for samples collected at collection sites corresponding to the body site, such as multi-site characterization for a plurality of body sites; cross-condition analysis for a plurality of appendix-related conditions; model generation; etc.) associated with one or more appendix-related conditions, such as based on a microorganism dataset (e.g., derived in Block Silo, etc.) and/or other suitable data (e.g., supplementary dataset; etc.) S130. Block S130 can function to identify, determine, extract, and/or otherwise process features and/or feature combinations that can be used to determine appendix-related characterizations for users or and sets of users, based upon their microbiome composition (e.g., microbiome composition diversity features, etc.), function (e.g., microbiome functional diversity features, etc.), and/or other suitable microbiome features (e.g., such as through the generation and application of a characterization model for determining appendix-related characterizations, etc.). As such, the characterization process can be used as a diagnostic tool that can characterize a subject (e.g., in terms of behavioral traits, in terms of medical conditions, in terms of demographic characteristics, etc.) based upon their microbiome composition and/or functional features, in relation to one or more of their health condition states (e.g., appendix-related condition states), behavioral traits, medical conditions, demographic characteristics, and/or any other suitable traits. Such characterizations can be used to determine, recommend, and/or provide therapies (e.g., personalized therapies, such as determined by way of a therapy model, etc.), and/or otherwise facilitate therapeutic intervention.

Performing a characterization process S130 can include pre-processing microorganism datasets, microbiome features, and/or other suitable data for facilitation of downstream processing (e.g., determining appendix-related characterizations, etc.). In an example, performing a characterization process can include, filtering a microorganism dataset (e.g., filtering a microorganism sequence dataset, such as prior to applying a set of analytical techniques to determine the microbiome features, etc.), by at least one of: a) removing first sample data corresponding to first sample outliers of a set of biological samples (e.g., associated with one or more appendix-related conditions, etc.), such as where the first sample outliers are determined by at least one of principal component analysis, a dimensionality reduction technique, and a multivariate methodology; b) removing second sample data corresponding to second sample outliers of the set of biological samples, where the second sample outliers can determined based on corresponding data quality for the set of microbiome features (e.g., removing samples corresponding to a number of microbiome features with high quality data below a threshold condition, etc.); and c) removing one or more microbiome features from the set of microbiome features based on a sample number for the microbiome feature failing to satisfy a threshold sample number condition, where the sample number corresponds to a number of samples associated with high quality data for the microbiome feature. However, pre-processing can be performed with any suitable analytical techniques in any suitable manner.

In performing the characterization process, Block S130 can use computational methods (e.g., statistical methods, machine learning methods, artificial intelligence methods, bioinformatics methods, etc.) to characterize a subject as exhibiting features (e.g., where determining user microbiome features can include determining feature values for microbiome features identified by characterization processes as correlated with and/or otherwise associated with one or more appendix-related conditions, etc.) associated with one or more appendix-related conditions (e.g., features characteristic of a set of users with the one or more appendix-related conditions, etc.).

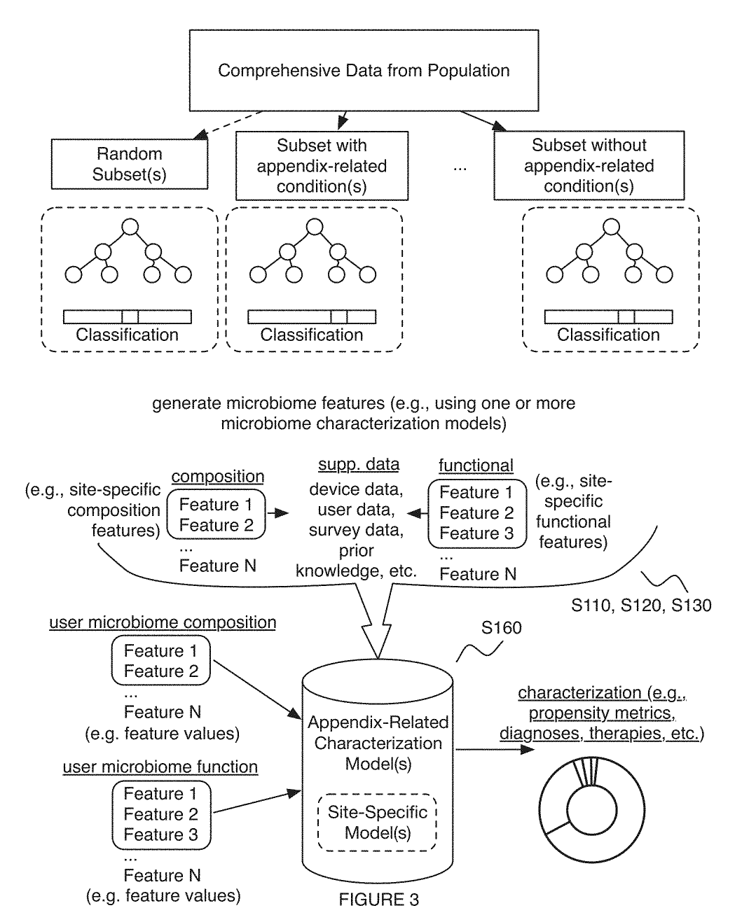

As shown in FIG. 3, performing characterization processes can include determining one or more microbiome features associated with one or more appendix-related conditions (e.g., identifying microbiome features with greatest relevance to one or more appendix-related conditions; determining user microbiome features, such as presence, absence, and/or values of user microbiome features corresponding to the identified microbiome features associated with the one or more appendix-related conditions, etc.), such as through applying one or more analytical techniques. In an example, determining microbiome features (e.g., microbiome composition features, microbiome functional features, etc.) can applying a set of analytical techniques including at least one of a univariate statistical test, a multivariate statistical test, a dimensionality reduction technique, and an artificial intelligence approach, such as based on a microorganism dataset (e.g., microorganism sequence dataset, etc.), and where the microbiome features can be configured to improve computing system-related functionality associated with the determining of the appendix-related characterization for the user (e.g., in relation to accuracy, reducing error, processing speed, scaling, etc.). In an example, determining microbiome features (e.g., user microbiome features, etc.) can include applying a set of analytical techniques to determine at least one of presence of at least one of a microbiome composition diversity feature and a microbiome functional diversity feature, absence of the at least one of the microbiome composition diversity feature and the microbiome functional diversity feature, a relative abundance feature describing relative abundance of different taxonomic groups associated with the first appendix-related condition, a ratio feature describing a ratio between at least two microbiome features associated with the different taxonomic groups, an interaction feature describing an interaction between the different taxonomic groups, and a phylogenetic distance feature describing phylogenetic distance between the different taxonomic groups, such as based on the microorganism dataset, and where the set of analytical techniques can include at least one of a univariate statistical test, a multivariate statistical test, a dimensionality reduction technique, and an artificial intelligence approach.

In variations, upon identification of represented groups of microorganisms of the microbiome associated with a biological sample, generating features associated with (e.g., derived from) compositional and functional aspects of the microbiome associated with a biological sample can be performed. In a variation, generating features can include generating features based upon multilocus sequence typing (MSLT), in order to identify markers useful for characterization in subsequent blocks of the method 100. Additionally or alternatively, generated features can include generating features that describe the presence or absence of certain taxonomic groups of microorganisms, and/or ratios between exhibited taxonomic groups of microorganisms. Additionally or alternatively, generating features can include generating features describing one or more of: quantities of represented taxonomic groups, networks of represented taxonomic groups, correlations in representation of different taxonomic groups, interactions between different taxonomic groups, products produced by different taxonomic groups, interactions between products produced by different taxonomic groups, ratios between dead and alive microorganisms (e.g., for different represented taxonomic groups, based upon analysis of RNAs), phylogenetic distance (e.g., in terms of Kantorovich-Rubinstein distances, Wasserstein distances etc.), any other suitable taxonomic group-related feature(s), any other suitable genetic or functional aspect(s).

Additionally or alternatively, generating features can include generating features describing relative abundance of different microorganism groups, for instance, using a sparCC approach, using Genome Relative Abundance and Average size (GAAS) approach and/or using a Genome Relative Abundance using Mixture Model theory (GRAMMy) approach that uses sequence-similarity data to perform a maximum likelihood estimation of the relative abundance of one or more groups of microorganisms. Additionally or alternatively, generating features can include generating statistical measures of taxonomic variation, as derived from abundance metrics. Additionally or alternatively, generating features can include generating features associated with (e.g., derived from) relative abundance factors (e.g., in relation to changes in abundance of a taxon, which affects abundance of other taxa). Additionally or alternatively, generating features can include generation of qualitative features describing presence of one or more taxonomic groups, in isolation and/or in combination. Additionally or alternatively, generating features can include generation of features related to genetic markers (e.g., representative 16S, 18S, and/or ITS sequences) characterizing microorganisms of the microbiome associated with a biological sample. Additionally or alternatively, generating features can include generation of features related to functional associations of specific genes and/or organisms having the specific genes. Additionally or alternatively, generating features can include generation of features related to pathogenicity of a taxon and/or products attributed to a taxon. Block S130 can, however, include determination of any other suitable feature(s) derived from sequencing and mapping of nucleic acids of a biological sample. For instance, the feature(s) can be combinatory (e.g. involving pairs, triplets), correlative (e.g., related to correlations between different features), and/or related to changes in features (e.g., temporal changes, changes across sample sites, etc., spatial changes, etc.). However, determining microbiome features can be performed in any suitable manner.

In variations, performing a characterization process can include performing one or more multi-site analyses (e.g., with appendix-related characterization models; generating a multi-site characterization, etc.) associated with a plurality of collection sites, such as performing appendix-related characterizations based on a set of site-specific features including a first subset of site-specific features associated with a first body site, and a second subset of site-specific features associated with a second body site. However, multi-site analyses can be performed in any suitable manner.

In variations, performing a characterization process can include performing one or more cross-condition analyses (e.g., using appendix-related characterization models, etc.) for a plurality of appendix-related conditions. In an example, performing cross-condition analyses can include determining a set of cross-condition features (e.g., as part of determining microbiome features, etc.) associated with a plurality of appendix-related conditions (e.g., a first appendix-related condition and a second appendix-related condition, etc.) based on one or more analytical techniques, where determining an appendix-related characterization can include determining the appendix-related characterization for a user for the plurality of appendix-related conditions (e.g., first and the second appendix-related conditions, etc.) based on one or more appendix-related characterization models, and where the set of cross-condition features is configured to improve the computing system-related functionality associated with the determining of the appendix-related characterization for the user for the plurality of appendix-related conditions. Performing cross-condition analyses can include determining cross-condition correlation metrics (e.g., correlation and/or covariance between data corresponding to different appendix-related conditions, etc.) and/or other suitable metrics associated with cross-condition analyses. However, performing cross-condition analyses can be performed in any suitable manner.

In a variation, characterization can be based upon features associated with (e.g., derived from) a statistical analysis (e.g., an analysis of probability distributions) of similarities and/or differences between a first group of subjects exhibiting a target state (e.g., an appendix-related condition state) and a second group of subjects not exhibiting the target state (e.g., a “normal” state). In implementing this variation, one or more of a Kolmogorov-Smirnov (KS) test, a permutation test, a Cramer-von Mises test, any other statistical test (e.g., t-test, z-test, chi-squared test, test associated with distributions, etc.), and/or other suitable analytical techniques can be used. In particular, one or more such statistical hypothesis tests can be used to assess a set of features having varying degrees of abundance in a first group of subjects exhibiting a target state (e.g., a sick state) and a second group of subjects not exhibiting the target state (e.g., having a normal state). In more detail, the set of features assessed can be constrained based upon percent abundance and/or any other suitable parameter pertaining to diversity in association with the first group of subjects and the second group of subjects, in order to increase or decrease confidence in the characterization. In a specific implementation of this example, a feature can be derived from a taxon of bacteria that is abundant in a certain percentage of subjects of the first group and subjects of the second group, where a relative abundance of the taxon between the first group of subjects and the second group of subjects can be determined from the KS test, with an indication of significance (e.g., in terms of p-value). Thus, an output of Block S130 can include a normalized relative abundance value (e.g., 25% greater abundance of a taxon in subjects with an appendix-related condition vs. subjects without the appendix-related condition; in sick subjects vs. healthy subjects) with an indication of significance (e.g., a p-value of 0.0013). Variations of feature generation can additionally or alternatively implement or be derived from functional features or metadata features (e.g., non-bacterial markers). Additionally or alternatively, any suitable microbiome features can be derived based on statistical analyses (e.g., applied to a microorganism sequence dataset and/or other suitable microorganism dataset, etc.) including any one or more of: a prediction analysis, multi hypothesis testing, a random forest test, principal component analysis, and/or other suitable analytical techniques.

In performing the characterization process, Block S130 can additionally or alternatively transform input data from at least one of the microbiome composition diversity dataset and microbiome functional diversity dataset into feature vectors that can be tested for efficacy in predicting characterizations of the population of subjects. Data from the supplementary dataset can be used to provide indication of one or more characterizations of a set of characterizations, where the characterization process is trained with a training dataset of candidate features and candidate classifications to identify features and/or feature combinations that have high degrees (or low degrees) of predictive power in accurately predicting a classification. As such, refinement of the characterization process with the training dataset identifies feature sets (e.g., of subject features, of combinations of features) having high correlation with specific classifications of subjects.

In variations, feature vectors (and/or any suitable set of features) effective in predicting classifications of the characterization process can include features related to one or more of: microbiome diversity metrics (e.g., in relation to distribution across taxonomic groups, in relation to distribution across archaeal, bacterial, viral, and/or eukaryotic groups), presence of taxonomic groups in one's microbiome, representation of specific genetic sequences (e.g., 16S sequences) in one's microbiome, relative abundance of taxonomic groups in one's microbiome, microbiome resilience metrics (e.g., in response to a perturbation determined from the supplementary dataset), abundance of genes that encode proteins or RNAs with given functions (enzymes, transporters, proteins from the immune system, hormones, interference RNAs, etc.) and any other suitable features associated with (e.g., derived from) the microbiome diversity dataset and/or the supplementary dataset. In variations, microbiome features can be associated with (e.g., include, correspond to, typify, etc.) at least one of: presence of a microbiome feature from the microbiome features (e.g., user microbiome features, etc.), absence of the microbiome features from the microbiome features, relative abundance of different taxonomic groups associated with the appendix-related condition; a ratio between at least two microbiome features associated with the different taxonomic groups, interactions between the different taxonomic groups, and phylogenetic distance between the different taxonomic groups. In a specific example, microbiome features can include one or more relative abundance characteristics associated with at least one of the microbiome composition diversity features (e.g., relative abundance associated with different taxa, etc.) and the microbiome functional diversity features (e.g., relative abundance of sequences corresponding to different functional features; etc.). Relative abundance characteristics and/or other suitable microbiome features (and/or other suitable data described herein) can be extracted and/or otherwise determined based on: a normalization, a feature vector derived from at least one of linear latent variable analysis and non-linear latent variable analysis, linear regression, non-linear regression, a kernel method, a feature embedding method, a machine learning method, a statistical inference method, and/or other suitable analytical techniques. Additionally or alternatively, combinations of features can be used in a feature vector, where features can be grouped and/or weighted in providing a combined feature as part of a feature set. For example, one feature or feature set can include a weighted composite of the number of represented classes of bacteria in one's microbiome, presence of a specific genus of bacteria in one's microbiome, representation of a specific 16S sequence in one's microbiome, and relative abundance of a first phylum over a second phylum of bacteria. However, the feature vectors can additionally or alternatively be determined in any other suitable manner.

In a variation, the characterization process can be generated and trained according to a random forest predictor (RFP) algorithm that combines bagging (e.g., bootstrap aggregation) and selection of random sets of features from a training dataset to construct a set of decision trees, T, associated with the random sets of features. In using a random forest algorithm, N cases from the set of decision trees are sampled at random with replacement to create a subset of decision trees, and for each node, m prediction features are selected from all of the prediction features for assessment. The prediction feature that provides the best split at the node (e.g., according to an objective function) is used to perform the split (e.g., as a bifurcation at the node, as a trifurcation at the node). By sampling many times from a large dataset, the strength of the characterization process, in identifying features that are strong in predicting classifications can be increased substantially. In this variation, measures to prevent bias (e.g., sampling bias) and/or account for an amount of bias can be included during processing, such as to increase robustness of the model.

In a variation, Block S130 and/or other portions of embodiments of the method 100 can include applying computer-implemented rules (e.g., models, feature selection rules, etc.) to process population-level data, but can additionally or alternatively include applying computer-implemented rules to process microbiome-related data on a demographic characteristic-specific basis (e.g., subgroups sharing one or more demographic characteristics such as therapy regimens, dietary regimens, physical activity regimens, ethnicity, age, gender, weight, behaviors, etc.), condition-specific basis (e.g., subgroups exhibiting a specific appendix-related condition, a combination of appendix-related conditions, triggers for the appendix-related conditions, associated symptoms, etc.), a sample type-specific basis (e.g., applying different computer-implemented rules to process microbiome data derived from different collection sites; etc.), a user basis (e.g., different computer-implemented rules for different users; etc.) and/or any other suitable basis. As such, Block S130 can include assigning users from the population of users to one or more subgroups; and applying different computer-implemented rules for determining features (e.g., the set of feature types used; the types of characterization models generated from the features; etc.) for the different subgroups. However, applying computer-implemented rules can be performed in any suitable manner.

In another variation, Block S130 can include processing (e.g., generating, training, updating, executing, storing, etc.) one or more appendix-related characterization models (e.g., appendix-related condition models, therapy models, etc.) for one or more appendix-related conditions (e.g., for outputting characterizations for users describing user microbiome characteristics in relation to appendix-related conditions; therapy models for outputting therapy determinations for one or more appendix-related conditions; etc.). The characterization models preferably leverage microbiome features as inputs, and preferably output appendix-related characterizations and/or any suitable components thereof; but characterization models can use any suitable inputs to generate any suitable outputs. In an example, Block S130 can include transforming the supplementary data, the microbiome composition diversity features, and the microbiome functional diversity features, other microbiome features, outputs of appendix-related characterization models, and/or other suitable data into one or more characterization models (e.g., training an appendix-related characterization model based on the supplementary data and microbiome features; etc.) for one or more appendix-related conditions. In another example, the method 100 can include: determining a population microorganism sequence dataset (e.g., including microorganism sequence outputs for different users of the population; etc.) for a population of users associated with one or more appendix-related conditions, based on a set of samples from the population of users (e.g., and/or based on one or more primer types associated with the appendix-related condition; etc.); collecting a supplementary dataset associated with diagnosis of the one or more appendix-related conditions for the population of subjects; and generating the appendix-related characterization model based on the population microorganism sequence dataset and the supplementary dataset. In an example, the method 100 can include determining a set of user microbiome features for the user based on a sample from the user, where the set of user microbiome features is associated with microbiome features associated with a set of subjects (e.g., microbiome features determined to be correlated with one or more appendix-related conditions, based on processing biological samples corresponding to a set of subjects associated with the one or more appendix-related conditions; a set microbiome composition features and the set of microbiome functional features; etc.); determining an appendix-related characterization, including determining a therapy for the user for the one or more appendix-related conditions based on a therapy model and the set of user microbiome features; providing the therapy (e.g., providing a recommendation for the therapy to the user at a computing device associated with the user, etc.) and/or otherwise facilitating therapeutic intervention.

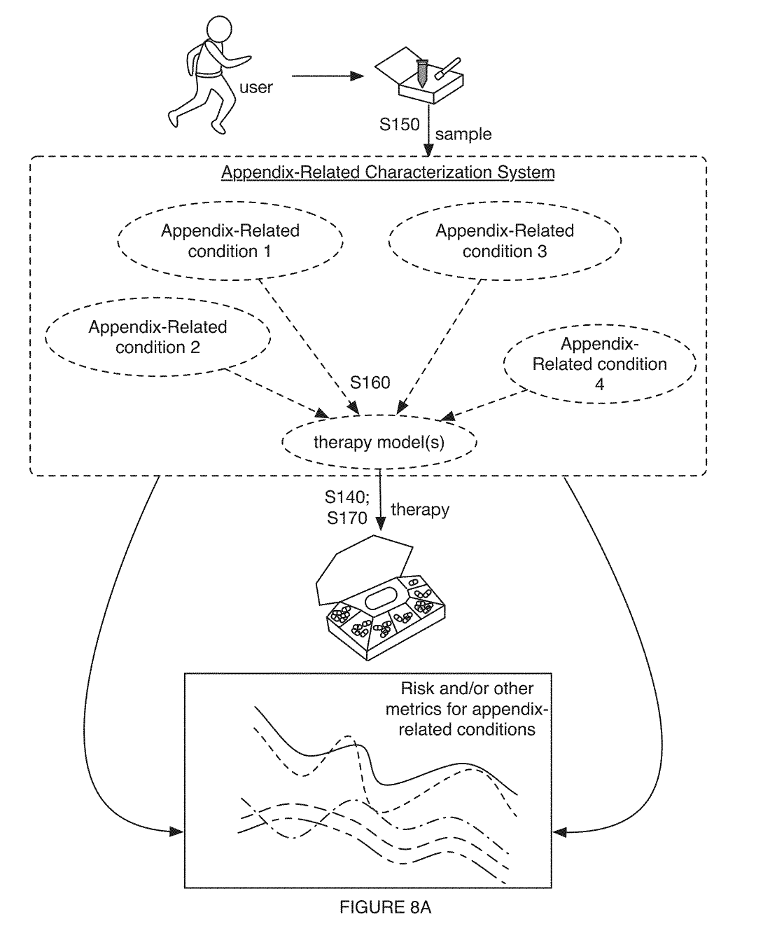

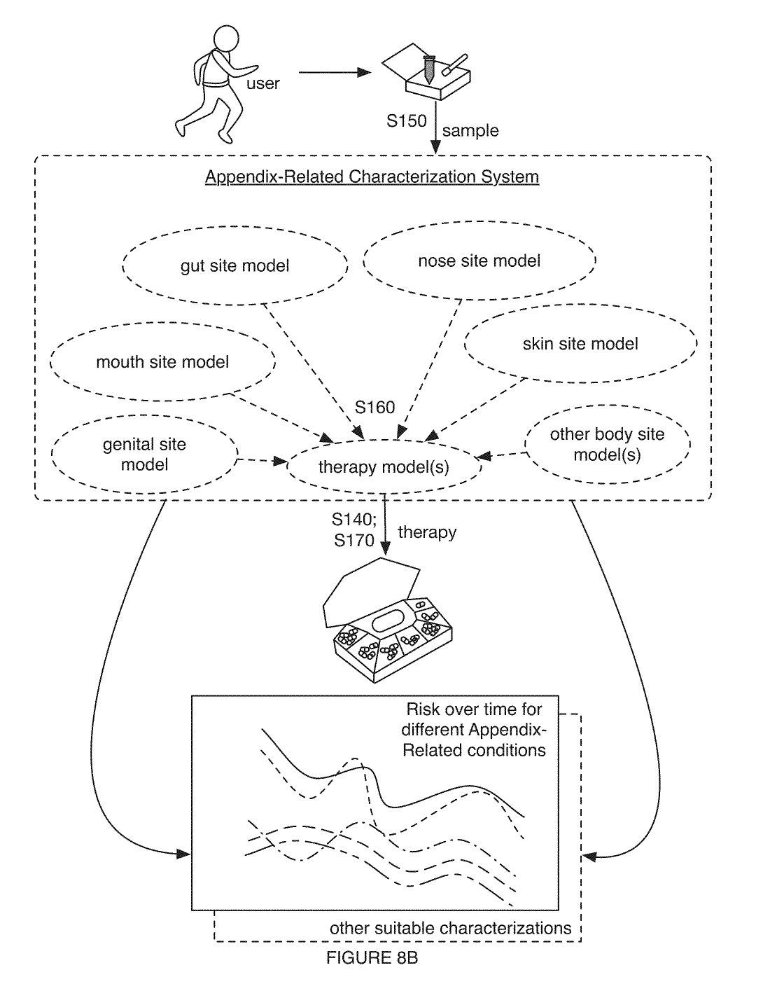

In another variation, as shown in FIGS. 8A-8B, different appendix-related characterization models and/or other suitable models (e.g., generated with different algorithms, with different sets of features, with different input and/or output types, applied in different manners such as in relation to time, frequency, component applying the model, etc.) can be generated for different appendix-related conditions, different user demographic characteristics (e.g., based on age, gender, weight, height, ethnicity; etc.), different body sites (e.g., a gut site model, a nose site model, a skin site model, a mouth site model, a genitals site model, etc.), individual users, supplementary data (e.g., models incorporating prior knowledge of microbiome features, appendix-related conditions, and/or other suitable components; features associated with biometric sensor data and/or survey response data vs. models independent of supplementary data, etc.), and/or other suitable criteria. In an specific example, the method 100 can include collecting first site-specific samples associated with a first body site (e.g., a gut site; samples collected by users at body collection sites corresponding to the first body site; one or more suitable body sites; etc.); determining a microorganism dataset based on the site-specific samples; determining first site-specific microbiome features (e.g., site-specific composition features; site-specific functional features; suitable microbiome features described herein in relation to appendix-related conditions; features associated with the first body site; etc.) based on the microorganism dataset; determining a first site-specific appendix-related characterization model (e.g., a gut site-specific appendix-related characterization model; etc.) based on the first site-specific microbiome features; and determining an appendix-related condition for a user for the appendix-related condition based on the first site-specific appendix-related characterization model (e.g., using the first site-specific appendix-related characterization model to process user microbiome features, such as user site-specific microbiome features, derived based on a user sample collected at a body collection site of the user corresponding to the first body site; etc.). In a specific example, the method 100 can include collecting second site-specific samples associated with a second body site (e.g., at least one of a skin site, a genital site, a mouth site, and a nose site; one or more suitable body sites; etc.); determining second site-specific microbiome features (e.g. site-specific composition features; site-specific functional features; features associated with the second body site; etc.); generating a second site-specific appendix-related characterization model (e.g., associated with the second body site; etc.) based on the second site-specific composition features; collecting a user sample from an additional user, the user sample associated with the second body site (e.g., collected by the additional user at a collection site corresponding to the second body site; etc.); and determining an additional appendix-related characterization for the additional user for the appendix-related condition based on the second site-specific appendix-related characterization model (e.g., selecting the second site-specific appendix-related characterization model, from a set of site-specific appendix-related characterization models, to apply based on the association between the user sample and the body site, such as selecting a skin site-specific appendix-related characterization model to apply based on a user sample being collected from a skin collection site of the user; etc.).

In variations, determining appendix-related characterizations and/or any other suitable characterizations can include determining site-specific appendix-related characterizations (e.g., site-specific analyses) including appendix-related characterizations in relation to specific body sites (e.g., gut, healthy gut, skin, nose, mouth, genitals, other suitable body sites, other sample collection sites, etc.), such as through any one or more of: determining an appendix-related characterization based on an appendix-related characterization model derived based on site-specific data (e.g., defining correlations between an appendix-related condition and microbiome features associated with one or more body sites); determining an appendix-related characterization based on a user biological sample collected at one or more body sites, and/or any other suitable site-related processes. In examples, machine learning approaches (e.g., classifiers, deep learning algorithms, SVM, random forest), parameter optimization approaches (e.g., Bayesian Parameter Optimization), validation approaches (e.g., cross validation approaches), statistical tests (e.g., univariate statistical techniques, multivariate statistical techniques, correlation analysis such as canonical correlation analysis, etc.), dimension reduction techniques (e.g., PCA), and/or other suitable analytical techniques (e.g., described herein) can be applied in determining site-related (e.g., body site-related, etc.) characterizations (e.g., using a one or more approaches for one or more sample collection sites, such as for each type of sample collection site, etc.), other suitable characterizations, therapies, and/or any other suitable outputs. In a specific example, performing a characterization process (e.g., determining an appendix-related characterization; determining microbiome features; based on an appendix-related characterization model; etc.) can include applying at least one of: machine learning approaches, parameter optimization approaches, statistical tests, dimension reduction approaches, and/or other suitable approaches (e.g., where microbiome features such as a set of microbiome composition diversity features and/or a set of microbiome functional diversity features can be associated with microorganisms collected at least at one of a gut site, a skin site, a nose site, a mouth site, a genitals site, etc.). In another specific example, characterization processes performed for a plurality of sample collection sites can be used to generate individual characterizations that can be combined to determine an aggregate characterization (e.g., an aggregate microbiome score, such as for one or more conditions described herein, etc.). However, the method 100 can include determining any suitable site-related (e.g., site-specific) outputs, and/or performing any suitable portions of embodiments of the method 100 (e.g., collecting samples, processing samples, determining therapies) with site-specificity and/or other site-relatedness in any suitable manner.

Characterization of the subject(s) can additionally or alternatively implement use of a high false positive test and/or a high false negative test to further analyze sensitivity of the characterization process in supporting analyses generated according to embodiments of the method 100. However, performing one or more characterization processes S130 can be performed in any suitable manner.

### 4.3.A Appendix-Related Characterization Process.

Performing a characterization process S130 can include performing an appendix-related characterization process (e.g., determining a characterization for one or more appendix-related conditions; determining and/or applying one or more appendix-related characterization models; etc.) S135, such as for one or more users (e.g., for data corresponding to samples from a set of subjects for generating one or more appendix-related characterization models, such as where one or more subjects are associated with the appendix-related conditions, such as subjects diagnosed with the one or more appendix-related conditions; for a single user for generating an appendix-related characterization for the user, such as through using one or more appendix-related characterization models, such as through applying the one or more appendix-related characterization models to a user microbiome sequence dataset derived from sequencing a sample from the user; etc.) and/or for one or more appendix-related conditions.

In variations, performing an appendix-related characterization process can include determining microbiome features associated with one or more appendix-related conditions. In an example, performing an appendix-related characterization process can include applying one or more analytical techniques (e.g., statistical analyses) to identify the sets of microbiome features (e.g., microbiome composition features, microbiome composition diversity features, microbiome functional features, microbiome functional diversity features, etc.) that have the highest correlations (e.g., positive correlations, negative correlations, etc.) with one or more appendix-related conditions (e.g., features associated with a single appendix-related condition, cross-condition features associated with multiple appendix-related conditions and/or other suitable appendix-related conditions, etc.). In a specific example, determining a set of microbiome features (e.g., correlated with and/or otherwise associated with one or more appendix-related conditions; for use in generating one or more appendix-related characterization models; etc.) can include applying a set of analytical techniques to determine at least one of presence of at least one of a microbiome composition diversity feature and a microbiome functional diversity feature, absence of the at least one of the microbiome composition diversity feature and the microbiome functional diversity feature, a relative abundance feature describing relative abundance of different taxonomic groups associated with the appendix-related condition, a ratio feature describing a ratio between at least two microbiome features associated with the different taxonomic groups, an interaction feature describing an interaction between the different taxonomic groups, and a phylogenetic distance feature describing phylogenetic distance between the different taxonomic groups, based on the microorganism sequence dataset, and/or where the set of analytical techniques can include at least one of a univariate statistical test, a multivariate statistical test, a dimensionality reduction technique, and an artificial intelligence approach.

In a specific example, performing an appendix-related characterization process can facilitate therapeutic intervention for one or more appendix-related conditions, such as through facilitating intervention associated with therapies having a positive effect on a state of one or more users in relation to the one or more appendix-related conditions. In another specific example, performing an appendix-related characterization process (e.g., determining features with highest correlations to one or more appendix-related conditions, etc.) can be based upon a random forest predictor algorithm trained with a training dataset derived from a subset of the population of subjects (e.g., subjects having the one or more appendix-related conditions; subjects not having the one or more appendix-related conditions; etc.), and validated with a validation dataset derived from a subset of the population of subjects. However, determining microbiome features and/or other suitable aspects associated with one or more appendix-related conditions can be performed in any suitable manner.

In variations, performing an appendix-related characterization process can include performing an appendix-related characterization process for an appendix-related condition including an absence of an appendix (e.g., a removed appendix; etc.) and/or inflammation associated with the appendix (e.g., appendicitis; an inflammatory condition of a large intestine portion proximal to an appendix region; etc.). In examples, performing the appendix-related characterization process can include identifying microbiome features correlated with (e.g., having the highest correlations; positively correlated; negatively correlated; etc.) with absence of an appendix and/or inflammation associated with the appendix (and/or other suitable appendix-related conditions, such as appendix-related conditions where one or more therapies would have a positive effect), such as based upon a random forest predictor algorithm trained with a training dataset derived from a subset of the population of subjects, and validated with a validation dataset derived from a subset of the population of subjects. In specific examples, appendix-related conditions can include conditions characterizable (e.g., diagnosable, etc.) by one or more of blood tests, urinary test, diagnostic imaging tests (e.g., ultrasound, CT scans, etc.), and/or other suitable diagnostic procedures (e.g., described herein, etc.).

Microbiome features (e.g., microbiome composition features; site-specific composition features associated with one or more body sites; microbiome functional features; site-specific functional features associated with one or more body sites; etc.) associated with one or more appendix-related conditions (e.g., positively correlated with; negatively correlated with; useful for diagnosis; etc.) can include features (e.g., microbiome composition features, etc.) associated with any combination of one or more of the following taxa (e.g., features describing abundance of; features describing relative abundance of; features describing functional aspects associated with; features derived from; features describing presence and/or absence of; etc.), such as in relation to one or more body sites (e.g., where microbiome composition features can include site-specific composition features associated with the one or more body sites, such as where correlations between the composition features and the one or more appendix-related conditions can be specific to the one or more body sites, such as specific to microbiome composition observed at the body site from samples collected at a body collection site corresponding to the body site; etc.): Gemella (genus) (e.g., genital site), Veillonella atypica (species) (e.g., genital site), Dialister pneumosintes (species) (e.g., genital site), Lactobacillus crispatus (species) (e.g., genital site), Phyllobacteriaceae (family) (e.g., genital site), Aquabacterium (genus) (e.g., genital site), Anaeroglobus (genus) (e.g., genital site), Anaeroglobus geminatus (species) (e.g., genital site), Ochrobactrum (genus) (e.g., genital site), Mobiluncus curtisii (species) (e.g., genital site), Actinomyces neuii (species) (e.g., genital site), Anaerococcus lactolyticus (species) (e.g., genital site), Lactobacillus johnsonii (species) (e.g., genital site), Verrucomicrobiales (order) (e.g., genital site), Verrucomicrobia (phylum) (e.g., genital site), Verrucomicrobiae (class) (e.g., genital site), Verrucomicrobiaceae (family) (e.g., genital site), Dialister succinatiphilus (species) (e.g., genital site), Atopobium sp. F0209 (species) (e.g., genital site), Corynebacterium freiburgense (species) (e.g., genital site), Lactobacillus sp. Akhmrol (species) (e.g., genital site), Anaerococcus sp. 9401487 (species) (e.g., genital site), Mesorhizobium (genus) (e.g., genital site), Lactobacillus reuteri (species) (e.g., genital site), Megasphaera sp. UPII 199-6 (species) (e.g., genital site), Lactobacillus sp. C30An8 (species) (e.g., genital site), Peptococcus sp. S9 Pr-12 (species) (e.g., genital site), Helcococcus seattlensis (species) (e.g., genital site), Neisseriaceae (family) (e.g., gut site), Neisseria mucosa (species) (e.g., gut site), Aggregatibacter aphrophilus (species) (e.g., gut site), Bacteroides uniformis (species) (e.g., gut site), Bacteroides vulgatus (species) (e.g., gut site), Parabacteroides distasonis (species) (e.g., gut site), Megasphaera (genus) (e.g., gut site), Proteobacteria (phylum) (e.g., gut site), Micrococcaceae (family) (e.g., gut site), Streptococcus thermophilus (species) (e.g., gut site), Streptococcus parasanguinis (species) (e.g., gut site), Gemella (genus) (e.g., gut site), Clostridium (genus) (e.g., gut site), Actinomyces odontolyticus (species) (e.g., gut site), Actinomycetales (order) (e.g., gut site), Actinomycetaceae (family) (e.g., gut site), Betaproteobacteria (class) (e.g., gut site), Gemella morbillorum (species) (e.g., gut site), Rothia (genus) (e.g., gut site), Lactobacillus crispatus (species) (e.g., gut site), Pseudomonadales (order) (e.g., gut site), Oxalobacteraceae (family) (e.g., gut site), Burkholderiales (order) (e.g., gut site), Gemella sp. 933-88 (species) (e.g., gut site), Micrococcales (order) (e.g., gut site), Bacteroides acidifaciens (species) (e.g., gut site), Mogibacterium (genus) (e.g., gut site), Bacteroides sp. AR20 (species) (e.g., gut site), Bacteroides sp. AR29 (species) (e.g., gut site), Burkholderiaceae (family) (e.g., gut site), Erysipelotrichaceae (family) (e.g., gut site), Xanthomonadales (order) (e.g., gut site), Pseudomonadaceae (family) (e.g., gut site), Actinomyces sp. oral strain Hal-1065 (species) (e.g., gut site), Roseburia intestinalis (species) (e.g., gut site), Porphyromonadaceae (family) (e.g., gut site), Shuttleworthia (genus) (e.g., gut site), Clostridia (class) (e.g., gut site), Clostridiales (order) (e.g., gut site), Peptostreptococcaceae (family) (e.g., gut site), Peptococcaceae (family) (e.g., gut site), Carnobacteriaceae (family) (e.g., gut site), Dialister sp. E2_20 (species) (e.g., gut site), Neisseriales (order) (e.g., gut site), Megasphaera genomosp. Ci (species) (e.g., gut site), Moryella (genus) (e.g., gut site), Synergistetes (phylum) (e.g., gut site), Erysipelotrichia (class) (e.g., gut site), Erysipelotrichales (order) (e.g., gut site), Clostridiales Family XIII. Incertae Sedis (family) (e.g., gut site), Roseburia sp. 11SE39 (species) (e.g., gut site), Bacteroides sp. D22 (species) (e.g., gut site), Synergistia (class) (e.g., gut site), Synergistales (order) (e.g., gut site), Synergistaceae (family) (e.g., gut site), Lactobacillus sp. TAB-22 (species) (e.g., gut site), Flavonifractor (genus) (e.g., gut site), Sutterellaceae (family) (e.g., gut site), Anaerostipes sp. 5_1_63FAA (species) (e.g., gut site), Streptococcus sp. 2011_Oral_MS_A3 (species) (e.g., gut site), Veillonella sp. 2011_Oral_VSA_D3 (species) (e.g., gut site), Finegoldia sp. S9 AA1-5 (species) (e.g., gut site), Fretibacterium (genus) (e.g., gut site), Staphylococcus sp. 334802 (species) (e.g., gut site), Peptoclostridium (genus) (e.g., gut site), Intestinibacter (genus) (e.g., gut site), Acinetobacter (genus) (e.g., gut site), Klebsiella (genus) (e.g., gut site), Bacteroides thetaiotaomicron (species) (e.g., gut site), Butyrivibrio (genus) (e.g., gut site), Fusobacterium necrogenes (species) (e.g., gut site), Herbaspirillum (genus) (e.g., gut site), Herbaspirillum seropedicae (species) (e.g., gut site), Pediococcus (genus) (e.g., gut site), Finegoldia magna (species) (e.g., gut site), Blautia hansenii (species) (e.g., gut site), Enterococcus faecalis (species) (e.g., gut site), Lactococcus lactis (species) (e.g., gut site), Bacillus (genus) (e.g., gut site), Clostridioides difficile (species) (e.g., gut site), Blautia coccoides (species) (e.g., gut site), Erysipelatoclostridium ramosum (species) (e.g., gut site), Weissella confusa (species) (e.g., gut site), Lactobacillus plantarum (species) (e.g., gut site), Lactobacillus paracasei (species) (e.g., gut site), Bifidobacterium adolescentis (species) (e.g., gut site), Bifidobacterium breve (species) (e.g., gut site), Bifidobacterium dentium (species) (e.g., gut site), Bifidobacterium animalis (species) (e.g., gut site), Bifidobacterium pseudocatenulatum (species) (e.g., gut site), Bacteroides ovatus (species) (e.g., gut site), Peptoniphilus lacrimalis (species) (e.g., gut site), Anaerococcus vaginalis (species) (e.g., gut site), Rahnella (genus) (e.g., gut site), Bilophila wadsworthia (species) (e.g., gut site), Sneathia sanguinegens (species) (e.g., gut site), Succiniclasticum (genus) (e.g., gut site), Sporobacter (genus) (e.g., gut site), Pseudobutyrivibrio ruminis (species) (e.g., gut site), Weissella (genus) (e.g., gut site), Bacteroides stercoris (species) (e.g., gut site), Lactobacillus rhamnosus (species) (e.g., gut site), Pantoea (genus) (e.g., gut site), Holdemania (genus) (e.g., gut site), Holdemania filiformis (species) (e.g., gut site), Thermoanaerobacterales (order) (e.g., gut site), Bifidobacterium gallicum (species) (e.g., gut site), Bifidobacterium pullorum (species) (e.g., gut site), Leuconostocaceae (family) (e.g., gut site), Eggerthella lenta (species) (e.g., gut site), Papillibacter (genus) (e.g., gut site), Anaerostipes caccae (species) (e.g., gut site), Pseudoflavonifractor capillosus (species) (e.g., gut site), Anaerovorax (genus) (e.g., gut site), Parasporobacterium (genus) (e.g., gut site), Parasporobacterium paucivorans (species) (e.g., gut site), Oscillospira (genus) (e.g., gut site), Oscillospira guilliermondii (species) (e.g., gut site), Actinomyces turicensis (species) (e.g., gut site), Anaerosinus (genus) (e.g., gut site), Sneathia (genus) (e.g., gut site), Brevibacterium paucivorans (species) (e.g., gut site), Lactobacillus sp. CR-609S (species) (e.g., gut site), Thermoanaerobacteraceae (family) (e.g., gut site), Bacillaceae (family) (e.g., gut site), Gelria (genus) (e.g., gut site), Acidobacteriales (order) (e.g., gut site), Bacteroides massiliensis (species) (e.g., gut site), Rhodocyclales (order) (e.g., gut site), Anaerofustis stercorihominis (species) (e.g., gut site), Alistipes finegoldii (species) (e.g., gut site), Oscillospiraceae (family) (e.g., gut site), Peptoniphilus sp. 2002-38328 (species) (e.g., gut site), Hespellia (genus) (e.g., gut site), Bacteroides sp. 35AE37 (species) (e.g., gut site), Marvinbryantia (genus) (e.g., gut site), Anaerosporobacter mobilis (species) (e.g., gut site), Anaerofustis (genus) (e.g., gut site), Catabacter (genus) (e.g., gut site), Flavonifractor plautii (species) (e.g., gut site), Proteiniphilum (genus) (e.g., gut site), Roseburia faecis (species) (e.g., gut site), Streptococcus sp. S16-11 (species) (e.g., gut site), Bacteroides sp. 4072 (species) (e.g., gut site), Alistipes shahii (species) (e.g., gut site), Bacteroides intestinalis (species) (e.g., gut site), Lactonifactor longoviformis (species) (e.g., gut site), Bifidobacterium tsurumiense (species) (e.g., gut site), Bacteroides dorei (species) (e.g., gut site), Bacteroides xylanisolvens (species) (e.g., gut site), Cronobacter (genus) (e.g., gut site), Alloscardovia (genus) (e.g., gut site), Alloscardovia omnicolens (species) (e.g., gut site), Lactonifactor (genus) (e.g., gut site), Catabacteriaceae (family) (e.g., gut site), Adlercreutzia equolifaciens (species) (e.g., gut site), Adlercreutzia (genus) (e.g., gut site), Alistipes sp. EBA6-25c12 (species) (e.g., gut site), Bacteroides sp. EBA5-17 (species) (e.g., gut site), Oscillibacter (genus) (e.g., gut site), Gordonibacter pamelaeae (species) (e.g., gut site), Alistipes sp. NML05A004 (species) (e.g., gut site), Parasutterella excrementihominis (species) (e.g., gut site), Mitsuokella sp. DJF_RR21 (species) (e.g., gut site), Butyricimonas (genus) (e.g., gut site), Bifidobacterium stercoris (species) (e.g., gut site), Alistipes indistinctus (species) (e.g., gut site), Gordonibacter (genus) (e.g., gut site), Anaerostipes hadrus (species) (e.g., gut site), Klebsiella sp. B12 (species) (e.g., gut site), Alistipes sp. RMA 9912 (species) (e.g., gut site), Anaerosporobacter (genus) (e.g., gut site), Bacteroides faecis (species) (e.g., gut site), Blautia sp. Ser5 (species) (e.g., gut site), Bacteroides chinchillae (species) (e.g., gut site), Bilophila sp. 4_1_30 (species) (e.g., gut site), Caldicoprobacteraceae (family) (e.g., gut site), Enterobacter sp. UDC345 (species) (e.g., gut site), Bifidobacterium biavatii (species) (e.g., gut site), Peptoniphilus sp. 1-14 (species) (e.g., gut site), Alistipes sp. HGB5 (species) (e.g., gut site), Bacteroides sp. SLC1-38 (species) (e.g., gut site), Lactobacillus sp. Akhmrol (species) (e.g., gut site), Klebsiella sp. SOR89 (species) (e.g., gut site), Enterococcus sp. C6 I11 (species) (e.g., gut site), Pseudoflavonifractor (genus) (e.g., gut site), Bacteroides sp. dnLKV9 (species) (e.g., gut site), Megasphaera sp. BV3C16-1 (species) (e.g., gut site), Faecalibacterium sp. canine oral taxon 147 (species) (e.g., gut site), Varibaculum sp. CCUG 45114 (species) (e.g., gut site), Butyricimonas sp. 214-4 (species) (e.g., gut site), Anaerostipes rhamnosivorans (species) (e.g., gut site), Negativicoccus sp. S5-A15 (species) (e.g., gut site), [Collinsella] massiliensis (species) (e.g., gut site), Corynebacterium sp. jw37 (species) (e.g., gut site), Roseburia sp. 499 (species) (e.g., gut site), Dialister sp. S7MSR5 (species) (e.g., gut site), Anaerococcus sp. S8 87-3 (species) (e.g., gut site), Finegoldia sp. S8 F7 (species) (e.g., gut site), Murdochiella sp. S9 PR-10 (species) (e.g., gut site), Peptoniphilus sp. S9 PR-13 (species) (e.g., gut site), Bacteroides sp. J1511 (species) (e.g., gut site), Corynebacterium sp. 713182/2012 (species) (e.g., gut site), Rahnella sp. BSP18 (species) (e.g., gut site), Intestinimonas (genus) (e.g., gut site), Robinsoniella sp. KNHs210 (species) (e.g., gut site), Candidatus Soleaferrea (genus) (e.g., gut site), Butyricimonas faecihominis (species) (e.g., gut site), Senegalimassilia (genus) (e.g., gut site), Peptoniphilus sp. DNF00840 (species) (e.g., gut site), Romboutsia (genus) (e.g., gut site), Coprobacter secundus (species) (e.g., gut site), Moraxellaceae (family) (e.g., mouth site), Moraxella (genus) (e.g., mouth site), Eikenella (genus) (e.g., mouth site), Eikenella corrodens (species) (e.g., mouth site), Vagococcus (genus) (e.g., mouth site), Phyllobacterium (genus) (e.g., mouth site), Veillonella dispar (species) (e.g., mouth site), Sutterella wadsworthensis (species) (e.g., mouth site), Johnsonella ignava (species) (e.g., mouth site), Bacteroides acidifaciens (species) (e.g., mouth site), Leptotrichia hofstadii (species) (e.g., mouth site), Leptotrichia shahii (species) (e.g., mouth site), Capnocytophaga sp. AHN9756 (species) (e.g., mouth site), Bergeyella sp. AF14 (species) (e.g., mouth site), Olsenella sp. F0004 (species) (e.g., mouth site), Bacteroides sp. D22 (species) (e.g., mouth site), Phyllobacterium sp. T50 (species) (e.g., mouth site), Actinomyces sp. ICM47 (species) (e.g., mouth site), Fusobacterium sp. AS2 (species) (e.g., mouth site), Leptotrichiaceae (family) (e.g., mouth site), Comamonas (genus) (e.g., nose site), Peptostreptococcus (genus) (e.g., nose site), Actinomyces viscosus (species) (e.g., nose site), Actinomyces odontolyticus (species) (e.g., nose site), Bifidobacterium (genus) (e.g., nose site), Bifidobacteriaceae (family) (e.g., nose site), Rhodospirillaceae (family) (e.g., nose site), Bifidobacteriales (order) (e.g., nose site), Roseburia intestinalis (species) (e.g., nose site), Thalassospira (genus) (e.g., nose site), Bifidobacterium longum (species) (e.g., nose site), Aggregatibacter (genus) (e.g., nose site), Streptococcus sp. 11aTh1 (species) (e.g., nose site), Sutterellaceae (family) (e.g., nose site), Flavobacterium (genus) (e.g., nose site), Ochrobactrum (genus) (e.g., nose site), Cronobacter sakazakii (species) (e.g., nose site), Anaerococcus vaginalis (species) (e.g., nose site), Sphingobacteriia (class) (e.g., nose site), Brucellaceae (family) (e.g., nose site), Sphingobacteriales (order) (e.g., nose site), Akkermansia (genus) (e.g., nose site), Peptoniphilus sp. gpac018A (species) (e.g., nose site), Citrobacter sp. BW4 (species) (e.g., nose site), Cronobacter (genus) (e.g., nose site), Corynebacterium sp. jw37 (species) (e.g., nose site), Staphylococcus aureus (species) (e.g., nose site), Brevundimonas (genus) (e.g., nose site), Caulobacteraceae (family) (e.g., nose site), Caulobacterales (order) (e.g., nose site), Anaerobacillus alkalidiazotrophicus (species) (e.g., nose site), Anaerobacillus (genus) (e.g., nose site), Acinetobacter sp. WB22-23 (species) (e.g., nose site), Pseudomonas (genus) (e.g., skin site), Neisseriaceae (family) (e.g., skin site), Parabacteroides distasonis (species) (e.g., skin site), Prevotella (genus) (e.g., skin site), Faecalibacterium prausnitzii (species) (e.g., skin site), Streptococcus parasanguinis (species) (e.g., skin site), Cutibacterium acnes (species) (e.g., skin site), Veillonellaceae (family) (e.g., skin site), Leptotrichia (genus) (e.g., skin site), Phascolarctobacterium (genus) (e.g., skin site), Flavobacteriaceae (family) (e.g., skin site), Delftia (genus) (e.g., skin site), Flavobacteriia (class) (e.g., skin site), Prevotellaceae (family) (e.g., skin site), Lachnospiraceae (family) (e.g., skin site), Peptostreptococcaceae (family) (e.g., skin site), Dorea (genus) (e.g., skin site), Flavobacteriales (order) (e.g., skin site), Neisseriales (order) (e.g., skin site), Parabacteroides (genus) (e.g., skin site), Streptococcus sp. oral taxon G63 (species) (e.g., skin site), Acidaminococcaceae (family) (e.g., skin site), Veillonella sp. CM60 (species) (e.g., skin site), Staphylococcus sp. C912 (species) (e.g., skin site), Leptotrichiaceae (family) (e.g., skin site), Fusicatenibacter saccharivorans (species) (e.g., skin site), Fusicatenibacter (genus) (e.g., skin site), Staphylococcus sp. 334802 (species) (e.g., skin site), Parabacteroides merdae (species) (e.g., skin site), Collinsella aerofaciens (species) (e.g., skin site), Sphingobacteriia (class) (e.g., skin site), Sphingobacteriales (order) (e.g., skin site), Peptoniphilus sp. 1-14 (species) (e.g., skin site), Anaerobacillus (genus) (e.g., skin site), Propionibacterium sp. KPL1844 (species) (e.g., skin site), Methylobacterium longum (species) (e.g., skin site), Staphylococcus sp. C5116 (species) (e.g., skin site), and/or other suitable taxa (e.g., associated with any suitable body sites, etc.).

Additionally or alternatively, microbiome features associated with one or more appendix-related conditions can include features (e.g., microbiome compostion features; etc.) associated with any combination of one or more of the following taxa (e.g., such as in relation to one or more body sites, etc.): Firmicutes (phylum), Enterococcus raffinosus (species), Staphylococcus sp. C912 (species), Gemella sp. 933-88 (species), Veillonella (genus), Gammaproteobacteria (class), Enterococcus sp. SI-4 (species), Enterobacteriales (order), Enterobacteriaceae (family), Phascolarctobacterium (genus), Odoribacter (genus), Ruminococcaceae (family), Acidaminococcaceae (family), Bilophila sp. 4_1_30 (species), Anaerostipes sp. 5_1_63FAA (species), Desulfovibrionaceae (family), Phascolarctobacterium faecium (species), Desulfovibrionales (order), Faecalibacterium (genus), Deltaproteobacteria (class), Burkholderiaceae (family), Alistipes sp. RMA 9912 (species), Methanobrevibacter (genus), Odoribacter splanchnicus (species), Alistipes sp. HGB5 (species), Gemella (genus), Subdoligranulum variabile (species), Methanobrevibacter smithii (species), Intestinimonas (genus), Lactobacillus sp. 7_1_47FAA (species), Methanobacteriaceae (family), Bilophila (genus), Methanobacteriales (order), Clostridiaceae (family), Euryarchaeota (phylum), Methanobacteria (class), Flavonifractor plautii (species), Carnobacteriaceae (family), Kluyvera (genus), Kluyvera georgiana (species), Blautia faecis (species), Faecalibacterium prausnitzii (species), Lactonifactor longoviformis (species), Roseburia sp. 11SE39 (species), Bacteroides sp. AR29 (species), Collinsella (genus), Alistipes sp. NML05A004 (species), Prevotella timonensis (species), Anaerostipes (genus), Lactonifactor (genus), Anaerostipes sp. 3_2_56FAA (species), Coriobacteriaceae (family), Klebsiella sp. SOR89 (species), Megasphaera sp. DNF00912 (species), Veillonella dispar (species), Lactobacillus mucosae (species), Bacteroides fragilis (species), Streptococcus equinus (species), Bacteroides plebeius (species), Propionibacterium sp. MSP09A (species), Streptococcus pasteurianus (species), Anaerovibrio sp. 765 (species), Akkermansia muciniphila (species), Actinomyces turicensis (species), Cronobacter sakazakii (species), Veillonella rogosae (species), Blautia glucerasea (species), Acidaminococcus intestini (species), Propionibacterium granulosum (species), Bacteroides thetaiotaomicron (species), Fusobacterium sp. CM21 (species), Pediococcus sp. MFC1 (species), Turicibacter sanguinis (species), Sarcina ventriculi (species), Megasphaera genomosp. C1 (species), Streptococcus sp. BS35a (species), Streptococcus thermophilus (species), Fusobacterium ulcerans (species), Morganella morganii (species), Bacteroides sp. SLC1-38 (species), Bacteroides eggerthii (species), Bacteroides coprocola (species), Bacteroides sp. CB57 (species), Bifidobacterium stercoris (species), Veillonella atypica (species), Fusobacterium necrogenes (species), Lactobacillus crispatus (species), Veillonella sp. MSA12 (species), Asaccharospora irregularis (species), Erysipelatoclostridium ramosum (species), Lactobacillus sp. TAB-22 (species), Parasutterella excrementihominis (species), Lactobacillus sp. C412 (species), Parabacteroides sp. 157 (species), Klebsiella (genus), Epulopiscium (genus), Streptococcus (genus), Propionibacterium (genus), Cronobacter (genus), Anaerovibrio (genus), Intestinibacter (genus), Staphylococcus (genus), Turicibacter (genus), Alloprevotella (genus), Pediococcus (genus), Morganella (genus), Acidaminococcus (genus), Succinivibrio (genus), Anaerofilum (genus), Megasphaera (genus), Asaccharospora (genus), Butyrivibrio (genus), Finegoldia (genus), Anaerococcus (genus), Streptococcaceae (family), Propionibacteriaceae (family), Veillonellaceae (family), Staphylococcaceae (family), Sphingobacteriaceae (family), Clostridiales Family XI. Incertae Sedis (family), Peptostreptococcaceae (family), Succinivibrionaceae (family), Dermabacteraceae (family), Corynebacteriaceae (family), Rhodospirillaceae (family), Selenomonadales (order), Lactobacillales (order), Clostridiales (order), Xanthomonadales (order), Bacillales (order), Pleurocapsales (order), Aeromonadales (order), Pseudomonadales (order), Bacilli (class), Negativicutes (class), Clostridia (class), Proteobacteria (phylum), Cyanobacteria (phylum), Bacteroides finegoldii (species), Alistipes putredinis (species), Actinobacteria (class), Lactobacillaceae (family), Bifidobacteriaceae (family), Bifidobacterium (genus), Bifidobacteriales (order), Oscillospiraceae (family), and/or other suitable taxa (e.g., associated with any suitable body sites, etc.).

Additionally or alternatively, microbiome features associated with one or more appendix-related conditions can include microbiome functional features (e.g., features describing functions associated with one or more microorganisms, such as microorganisms classified under taxa described herein; features describing functional diversity; features describing presence, absence, abundance, and/or relative abundance; etc.) corresponding to functions from and/or otherwise associated with (e.g., such as in relation to one or more body sites, where microbiome functional features can include site-specific functional features associated with the one or more body sites, such as where correlations between the functional features and the one or more appendix-related conditions can be specific to the body site, such as specific to microbiome function corresponding to microorganisms observed at the body site from samples collected at a body collection site corresponding to the body site; etc.) one or more of: Neurodegenerative Diseases (e.g., KEGG Pathways Level 2) (e.g., gut site), Signaling Molecules and Interaction (e.g., KEGG Pathways Level 2) (e.g., gut site), Xenobiotics Biodegradation and Metabolism (e.g., KEGG Pathways Level 2) (e.g., gut site), Ascorbate and aldarate metabolism (e.g., KEGG Pathways Level 3) (e.g., gut site), Huntington's disease (e.g., KEGG Pathways Level 3) (e.g., gut site), Inositol phosphate metabolism (e.g., KEGG Pathways Level 3) (e.g., gut site), Propanoate metabolism (e.g., KEGG Pathways Level 3) (e.g., gut site), Starch and sucrose metabolism (e.g., KEGG Pathways Level 3) (e.g., gut site), Caprolactam degradation (e.g., KEGG Pathways Level 3) (e.g., gut site), Cell motility and secretion (e.g., KEGG Pathways Level 3) (e.g., gut site), Valine, leucine and isoleucine degradation (e.g., KEGG Pathways Level 3) (e.g., gut site), Tryptophan metabolism (e.g., KEGG Pathways Level 3) (e.g., gut site), Type I diabetes mellitus (e.g., KEGG Pathways Level 3) (e.g., gut site), Phenylalanine metabolism (e.g., KEGG Pathways Level 3) (e.g., gut site), Selenocompound metabolism (e.g., KEGG Pathways Level 3) (e.g., gut site), Lysine degradation (e.g., KEGG Pathways Level 3) (e.g., gut site), Polycyclic aromatic hydrocarbon degradation (e.g., KEGG Pathways Level 3) (e.g., gut site), Glycan biosynthesis and metabolism (e.g., KEGG Pathways Level 3) (e.g., gut site), Renal cell carcinoma (e.g., KEGG Pathways Level 3) (e.g., gut site), Butanoate metabolism (e.g., KEGG Pathways Level 3) (e.g., gut site), Carbon fixation pathways in prokaryotes (e.g., KEGG Pathways Level 3) (e.g., gut site), Citrate cycle (TCA cycle) (e.g., KEGG Pathways Level 3) (e.g., gut site), Lipopolysaccharide biosynthesis (e.g., KEGG Pathways Level 3) (e.g., gut site), RNA transport (e.g., KEGG Pathways Level 3) (e.g., gut site), Thiamine metabolism (e.g., KEGG Pathways Level 3) (e.g., gut site), 1,1,1-Trichloro-2,2-bis(4-chlorophenyl)ethane (DDT) degradation (e.g., KEGG Pathways Level 3) (e.g., gut site), Electron transfer carriers (e.g., KEGG Pathways Level 3) (e.g., gut site), Amyotrophic lateral sclerosis (ALS) (e.g., KEGG Pathways Level 3) (e.g., gut site), Prion diseases (e.g., KEGG Pathways Level 3) (e.g., gut site), Toluene degradation (e.g., KEGG Pathways Level 3) (e.g., gut site), and alpha-Linolenic acid metabolism (e.g., KEGG Pathways Level 3) (e.g., gut site). Additionally or alternatively, microbiome features associated with one or more appendix-related conditions can include microbiome functional features corresponding to functions from and/or otherwise associated with one or more of: [V] Defense mechanisms (COG2), [O] Post-translational modification, protein turnover, and chaperones (COG2), [R] General function prediction only (COG2), [I] Lipid transport and metabolism (COG2), [H] Coenzyme transport and metabolism (COG2), Energy Metabolism (KEGG2), Nervous System (KEGG2), Signal Transduction (KEGG2), Cellular Processes and Signaling (KEGG2), Translation (KEGG2), Metabolism (KEGG2), Cell Growth and Death (KEGG2), Endocrine System (KEGG2), Amino Acid Metabolism (KEGG2), Metabolism of Cofactors and Vitamins (KEGG2), Xenobiotics Biodegradation and Metabolism (KEGG2), Replication and Repair (KEGG2), Metabolism of Terpenoids and Polyketides (KEGG2), Infectious Diseases (KEGG2), Amino acid related enzymes (KEGG3), Polycyclic aromatic hydrocarbon degradation (KEGG3), Photosynthesis (KEGG3), Pantothenate and CoA biosynthesis (KEGG3), Photosynthesis proteins (KEGG3), Glutamatergic synapse (KEGG3), Tuberculosis (KEGG3), Two-component system (KEGG3), Aminoacyl-tRNA biosynthesis (KEGG3), Thiamine metabolism (KEGG3), Ribosome (KEGG3), Other ion-coupled transporters (KEGG3), Terpenoid backbone biosynthesis (KEGG3), Cell cycle—Caulobacter (KEGG3), Other transporters (KEGG3), Base excision repair (KEGG3), Peptidoglycan biosynthesis (KEGG3), Vibrio cholerae pathogenic cycle (KEGG3), Limonene and pinene degradation (KEGG3), Secretion system (KEGG3), Nucleotide excision repair (KEGG3), Translation factors (KEGG3), Alanine, aspartate and glutamate metabolism (KEGG3), Ribosome Biogenesis (KEGG3), Butanoate metabolism (KEGG3), Others (KEGG3), Ribosome biogenesis in eukaryotes (KEGG3), Polyketide sugar unit biosynthesis (KEGG3), Streptomycin biosynthesis (KEGG3), Ascorbate and aldarate metabolism (KEGG3), Homologous recombination (KEGG3), Oxidative phosphorylation (KEGG3), Function unknown (KEGG3), Carbon fixation in photosynthetic organisms (KEGG3), Cytoskeleton proteins (KEGG3), DNA repair and recombination proteins (KEGG3), Lysine degradation (KEGG3), Inorganic ion transport and metabolism (KEGG3), Amino acid metabolism (KEGG3), Geraniol degradation (KEGG3), Protein export (KEGG3), Phenylalanine, tyrosine and tryptophan biosynthesis (KEGG3), Lysine biosynthesis (KEGG3), Ethylbenzene degradation (KEGG3), Transcription machinery (KEGG3), RNA polymerase (KEGG3), Biosynthesis of vancomycin group antibiotics (KEGG3), Mismatch repair (KEGG3), Naphthalene degradation (KEGG3), Pyrimidine metabolism (KEGG3), Tryptophan metabolism (KEGG3), D-Glutamine and D-glutamate metabolism (KEGG3), Zeatin biosynthesis (KEGG3), K02004 (KEGG4), K03100 (KEGG4), and/or other suitable functional features. Additionally or alternatively, microbiome functional features can be associated with any suitable functions described in relation to Clusters of Orthologous Groups (COG) databases (e.g., COG, COG2, etc.), Kyoto Encyclopedia of Genes and Genomes (KEGG) databases (e.g., KEGG2, KEGG3, KEGG4, etc.), and/or any other suitable database available (e.g., databases with microorganism function data, etc.). However, microbiome features can include any suitable microbiome functional features associated with any suitable microorganism function, human function, and/or other suitable functionality.

In variations, site-specific appendix-related characterization models (e.g., for determining appendix-related characterizations based on processing user site-specific microbiome features associated with one or more body sites also associated with the site-specific appendix-related characterization model; etc.) and/or appendix-related characterizations (e.g., associated with a body site, etc.) can be determined based on site-specific microbiome features (e.g., associated with one or more body sites; etc.) described herein (e.g., site-specific composition features; site-specific functional features; etc.). In examples, the method 100 can include determining user microbiome features (e.g., for a user for which an appendix-related characterization and/or therapy can be determined and/or promoted; determining feature values for a user for microbiome features determined to be associated with, such as correlated with, the one or more appendix-related conditions; etc.) including site-specific microbiome features associated with one or more body sites.

In variations, appendix-related characterization models and/or appendix-related characterizations can be determined based on microbiome features (e.g., associated with the one or more appendix-related conditions; etc.) including microbiome composition features (e.g., site-specific composition features; etc.) and microbiome functional features (e.g., site-specific functional features, etc.). In an example, the method 100 can include determining site-specific composition features (e.g., associated with a gut site; composition features described herein; etc.) and site-specific functional features (e.g., associated with a gut site; functional features described herein; etc.); and generating a site-specific appendix-related characterization model (e.g., associated with the gut site; for processing data derived from samples collected at gut collection sites; etc.) based on the site-specific composition features, the site-specific functional features, and/or other suitable data (e.g., supplementary data, etc.); and/or determining one or more appendix-related characterizations for one or more users based on the site-specific appendix-related characterization model and user microbiome features (e.g., derived from user samples collected at gut collection sites; etc.).

In specific examples, microbiome composition features (e.g., including site-specific composition features, etc.) described herein, microbiome functional features described herein, and/or other suitable microbiome features can be determined based on one or more microorganism datasets (e.g., microorganism sequence dataset, etc.) determined based on samples (e.g., sequencing of microorganism nucleic acids of the samples, etc.) from a set of subjects associated with the appendix-related condition (e.g., a set of subjects including subjects with the appendix-related condition such as an absence of an appendix and/or other suitable appendix-related conditions; including subjects without the appendix-related condition such as subjects with an appendix, where such samples and/or associated data can act as a control; a population of subjects; etc.).

In a variation, any suitable combination of microbiome features described herein can be used for an appendicitis characterization process (e.g., determining and/or applying appendicitis characterization model for performing diagnosis and/or suitable characterizations of an appendicitis condition; facilitating determination of and/or application of a therapy model and/or therapies for an appendicitis condition; etc.). In an example, a combination of microbiome feature can be predictive of the likelihood of appendicitis for an individual, based on his/her own gut microbiome sample, including presence, absence, relative abundance or any other microbiome features derived from gut samples analysis.

In variations, any suitable combination of microbiome features described herein can be used in prevention, treatment of, and/or suitable facilitation of therapeutic intervention for one or more appendix-related conditions associated with microorganisms, such as for restoring intestinal microbiota to a healthy cohort (e.g., improving microbiome diversity), such as including modulation of the presence, absence or relative abundance of microorganisms in a human gut microbiome and/or other suitable microbiomes associated with suitable body sites (e.g., towards a target microbiome composition and/or functionality associated with users with an appendix and without symptoms associated with inflammatory intestinal disease and/or with other suitable appendix-related conditions). However, microbiome features associated with appendix-related conditions can be applied in any suitable manner for prevention, treatment of, and/or suitable facilitation of therapeutic intervention for one or more appendix-related conditions.

In an example, the method 100 can include determining an appendix-related characterization for the user for a first appendix-related condition and a second appendix-related condition based on a first set of composition features (e.g., including at least one or more of the microbiome features described above in relation to the first variation; including any suitable combination of microbiome features; etc.), a first appendix-related characterization model, a second set of composition features (e.g., including at least one or more of the microbiome features described above in relation to the second variation; including any suitable combination of microbiome features; etc.), and a second appendix-related characterization model, where the first appendix-related characterization model is associated with the first appendix-related condition (e.g., where the first appendix-related characterization model determines characterizations for the first appendix-related condition, etc.), and where the second appendix-related characterization model is associated with the second appendix-related condition (e.g., where the second appendix-related characterization model determines characterizations for the second appendix-related condition, etc.). In the example, determining user microbiome features can include determining first user microbiome functional features associated with first functions from at least one of Cluster of Orthologous Groups (COG) database and Kyoto Encyclopedia of Genes and Genomes (KEGG) database, where the first user microbiome functional features are associated with the first appendix-related condition; and determining second user microbiome functional features associated with second functions from at least one of the COG database and the KEGG database, where the second user microbiome functional features are associated with the second appendix-related condition, where determining the appendix-related characterization can include determining the appendix-related characterization for the user for the first appendix-related condition and the second appendix-related condition based on the first set of composition features, the first user microbiome functional features, the first appendix-related characterization model, the second set of composition features, the second user microbiome functional features, and the second appendix-related characterization model. Additionally or alternatively, any combinations of microbiome features can be used with any suitable number and types of appendix-related characterization models to determine appendix-related characterization for one or more appendix-related conditions, in any suitable manner.

In examples, the method 100 can include generating one or more appendix-related characterization models based on any suitable combination of microbiome features described above and/or herein (e.g., based on a set of microbiome composition features including features associated with at least one of the taxa described herein; and/or based on microbiome functional features described herein, such as corresponding to functions from databases described herein; etc.) In an example, performing a characterization process for a user can include characterizing a user as having one or more appendix-related conditions, such as based upon detection of, values corresponding to, and/or other aspects related to microbiome features described herein (e.g., microbiome features described above, etc.), and such as in a manner that is an additional (e.g., supplemental to, complementary to, etc.) or alternative to typical approaches of diagnosis, other characterizations (e.g., treatment-related characterizations, etc.), treatment, monitoring, and/or other suitable approaches associated with appendix-related conditions. In variations, the microbiome features can be used for diagnostics, other characterizations, treatment, monitoring, and/or any other suitable purposes and/or approaches associated with appendix-related conditions. However, determining one or more appendix-related characterizations can be performed in any suitable manner.

### 4.3.B Determining a Therapy.

Performing a characterization process S130 (e.g., performing an appendix-related therapy) can include Block S140, which can include determining one or more therapies (e.g., therapies configured to modulate microbiome composition, function, diversity, and/or other suitable aspects, such as for improving one or more aspects associated with appendix-related conditions, such as in users characterized based on one or more characterization processes; etc.). Block S140 can function to identify, select, rank, prioritize, predict, discourage, and/or otherwise determine therapies (e.g., facilitate therapy determination, etc.). For example, Block S140 can include determining one or more of probiotic-based therapies, bacteriophage-based therapies, small molecule-based therapies, and/or other suitable therapies, such as therapies that can shift a subject's microbiome composition, function, diversity, and/or other characteristics (e.g., microbiomes at any suitable sites, etc.) toward a desired state (e.g., equilibrium state, etc.) in promotion of a user's health, for modifying a state of one or more appendix-related conditions, and/or for other suitable purposes.

Therapies (e.g., appendix-related therapies, etc.) can include any one or more of: consumables (e.g., probiotic therapies, prebiotic therapies, medication such as antibiotics, allergy or cold medication, bacteriophage-based therapies, consumables for underlying conditions, small molecule therapies, etc.); device-related therapies (e.g., monitoring devices; sensor-based devices; medical devices; implantable medical devices; etc.); surgical operations (e.g., appendectomies, prophylactic appendectomies, abdominal surgery, laparoscopic surgery, incision surgery; etc.); psychological-associated therapies (e.g., cognitive behavioral therapy, anxiety therapy, talking therapy, psychodynamic therapy, action-oriented therapy, rational emotive behavior therapy, interpersonal psychotherapy, relaxation training, deep breathing techniques, progressive muscle relaxation, appendix restriction therapy, meditation, etc.); behavior modification therapies (e.g., refrainment from pain remedies, antacids, laxatives, heating pads and/or other suitable treatments and/or activities; physical activity recommendations such as increased exercise; dietary recommendations such as reducing sugar intake, increased vegetable intake, increased fish intake, decreased caffeine consumption, decreased alcohol consumption, decreased carbohydrate intake; smoking recommendations such as decreasing tobacco intake; weight-related recommendations; sleep habit recommendations etc.); topical administration therapies (e.g., topical probiotic, prebiotic, and/or antibiotics; bacteriophage-based therapies); environmental factor modification therapies; modification of any other suitable aspects associated with one or more appendix-related conditions; and/or any other suitable therapies (e.g., for improving a health state associated with one or more appendix-related conditions, such as therapies for improving one or more appendix-related conditions, therapies for reducing the risk of one or more appendix-related conditions, etc.). In examples, types of therapies can include any one or more of: probiotic therapies, bacteriophage-based therapies, small molecule-based therapies, cognitive/behavioral therapies, physical rehabilitation therapies, clinical therapies, medication-based therapies, diet-related therapies, and/or any other suitable therapy designed to operate in any other suitable manner in promoting a user's health.

In variations, therapies can include site-specific therapies associated with one or more body sites, such as for facilitating modification of microbiome composition and/or function at one or more different body sites of a user (e.g., one or more different collection sites, etc.), such as targeting and/or transforming microorganisms associated with a gut site, nose site, skin site, mouth site, and/or genital site (e.g., by facilitating therapeutic intervention in relation to one or more therapies configured to specifically target one or more user body sites, such as microbiome at one or more of the user body sites; etc.), such as for facilitating improvement of one or more appendix-related conditions (e.g., by modifying user microbiome composition and/or function at a particular user body site towards a target microbiome composition and/or function, such as microbiome composition and/or function at a particular body site and associated with a healthy appendix status and/or lack of the one or more appendix-related condition; etc.). Site-specific therapies can include any one or more of consumables (e.g., targeting a gut site microbiome and/or microbiomes associated with any suitable body sites; etc.); topical therapies (e.g., for modifying a skin microbiome, a nose microbiome, a mouth microbiome, a genitals microbiome, etc.); and/or any other suitable types of therapies. In an example, the method 100 can include collecting a sample associated with a first body site (e.g., including at least one of a gut site, a skin site, a genital site, a mouth site, and a nose site, etc.) from a user; determining site-specific composition features associated with the first body site; determining an appendix-related characterization for the user for the appendix-related condition based on the site-specific composition features; and facilitating therapeutic intervention in relation to a first site-specific therapy for the user (e.g., providing the first site-specific therapy to the user; etc.) for facilitating improvement of the appendix-related condition, based on the appendix-related characterization, where the first site-specific therapy is associated with the first body site. In an example, the method 100 can include collecting a post-therapy sample from the user after the facilitation of the therapeutic intervention in relation to the first site-specific therapy (e.g., after the providing of the first site-specific therapy; etc.), where the post-therapy sample is associated with a second body site (e.g., including at least one of the gut site, the skin site, the genital site, the mouth site, and the nose site; etc.); determining a post-therapy appendix-related characterization for the user for the appendix-related condition based on site-specific features associated with the second body site; and facilitating therapeutic intervention in relation to a second site-specific therapy for the user (e.g., providing a second site-specific therapy to the user; etc.) for facilitating improvement of the appendix-related condition, based on the post-therapy appendix-related characterization, where the second site-specific therapy is associated with the second body site.

In a variation, therapies can include one or more bacteriophage-based therapies (e.g., in the form of a consumable, in the form of a topical administration therapy, etc.), where one or more populations (e.g., in terms of colony forming units) of bacteriophages specific to a certain bacteria (or other microorganism) represented in the subject can be used to down-regulate or otherwise eliminate populations of the certain bacteria. As such, bacteriophage-based therapies can be used to reduce the size(s) of the undesired population(s) of bacteria represented in the subject. Additionally or alternatively, bacteriophage-based therapies can be used to increase the relative abundances of bacterial populations not targeted by the bacteriophage(s) used. However, bacteriophage-based therapies can be used to modulate characteristics of microbiomes (e.g., microbiome composition, microbiome function, etc.) in any suitable manner, and/or can be used for any suitable purpose.

In variations, therapies can include one or more probiotic therapies and/or prebiotic therapies associated with any combination of at least one or more of (e.g., including any combination of one or more of, at any suitable amounts and/or concentrations, such as any suitable relative amounts and/or concentrations; etc.) any suitable taxa described herein (e.g., in relation to one or more microbiome composition features associated with one or more appendix-related conditions, etc.) and/or one or more of Enterococcus raffinosus, Staphylococcus sp. C912, Gemella sp. 933-88, Enterococcus sp. SI-4, Bilophila sp. 4_1_30, Anaerostipes sp. 5_1_63FAA, Phascolarctobacterium faecium, Alistipes sp. RMA 9912, Odoribacter splanchnicus, Alistipes sp. HGB5, Subdoligranulum variabile, Methanobrevibacter smithii, Lactobacillus sp. 7_1_47FAA, Flavonifractor plautii, Kluyvera georgiana, Blautia faecis, Faecalibacterium prausnitzii, Lactonifactor longoviformis, Roseburia sp. 11SE39, Bacteroides sp. AR29, Alistipes sp. NML05A004, Prevotella timonensis, Anaerostipes sp. 3_2_56FAA, Klebsiella sp. SOR89, Megasphaera sp. DNF00912, Veillonella dispar, Lactobacillus mucosae, Bacteroides fragilis, Streptococcus equinus, Bacteroides plebeius, Propionibacterium sp. MSP09A, Streptococcus pasteurianus, Anaerovibrio sp. 765, Akkermansia muciniphila, Actinomyces turicensis, Cronobacter sakazakii, Veillonella rogosae, Blautia glucerasea, Acidaminococcus intestini, Propionibacterium granulosum, Bacteroides thetaiotaomicron, Fusobacterium sp. CM21, Pediococcus sp. MFC1, Turicibacter sanguinis, Sarcina ventriculi, Megasphaera genomosp. C1, Streptococcus sp. BS35a, Streptococcus thermophilus, Fusobacterium ulcerans, Morganella morganii, Bacteroides sp. SLC1-38, Bacteroides eggerthii, Bacteroides coprocola, Bacteroides sp. CB57, Bifidobacterium stercoris, Veillonella atypica, Fusobacterium necrogenes, Lactobacillus crispatus, Veillonella sp. MSA12, Asaccharospora irregularis, Erysipelatoclostridium ramosum, Lactobacillus sp. TAB-22, Parasutterella excrementihominis, Lactobacillus sp. C412, Parabacteroides sp. 157, Bacteroides finegoldii, Alistipes putredinis, and/or any other suitable microorganisms associated with any suitable taxonomic groups (e.g., microorganisms from taxa described herein, such as in relation to microbiome features; taxa associated with functional features described herein, etc.). For one or more probiotic therapies and/or other suitable therapies, microorganisms associated with a given taxonomic group, and/or any suitable combination of microorganisms can be provided at dosages of 0.1 million to 10 billion CFU, and/or at any suitable amount (e.g., as determined from a therapy model that predicts positive adjustment of a patient's microbiome in response to the therapy; different amounts for different taxa; same or similar amounts for different taxa; etc.). In an example, a subject can be instructed to ingest capsules including the probiotic formulation according to a regimen tailored to one or more of his/her: physiology (e.g., body mass index, weight, height), demographic characteristics (e.g., gender, age), severity of dysbiosis, sensitivity to medications, and any other suitable factor. In examples, probiotic therapies and/or prebiotic therapies can be used to modulate a user microbiome (e.g., in relation to composition, function, etc.) for facilitating improvement of one or more appendix-related conditions. In examples, facilitating therapeutic intervention can include promoting (e.g., recommending, informing a user regarding, providing, administering, facilitating obtainment of, etc.) one or more probiotic therapies and/or prebiotic therapies to a user, such as for facilitating improvement of one or more appendix-related conditions.

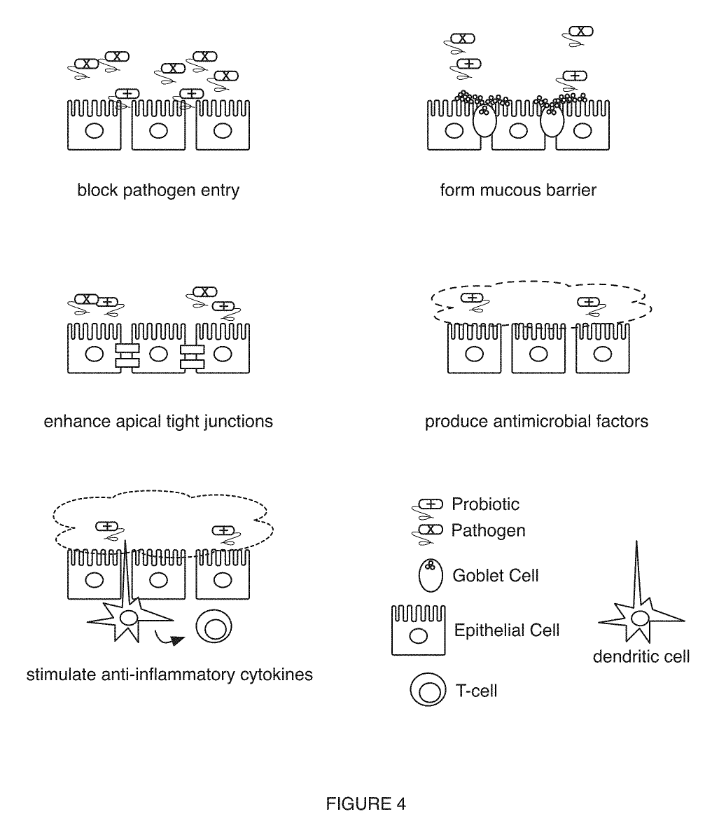

In a specific example of probiotic therapies, as shown in FIG. 4, candidate therapies of the therapy model can perform one or more of: blocking pathogen entry into an epithelial cell by providing a physical barrier (e.g., by way of colonization resistance), inducing formation of a mucous barrier by stimulation of goblet cells, enhance integrity of apical tight junctions between epithelial cells of a subject (e.g., by stimulating up regulation of zona-occludens 1, by preventing tight junction protein redistribution), producing antimicrobial factors, stimulating production of anti-inflammatory cytokines (e.g., by signaling of dendritic cells and induction of regulatory T-cells), triggering an immune response, and performing any other suitable function that adjusts a subject's microbiome away from a state of dysbiosis. However, probiotic therapies and/or prebiotic therapies can be configured in any suitable manner.

In another specific example, therapies can include medical-device based therapies (e.g., associated with human behavior modification, associated with treatment of disease-related conditions, etc.).

In variations, the therapy model is preferably based upon data from a large population of subjects, which can include the population of subjects from which the microbiome diversity datasets are derived in Block Silo, where microbiome composition and/or functional features or states of health, prior exposure to and post exposure to a variety of therapeutic measures, are well characterized. Such data can be used to train and validate the therapy provision model, in identifying therapeutic measures that provide desired outcomes for subjects based upon different appendix-related characterizations. In variations, support vector machines, as a supervised machine learning algorithm, can be used to generate the therapy provision model. However, any other suitable machine learning algorithm described above can facilitate generation of the therapy provision model.

Additionally or alternatively, the therapy model can be derived in relation to identification of a “normal” or baseline microbiome composition and/or functional features, as assessed from subjects of a population of subjects who are identified to be in good health. Upon identification of a subset of subjects of the population of subjects who are characterized to be in good health (e.g., using features of the characterization process), therapies that modulate microbiome compositions and/or functional features toward those of subjects in good health can be generated in Block S140. Block S140 can thus include identification of one or more baseline microbiome compositions and/or functional features (e.g., one baseline microbiome for each of a set of demographic characteristics), and potential therapy formulations and therapy regimens that can shift microbiomes of subjects who are in a state of dysbiosis toward one of the identified baseline microbiome compositions and/or functional features. The therapy model can, however, be generated and/or refined in any other suitable manner.

Microorganism compositions associated with probiotic therapies and/or prebiotic therapies (e.g., associated with probiotic therapies determined by a therapy model applied by a therapy facilitation system, etc.) can include microorganisms that are culturable (e.g., able to be expanded to provide a scalable therapy) and/or non-lethal (e.g., non-lethal in their desired therapeutic dosages). Furthermore, microorganism compositions can include a single type of microorganism that has an acute or moderated effect upon a subject's microbiome. Additionally or alternatively, microorganism compositions can include balanced combinations of multiple types of microorganisms that are configured to cooperate with each other in driving a subject's microbiome toward a desired state. For instance, a combination of multiple types of bacteria in a probiotic therapy can include a first bacteria type that generates products that are used by a second bacteria type that has a strong effect in positively affecting a subject's microbiome. Additionally or alternatively, a combination of multiple types of bacteria in a probiotic therapy can include several bacteria types that produce proteins with the same functions that positively affect a subject's microbiome.

Probiotic and/or prebiotic compositions can be naturally or synthetically derived. For instance, in one application, a probiotic composition can be naturally derived from fecal matter or other biological matter (e.g., of one or more subjects having a baseline microbiome composition and/or functional features, as identified using the characterization process and the therapy model). Additionally or alternatively, probiotic compositions can be synthetically derived (e.g., derived using a benchtop method) based upon a baseline microbiome composition and/or functional features, as identified using the characterization process and the therapy model. In variations, microorganism agents that can be used in probiotic therapies can include one or more of: yeast (e.g., Saccharomyces boulardii), gram-negative bacteria (e.g., E. coli Nissle), gram-positive bacteria (e.g., Bifidobacteria bifidum, Bifidobacteria infantis, Lactobacillus rhamnosus, Lactococcus lactis, Lactobacillus plantarum, Lactobacillus acidophilus, Lactobacillus casei, Bacillus polyfermenticus, etc.), and any other suitable type of microorganism agent. However, probiotic therapies, prebiotic therapies and/or other suitable therapies can include any suitable combination of microorganisms associated with any suitable taxa described herein, and/or therapies can be configured in any suitable manner.

Block S140 can include executing, storing, retrieving, and/or otherwise processing one or more therapy models for determining one or more therapies. Processing one or more therapy models is preferably based on microbiome features. For example, generating a therapy model can based on microbiome features associated with one or more appendix-related conditions, therapy-related aspects such as therapy efficacy in relation to microbiome characteristics, and/or other suitable data. Additionally or alternatively, processing therapy models can be based on any suitable data. In an example, processing a therapy model can include determining one or more therapies for a user based on one or more therapy models, user microbiome features (e.g., inputting user microbiome feature values into the one or more therapy models, etc.), supplementary data (e.g., prior knowledge associated with therapies such as in relation to microorganism-related metabolization; user medical history; user demographic data, such as describing demographic characteristics; etc.), and/or any other suitable data. However, processing therapy models can be based on any suitable data in any suitable manner.

Appendix-related characterization models can include one or more therapy models. In an example, determining one or more appendix-related characterizations (e.g., for one or more users, for one or more appendix-related conditions, etc.), can include determining one or more therapies, such as based on one or more therapy models (e.g., applying one or more therapy models, etc.) and/or other suitable data (e.g., microbiome features such as user microbiome features, microorganism dataset such as user microorganism datasets, etc.). In a specific example, determining one or more appendix-related characterizations can include determining a first appendix-related characterization for a user (e.g., describing propensity for one or more appendix-related conditions; etc.); and determining a second appendix-related characterization for the user based on the first appendix-related characterization (e.g., determining one or more therapies, such as for recommendation to a user, based on the propensity for one or more appendix-related conditions; etc.). In a specific example, an appendix-related characterization can include both propensity-related data (e.g., diagnostic data; associated microbiome composition, function, diversity, and/or other characteristics; etc.) and therapy-related data (e.g., recommended therapies; potential therapies; etc.). However, appendix-related characterizations can include any suitable data (e.g., any combination of data described herein, etc.).

Processing therapy models can include processing a plurality of therapy models. For example, different therapy models can be processed for different therapies (e.g., different models for different individual therapies; different models for different combinations and/or categories of therapies, such as a first therapy model for determining consumable therapies and a second therapy model for determining psychological-associated therapies; etc.). In an example, different therapy models can be processed for different appendix-related conditions, (e.g., different models for different individual appendix-related conditions; different models for different combinations and/or categories of appendix-related conditions, etc.). Additionally or alternatively, processing a plurality of therapy models can be performed for (e.g., based on; processing different therapy models for; etc.) any suitable types of data and/or entities. However, processing a plurality of therapy models can be performed in any suitable manner, and determining and/or applying one or more therapy models can be performed in any suitable manner.

### 4.4 Processing a User Biological Sample.

Embodiments of the method 100 can additionally or alternatively include Block S150, which can include processing one or more biological samples from a user (e.g., biological samples from different collection sites of the user, etc.). Block S150 can function to facilitate generation of a microorganism dataset for a user, such as for use in deriving inputs for the characterization process (e.g., for generating an appendix-related characterization for the user, such as through applying one or more appendix-related characterization models, etc.). As such, Block S150 can include receiving, processing, and/or analyzing one or more biological samples from one or more users (e.g., multiple biological samples for the same user over time, different biological samples for different users, etc.). In Block S150, the biological sample is preferably generated from the user and/or an environment of the user in a non-invasive manner. In variations, non-invasive manners of sample reception can use any one or more of: a permeable substrate (e.g., a swab configured to wipe a region of a user's body, toilet paper, a sponge, etc.), a non-permeable substrate (e.g., a slide, tape, etc.) a container (e.g., vial, tube, bag, etc.) configured to receive a sample from a region of a user's body, and any other suitable sample-reception element. In a specific example, the biological sample can be collected from one or more of the user's nose, skin, genitals, mouth, and gut (e.g., through stool samples, etc.) in a non-invasive manner (e.g., using a swab and a vial). However, the biological sample can additionally or alternatively be received in a semi-invasive manner or an invasive manner. In variations, invasive manners of sample reception can use any one or more of: a needle, a syringe, a biopsy element, a lance, and any other suitable instrument for collection of a sample in a semi-invasive or invasive manner. In specific examples, samples can include blood samples, plasma/serum samples (e.g., to enable extraction of cell-free DNA), and tissue samples.

In the above variations and examples, the biological sample can be taken from the body of the user without facilitation by another entity (e.g., a caretaker associated with a user, a health care professional, an automated or semi-automated sample collection apparatus, etc.), or can alternatively be taken from the body of the user with the assistance of another entity. In one example, where the biological sample is taken from the user without facilitation by another entity in the sample extraction process, a sample-provision kit can be provided to the user. In the example, the kit can include one or more swabs for sample acquisition, one or more containers configured to receive the swab(s) for storage, instructions for sample provision and setup of a user account, elements configured to associate the sample(s) with the user (e.g., barcode identifiers, tags, etc.), and a receptacle that allows the sample(s) from the user to be delivered to a sample processing operation (e.g., by a mail delivery system). In another example, where the biological sample is extracted from the user with the help of another entity, one or more samples can be collected in a clinical or research setting from the user (e.g., during a clinical appointment). The biological sample can, however, be received from the user in any other suitable manner.

Furthermore, processing and analyzing biological samples (e.g., to generate a user microorganism dataset; etc.) from the user is preferably performed in a manner similar to that of one of the embodiments, variations, and/or examples of sample reception described in relation to Block Silo above, and/or any other suitable portions of embodiments of the method 100 and/or system 200. As such, reception and processing of the biological sample in Block S150 can be performed for the user using similar processes as those for receiving and processing biological samples used to perform the characterization processes of the method 100, such as in order to provide consistency of process. However, biological sample reception and processing in Block S150 can additionally or alternatively be performed in any other suitable manner.

### 4.5 Determining an Appendix-Related Characterization.

Embodiments of the method 100 can additionally or alternatively include Block S160, which can include determining, with one or more characterization processes (e.g., one or more characterization processes described in relation to Block S130, etc.), an appendix-related characterization for the user, such as based upon processing one or more microorganism dataset (e.g., user microorganism sequence dataset, microbiome composition dataset, microbiome functional diversity dataset; processing of the microorganism dataset to extract user microbiome features (e.g., extract feature values; etc.) that can be used to determine the one or more appendix-related characterizations; etc.) derived from the biological sample of the user. Block S160 can function to characterize one or more appendix-related conditions for a user, such as through extracting features from microbiome-derived data of the user, and using the features as inputs into an embodiment, variation, or example of the characterization process described in Block S130 above (e.g., using the user microbiome feature values as inputs into a microbiome-related condition characterization model, etc.). In an example, Block S160 can include generating an appendix-related characterization for the user based on user microbiome features and an appendix-related condition model (e.g., generated in Block S130). Appendix-related characterizations can be for any number and/or combination of appendix-related conditions (e.g., a combination of appendix-related conditions, a single appendix-related condition, and/or other suitable appendix-related conditions; etc.), users, collection sites, and/or other suitable entities. Appendix-related characterizations can include one or more of: diagnoses (e.g., presence or absence of an appendix-related condition; etc.); risk (e.g., risk scores for developing and/or the presence of an appendix-related condition; information regarding appendix-related characterizations (e.g., symptoms, signs, triggers, associated conditions, etc.); comparisons (e.g., comparisons with other subgroups, populations, users, historic health statuses of the user such as historic microbiome compositions and/or functional diversities; comparisons associated with appendix-related conditions; etc.); therapy determinations; other suitable outputs associated with characterization processes; and/or any other suitable data.

In another variation, an appendix-related characterization can include a microbiome diversity score (e.g., in relation to microbiome composition, function, etc.) associated with (e.g., correlated with; negatively correlated with; positively correlated with; etc.) a microbiome diversity score correlated with one or more appendix-related conditions. In examples, the appendix-related characterization can include microbiome diversity scores over time (e.g., calculated for a plurality of biological samples of the user collected over time), comparisons to microbiome diversity scores for other users, and/or any other suitable type of microbiome diversity score. However, processing microbiome diversity scores (e.g., determining microbiome diversity scores; using microbiome diversity scores to determine and/or provide therapies; etc.) can be performed in any suitable manner.

Determining an appendix-related characterization in Block S160 preferably includes determining features and/or combinations of features associated with the microbiome composition and/or functional features of the user (e.g., determining feature values associated with the user, the feature values corresponding to microbiome features determined in Block S130, etc.), inputting the features into the characterization process, and receiving an output that characterizes the user as belonging to one or more of: a behavioral group, a gender group, a dietary group, a disease-state group, and any other suitable group capable of being identified by the characterization process. Block S160 can additionally or alternatively include generation of and/or output of a confidence metric associated with the characterization of the user. For instance, a confidence metric can be derived from the number of features used to generate the characterization, relative weights or rankings of features used to generate the characterization, measures of bias in the characterization process, and/or any other suitable parameter associated with aspects of the characterization process. However, leveraging user microbiome features can be performed in any suitable manner to generate any suitable appendix-related characterizations.

In some variations, features extracted from the microorganism dataset of the user can be supplemented with supplementary features (e.g., extracted from supplementary data collected for the user; such as survey-derived features, medical history-derived features, sensor data, etc.), where such data, the user microbiome data, and/or other suitable data can be used to further refine the characterization process of Block S130, Block S160, and/or other suitable portions of embodiments of the method 100.

Determining an appendix-related characterization preferably includes extracting and applying user microbiome features (e.g., user microbiome composition diversity features; user microbiome functional diversity features; extracting feature values; etc.) for the user (e.g., based on a user microorganism dataset), characterization models, and/or other suitable components, such as by employing processes described in Block S130, and/or by employing any suitable approaches described herein.

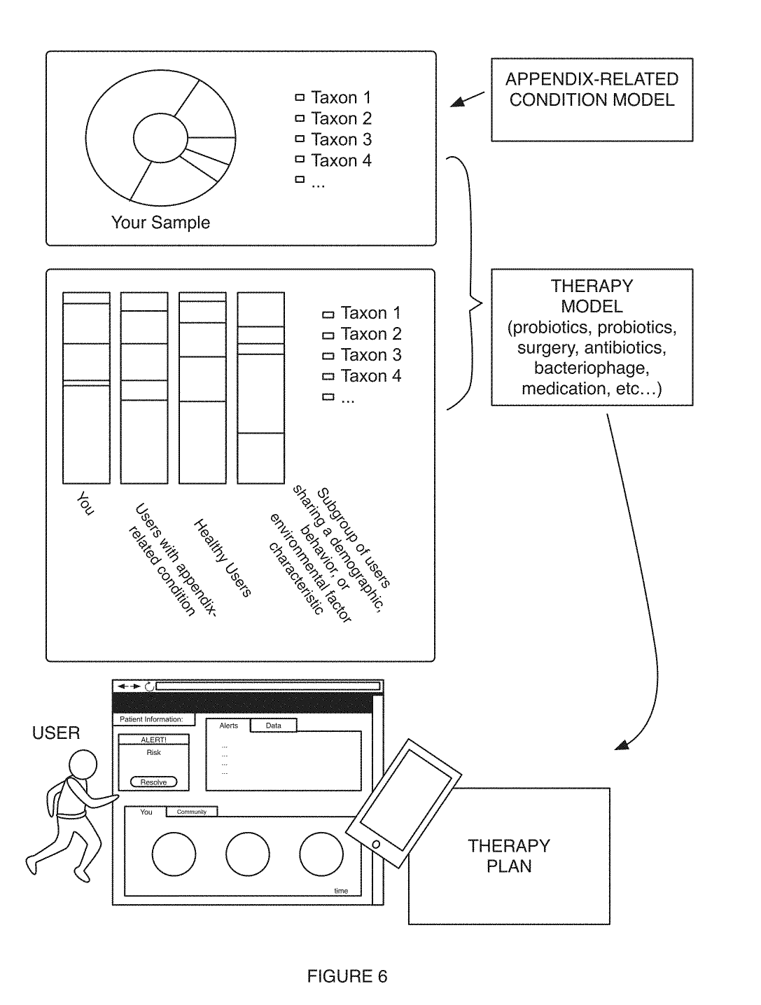

In variations, as shown in FIG. 6, Block S160 can include presenting appendix-related characterizations (e.g., information extracted from the characterizations; as part of facilitating therapeutic intervention; etc.), such as at a web interface, a mobile application, and/or any other suitable interface, but presentation of information can be performed in any suitable manner. However, the microorganism dataset of the user can additionally or alternatively be used in any other suitable manner to enhance the models of the method 100, and Block S160 can be performed in any suitable manner.

### 4.6 Facilitating Therapeutic Intervention.

As shown in FIG. 9, embodiments of the method 100 can additionally or alternatively include Block S170, which can include facilitating therapeutic intervention (e.g., promoting therapies, providing therapies, facilitating provision of therapies, etc.) for one or more appendix-related conditions for one or more users (e.g., based upon an appendix-related characterization and/or a therapy model). Block S170 can function to recommend, promote, provide, and/or otherwise facilitate therapeutic intervention in relation to one or more therapies for a user, such as to shift the microbiome composition and/or functional diversity of a user toward a desired equilibrium state (and/or otherwise improving a state of the appendix-related condition, etc.) in relation to one or more appendix-related conditions. Block S170 can include provision of a customized therapy to the user according to their microbiome composition and functional features, where the customized therapy can include a formulation of microorganisms configured to correct dysbiosis characteristic of users having the identified characterization. As such, outputs of Block S140 can be used to directly promote a customized therapy formulation and regimen (e.g., dosage, usage instructions) to the user based upon a trained therapy model. Additionally or alternatively, therapy provision can include recommendation of available therapeutic measures configured to shift microbiome composition and/or functional features toward a desired state. In variations, therapies can include any one or more of: consumables, topical therapies (e.g., lotions, ointments, antiseptics, etc.), medication (e.g., medications associated with any suitable medication type and/or dosage, etc.), bacteriophages, environmental treatments, behavioral modification (e.g., diet modification therapies, stress-reduction therapies, physical activity-related therapies, etc.), diagnostic procedures, other medical-related procedures, and/or any other suitable therapies associated with appendix-related conditions. Consumables can include any one or more of: food and/or beverage items (e.g., probiotic and/or prebiotic food and/or beverage items, etc.), nutritional supplements (e.g., vitamins, minerals, fiber, fatty acids, amino acids, prebiotics, probiotics, etc.), consumable medications, and/or any other suitable therapeutic measure. In an example, providing one or more therapies and/or otherwise facilitating therapeutic intervention can include providing a recommendation for the one or more therapies to one or more users at one or more computing devices (e.g., at a user interface such as a web application, presented at the computing devices; etc.) associated with the one or more users.

For example, a combination of commercially available probiotic supplements can include a suitable probiotic therapy for the user according to an output of the therapy model. In another example, the method 100 can include determining an appendix-related condition risk for the user for the appendix-related condition based on an appendix-related condition model (e.g., and/or user microbiome features); and promoting a therapy to the user based on the appendix-related condition risk.

In a variation, facilitating therapeutic intervention can include promoting a diagnostic procedure (e.g., for facilitating detection of appendix-related conditions, which can motivate subsequent promotion of other therapies, such as for modulation of a user microbiome for improving a user health state associated with one or more appendix-related conditions; etc.). Diagnostic procedures can include any one or more of: medical history analyses, imaging examinations, cell culture tests, antibody tests, skin prick testing, patch testing, blood testing, challenge testing, performing portions of embodiments of the method 100, and/or any other suitable procedures for facilitating the detecting (e.g., observing, predicting, etc.) of appendix-related conditions. Additionally or alternatively, diagnostic device-related information and/or other suitable diagnostic information can be processed as part of a supplementary dataset (e.g., in relation to Block S120, where such data can be used in determining and/or applying characterization models, therapy models, and/or other suitable models; etc.), and/or collected, used, and/or otherwise processed in relation to any suitable portions of embodiments of the method 100 (e.g., administering diagnostic procedures for users for monitoring therapy efficacy in relation to Block S180; etc.)

In another variation, Block S170 can include promoting a bacteriophage-based therapy. In more detail, one or more populations (e.g., in terms of colony forming units) of bacteriophages specific to a certain bacteria (or other microorganism) represented in the user can be used to down-regulate or otherwise eliminate populations of the certain bacteria. As such, bacteriophage-based therapies can be used to reduce the size(s) of the undesired population(s) of bacteria represented in the user. Complementarily, bacteriophage-based therapies can be used to increase the relative abundances of bacterial populations not targeted by the bacteriophage(s) used.

In another variation, facilitating therapeutic intervention (e.g., providing therapies, etc.) can include provision of notifications to a user regarding the recommended therapy, other forms of therapy, appendix-related characterizations, and/or other suitable data. In a specific example, providing a therapy to a user can include providing therapy recommendations (e.g., substantially concurrently with providing information derived from an appendix-related characterization for a user; etc.) and/or other suitable therapy-related information (e.g., therapy efficacy; comparisons to other individual users, subgroups of users, and/or populations of users; therapy comparisons; historic therapies and/or associated therapy-related information; psychological therapy guides such as for cognitive behavioral therapy; etc.), such as through presenting notifications at a web interface (e.g., through a user account associated with and identifying a user; etc.). Notifications can be provided to a user by way of an electronic device (e.g., personal computer, mobile device, tablet, wearable, head-mounted wearable computing device, wrist-mounted wearable computing device, etc.) that executes an application, web interface, and/or messaging client configured for notification provision. In one example, a web interface of a personal computer or laptop associated with a user can provide access, by the user, to a user account of the user, where the user account includes information regarding the user's appendix-related characterization, detailed characterization of aspects of the user's microbiome (e.g., in relation to correlations with appendix-related conditions; etc.), and/or notifications regarding suggested therapeutic measures (e.g., generated in Blocks S140 and/or S170, etc.). In another example, an application executing at a personal electronic device (e.g., smart phone, smart watch, head-mounted smart device) can be configured to provide notifications (e.g., at a display, haptically, in an auditory manner, etc.) regarding therapy suggestions generated by the therapy model of Block S170. Notifications and/or probiotic therapies can additionally or alternatively be provided directly through an entity associated with a user (e.g., a caretaker, a spouse, a significant other, a healthcare professional, etc.). In some further variations, notifications can additionally or alternatively be provided to an entity (e.g., healthcare professional) associated with a user, such as where the entity is able to facilitate provision of the therapy (e.g., by way of prescription, by way of conducting a therapeutic session, through a digital telemedicine session using optical and/or audio sensors of a computing device, etc.). Providing notifications and/or otherwise facilitating therapeutic, however, be performed in any suitable manner.

### 4.7 Monitoring Therapy Effectiveness.

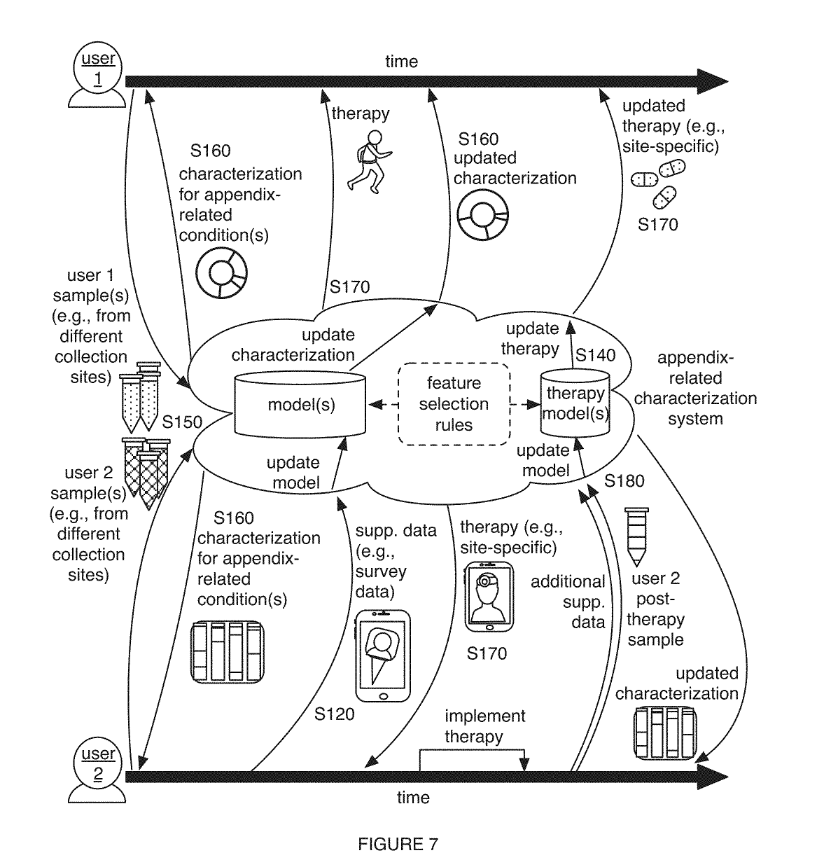

As shown in FIG. 7, the method can additionally or alternatively include Block S180, which can include: monitoring effectiveness of one or more therapies and/or monitoring other suitable components (e.g., microbiome characteristics, etc.) for the user (e.g., based upon processing a series of biological samples from the user), over time. Block S180 can function to gather additional data regarding positive effects, negative effects, and/or lack of effectiveness of one or more therapies (e.g., suggested by the therapy model for users of a given characterization, etc.) and/or monitoring microbiome characteristics (e.g., to assess microbiome composition and/or functional features for the user at a set of time points, etc.).

Monitoring of a user during the course of a therapy promoted by the therapy model (e.g., by receiving and analyzing biological samples from the user throughout therapy, by receiving survey-derived data from the user throughout therapy) can thus be used to generate a therapy-effectiveness model for each characterization provided by the characterization process of Block S130, and each recommended therapy measure provided in Blocks S140 and S170.

In Block S180, the user can be prompted to provide additional biological samples, supplementary data, and/or other suitable data at one or more key time points of a therapy regimen that incorporates the therapy, and the additional biological sample(s) can be processed and analyzed (e.g., in a manner similar to that described in relation to Block S120) to generate metrics characterizing modulation of the user's microbiome composition and/or functional features. For instance, metrics related to one or more of: a change in relative abundance of one or more taxonomic groups represented in the user's microbiome at an earlier time point, a change in representation of a specific taxonomic group of the user's microbiome, a ratio between abundance of a first taxonomic group of bacteria and abundance of a second taxonomic group of bacteria of the user's microbiome, a change in relative abundance of one or more functional families in a user's microbiome, and any other suitable metrics can be used to assess therapy effectiveness from changes in microbiome composition and/or functional features. Additionally or alternatively, survey-derived data from the user, pertaining to experiences of the user while on the therapy (e.g., experienced side effects, personal assessment of improvement, behavioral modifications, symptom improvement, etc.) can be used to determine effectiveness of the therapy in Block S180. For example, the method 100 can include receiving a post-therapy biological sample from the user; collecting a supplementary dataset from the user, where the supplementary dataset describes user adherence to a therapy (e.g., a determined and promoted therapy) and/or other suitable user characteristics (e.g., behaviors, conditions, etc.); generating a post-therapy appendix-related characterization of the first user in relation to the appendix-related condition based on the appendix-related characterization model and the post-therapy biological sample; and promoting an updated therapy to the user for the appendix-related condition based on the post-therapy appendix-related characterization (e.g., based on a comparison between the post-therapy appendix-related characterization and a pre-therapy appendix-related characterization; etc.) and/or the user adherence to the therapy (e.g., modifying the therapy based on positive or negative results for the user microbiome in relation to the appendix-related condition; etc.). Additionally or alternatively, other suitable data (e.g., supplementary data describing user behavior associated with one or more appendix-related conditions; supplementary data describing an appendix-related condition such as observed symptoms; etc.) can be used in determining a post-therapy characterization (e.g., degree of change from pre- to post-therapy in relation to the appendix-related condition; etc.), updated therapies (e.g., determining an updated therapy based on effectiveness and/or adherence to the promoted therapy, etc.).

In an example, the method 100 can include collecting supplementary data (e.g., survey-derived data; informing statuses of appendix-related conditions, such as in relation to symptom severity; etc.); determining the appendix-related characterization for the user based on the user microbiome features and the supplementary data; facilitating therapeutic intervention in relation to a therapy for the appendix-related condition (e.g., promoting the therapy to the user; etc.), based on the appendix-related characterization; collecting a post-therapy biological sample from the user (e.g., after facilitating the therapeutic intervention; etc.); collecting subsequent supplementary data (e.g., including at least one of second survey-derived data and device data; etc.); and determining a post-therapy appendix-related characterization for the user for the appendix-related condition based on the subsequent supplementary data and post-therapy user microbiome features associated with the post-therapy biological sample. In the example, the method 100 can include facilitating therapeutic intervention in relation to an updated therapy (e.g., a modification of the therapy; a different therapy; etc.) for the user for improving the appendix-related condition, based on the post-therapy appendix-related characterization, such as where the updated therapy can include at least one of a consumable, a device-related therapy, a surgical operation, a psychological-associated therapy, a behavior modification therapy, and an environmental factor modification therapy. In the example determining the post-therapy appendix-related characterization can include determining a comparison between microbiome characteristics of the user and reference microbiome characteristics corresponding to a user subgroup sharing at least one of a behavior and an environmental factor (and/or other suitable characteristic) associated with the appendix-related condition, based on the post-therapy microbiome features, and where facilitating therapeutic intervention in relation to the updated therapy can include presenting the comparison to the user for facilitating at least one of the behavior modification therapy and the environmental factor modification therapy and/or other suitable therapies. However, Block S180 can be performed in relation to additional biological samples, additional supplementary data, and/or other suitable additional data in any suitable manner.

Therapy effectiveness, processing of additional biological samples (e.g., to determine additional appendix-related characterizations, therapies, etc.), and/or other suitable aspects associated with continued biological sample collection, processing, and analysis in relation to appendix-related conditions can be performed at any suitable time and frequency for generating, updating, and/or otherwise processing models (e.g., characterization models, therapy models, etc.), and/or for any other suitable purpose (e.g., as inputs associated with other portions of embodiments of the method 100). However, Block S180 can be performed in any suitable manner.

Embodiments of the method 100 can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

Embodiments of the method 100 and/or system 200 can include every combination and permutation of the various system components and the various method processes, including any variants (e.g., embodiments, variations, examples, specific examples, figures, etc.), where portions of embodiments of the method 100 and/or processes described herein can be performed asynchronously (e.g., sequentially), concurrently (e.g., in parallel), or in any other suitable order by and/or using one or more instances, elements, components of, and/or other aspects of the system 200 and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method 100 and/or system 200 can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method 100, system 200, and/or variants without departing from the scope defined in the claims.

We claim:

1. A method for characterizing an appendix-related condition associated with microorganisms, the method comprising:
determining a microorganism sequence dataset associated with a set of subjects, based on microorganism nucleic acids from samples associated with the set of subjects, wherein the samples comprise at least one sample associated with the appendix-related condition;
collecting, for the set of subjects, supplementary data associated with the appendix-related condition;
determining a set of microbiome features comprising at least one of a set of microbiome composition features and a set of microbiome functional features, based on the microorganism sequence dataset;
generating an appendix-related characterization model based on the supplementary data and the set of microbiome features, wherein the appendix-related characterization model is associated with the appendix-related condition;
determining an appendix-related characterization for a user for the appendix-related condition based on the appendix-related characterization model; and
providing a therapy to the user for facilitating improvement of the appendix-related condition, based on the appendix-related characterization.

2. The method of claim 1, wherein the samples comprise first site-specific samples associated with a gut site, wherein determining the set of microbiome features comprises determining, based on the microorganism sequence dataset, the set of microbiome composition features comprising first site-specific composition features associated with the gut site and at least one of Neisseriaceae (family), Neisseria mucosa (species), Aggregatibacter aphrophilus (species), Bacteroides uniformis (species), Bacteroides vulgatus (species), Parabacteroides distasonis (species), Megasphaera (genus), Proteobacteria (phylum), Micrococcaceae (family), Streptococcus thermophilus (species), Streptococcus parasanguinis (species), Gemella (genus), Clostridium (genus), Actinomyces odontolyticus (species), Actinomycetales (order), Actinomycetaceae (family), Betaproteobacteria (class), Gemella morbillorum (species), Rothia (genus), Lactobacillus crispatus (species), Pseudomonadales (order), Oxalobacteraceae (family), Burkholderiales (order), Gemella sp. 933-88 (species), Micrococcales (order), Bacteroides acidifaciens (species), Mogibacterium (genus), Bacteroides sp. AR20 (species), Bacteroides sp. AR29 (species), Burkholderiaceae (family), Erysipelotrichaceae (family), Xanthomonadales (order), Pseudomonadaceae (family), Actinomyces sp. oral strain Hal-1065 (species), Roseburia intestinalis (species), Porphyromonadaceae (family), Shuttleworthia (genus), Clostridia (class), Clostridiales (order), Peptostreptococcaceae (family), Peptococcaceae (family), Carnobacteriaceae (family), Dialister sp. E2_20 (species), Neisseriales (order), Megasphaera genomosp. C1 (species), Moryella (genus), Synergistetes (phylum), Erysipelotrichia (class), Erysipelotrichales (order), Clostridiales Family XIII. Incertae Sedis (family), Roseburia sp. 11SE39 (species), Bacteroides sp. D22 (species), Synergistia (class), Synergistales (order), Synergistaceae (family), Lactobacillus sp. TAB-22 (species), Flavonifractor (genus), Sutterellaceae (family), Anaerostipes sp. 5_1_63FAA (species), Streptococcus sp. 2011_Oral_MS_A3 (species), Veillonella sp. 2011_Oral_VSA_D3 (species), Finegoldia sp. S9 AA1-5 (species), Fretibacterium (genus), Staphylococcus sp. 334802 (species), Peptoclostridium (genus), Intestinibacter (genus), Acinetobacter (genus), Klebsiella (genus), Bacteroides thetaiotaomicron (species), Butyrivibrio (genus), Fusobacterium necrogenes (species), Herbaspirillum (genus), Herbaspirillum seropedicae (species), Pediococcus (genus), Finegoldia magna (species), Blautia hansenii (species), Enterococcus faecalis (species), Lactococcus lactis (species), Bacillus (genus), Clostridioides difficile (species), Blautia coccoides (species), Erysipelatoclostridium ramosum (species), Weissella confusa (species), Lactobacillus plantarum (species), Lactobacillus paracasei (species), Bifidobacterium adolescentis (species), Bifidobacterium breve (species), Bifidobacterium dentium (species), Bifidobacterium animalis (species), Bifidobacterium pseudocatenulatum (species), Bacteroides ovatus (species), Peptoniphilus lacrimalis (species), Anaerococcus vaginalis (species), Rahnella (genus), Bilophila wadsworthia (species), Sneathia sanguinegens (species), Succiniclasticum (genus), Sporobacter (genus), Pseudobutyrivibrio ruminis (species), Weissella (genus), Bacteroides stercoris (species), Lactobacillus rhamnosus (species), Pantoea (genus), Holdemania (genus), Holdemania filiformis (species), Thermoanaerobacterales (order), Bifidobacterium gallicum (species), Bifidobacterium pullorum (species), Leuconostocaceae (family), Eggerthella lenta (species), Papillibacter (genus), Anaerostipes caccae (species), Pseudoflavonifractor capillosus (species), Anaerovorax (genus), Parasporobacterium (genus), Parasporobacterium paucivorans (species), Oscillospira (genus), Oscillospira guilliermondii (species), Actinomyces turicensis (species), Anaerosinus (genus), Sneathia (genus), Brevibacterium paucivorans (species), Lactobacillus sp. CR-609S (species), Thermoanaerobacteraceae (family), Bacillaceae (family), Gelria (genus), Acidobacteriales (order), Bacteroides massiliensis (species), Rhodocyclales (order), Anaerofustis stercorihominis (species), Alistipes finegoldii (species), Oscillospiraceae (family), Peptoniphilus sp. 2002-38328 (species), Hespellia (genus), Bacteroides sp. 35AE37 (species), Marvinbryantia (genus), Anaerosporobacter mobilis (species), Anaerofustis (genus), Catabacter (genus), Flavonifractor plautii (species), Proteiniphilum (genus), Roseburia faecis (species), Streptococcus sp. S16-11 (species), Bacteroides sp. 4072 (species), Alistipes shahii (species), Bacteroides intestinalis (species), Lactonifactor longoviformis (species), Bifidobacterium tsurumiense (species), Bacteroides dorei (species), Bacteroides xylanisolvens (species), Cronobacter (genus), Alloscardovia (genus), Alloscardovia omnicolens (species), Lactonifactor (genus), Catabacteriaceae (family), Adlercreutzia equolifaciens (species), Adlercreutzia (genus), Alistipes sp. EBA6-25c12 (species), Bacteroides sp. EBA5-17 (species), Oscillibacter (genus), Gordonibacter pamelaeae (species), Alistipes sp. NML05A004 (species), Parasutterella excrementihominis (species), Mitsuokella sp. DJF_RR21 (species), Butyricimonas (genus), Bifidobacterium stercoris (species), Alistipes indistinctus (species), Gordonibacter (genus), Anaerostipes hadrus (species), Klebsiella sp. B12 (species), Alistipes sp. RMA 9912 (species), Anaerosporobacter (genus), Bacteroides faecis (species), Blautia sp. Ser5 (species), Bacteroides chinchillae (species), Bilophila sp. 4_1_30 (species), Caldicoprobacteraceae (family), Enterobacter sp. UDC345 (species), Bifidobacterium biavatii (species), Peptoniphilus sp. 1-14 (species), Alistipes sp. HGB5 (species), Bacteroides sp. SLC1-38 (species), Lactobacillus sp. Akhmrol (species), Klebsiella sp. SOR89 (species), Enterococcus sp. C6 I11 (species), Pseudoflavonifractor (genus), Bacteroides sp. dnLKV9 (species), Megasphaera sp. BV3C16-1 (species), Faecalibacterium sp. canine oral taxon 147 (species), Varibaculum sp. CCUG 45114 (species), Butyricimonas sp. 214-4 (species), Anaerostipes rhamnosivorans (species), Negativicoccus sp. S5-A15 (species), [Collinsella] massiliensis (species), Corynebacterium sp. jw37 (species), Roseburia sp. 499 (species), Dialister sp. S7MSR5 (species), Anaerococcus sp. S8 87-3 (species), Finegoldia sp. S8 F7 (species), Murdochiella sp. S9 PR-10 (species), Peptoniphilus sp. S9 PR-13 (species), Bacteroides sp. J1511 (species), Corynebacterium sp. 713182/2012 (species), Rahnella sp. BSP18 (species), Intestinimonas (genus), Robinsoniella sp. KNHs210 (species), Candidatus Soleaferrea (genus), Butyricimonas faecihominis (species), Senegalimassilia (genus), Peptoniphilus sp. DNF00840 (species), Romboutsia (genus), and Coprobacter secundus (species), wherein generating the appendix-related characterization model comprises generating a first site-specific appendix-related characterization model based on the supplementary data and the first site-specific composition features, and wherein determining the appendix-related characterization comprises determining the appendix-related characterization for the user for the appendix-related condition based on the first site-specific appendix-related characterization model.

3. The method of claim 2, wherein determining the set of microbiome features comprises determining, based on the microorganism sequence dataset, the set of microbiome functional features comprising site-specific functional features associated with the gut site and at least one of Neurodegenerative Disease, Signaling Molecules and Interaction, Xenobiotics Biodegradation and Metabolism, Ascorbate and aldarate metabolism, Huntington's disease, Inositol phosphate metabolism, Propanoate metabolism, Starch and sucrose metabolism, Caprolactam degradation, Cell motility and secretion, Valine, leucine and isoleucine degradation, Tryptophan metabolism, Type I diabetes mellitus, Phenylalanine metabolism, Selenocompound metabolism, Lysine degradation, Polycyclic aromatic hydrocarbon degradation, Glycan biosynthesis and metabolism, Renal cell carcinoma, Butanoate metabolism, Carbon fixation pathways in prokaryotes, Citrate cycle (TCA cycle), Lipopolysaccharide biosynthesis, RNA transport, Thiamine metabolism, 1,1,1-Trichloro-2,2-bis (4-chlorophenyl)ethane (DDT) degradation, Electron transfer carriers, Amyotrophic lateral sclerosis (ALS), Prion disease, Toluene degradation, and alpha-Linolenic acid metabolism, wherein generating the appendix-related characterization model comprises generating the first site-specific appendix-related characterization model based on the supplementary data, the first site-specific composition features, and the site-specific functional features.

4. The method of claim 2, further comprising:
collecting second site-specific samples associated with at least one of a skin site, a genital site, a mouth site, and a nose site;
determining second site-specific composition features associated with the at least one of the skin site, the genital site, the mouth site, and the nose site, wherein the second site-specific composition features are associated with at least one of Gemella (genus), Veillonella atypica (species), Dialister pneumosintes (species), Lactobacillus crispatus (species), Phyllobacteriaceae (family), Aquabacterium (genus), Anaeroglobus (genus), Anaeroglobus geminatus (species), Ochrobactrum (genus), Mobiluncus curtisii (species), Actinomyces neuii (species), Anaerococcus lactolyticus (species), Lactobacillus johnsonii (species), Verrucomicrobiales (order), Verrucomicrobia (phylum), Verrucomicrobiae (class), Verrucomicrobiaceae (family), Dialister succinatiphilus (species), Atopobium sp. F0209 (species), Corynebacterium freiburgense (species), Lactobacillus sp. Akhmrol (species), Anaerococcus sp. 9401487 (species), Mesorhizobium (genus), Lactobacillus reuteri (species), Megasphaera sp. UPII 199-6 (species), Lactobacillus sp. C30An8 (species), Peptococcus sp. S9 Pr-12 (species), Helcococcus seattlensis (species), Moraxellaceae (family), Moraxella (genus), Eikenella (genus), Eikenella corrodens (species), Vagococcus (genus), Phyllobacterium (genus), Veillonella dispar (species), Sutterella wadsworthensis (species), Johnsonella ignava (species), Bacteroides acidifaciens (species), Leptotrichia hofstadii (species), Leptotrichia shahii (species), Capnocytophaga sp. AHN9756 (species), Bergeyella sp. AF14 (species), Olsenella sp. F0004 (species), Bacteroides sp. D22 (species), Phyllobacterium sp. T50 (species), Actinomyces sp. ICM47 (species), Fusobacterium sp. AS2 (species), Leptotrichiaceae (family), Comamonas (genus), Peptostreptococcus (genus), Actinomyces viscosus (species), Actinomyces odontolyticus (species), Bifidobacterium (genus), Bifidobacteriaceae (family), Rhodospirillaceae (family), Bifidobacteriales (order), Roseburia intestinalis (species), Thalassospira (genus), Bifidobacterium longum (species), Aggregatibacter (genus), Streptococcus sp. 11aTha1 (species), Sutterellaceae (family), Flavobacterium (genus), Cronobacter sakazakii (species), Anaerococcus vaginalis (species), Sphingobacteriia (class), Brucellaceae (family), Sphingobacteriales (order), Akkermansia (genus), Peptoniphilus sp. gpac018A (species), Citrobacter sp. BW4 (species), Cronobacter (genus), Corynebacterium sp. jw37 (species), Staphylococcus aureus (species), Brevundimonas (genus), Caulobacteraceae (family), Caulobacterales (order), Anaerobacillus alkalidiazotrophicus (species), Anaerobacillus (genus), Acinetobacter sp. WB22-23 (species), Pseudomonas (genus), Neisseriaceae (family), Parabacteroides distasonis (species), Prevotella (genus), Faecalibacterium prausnitzii (species), Streptococcus parasanguinis (species), Cutibacterium acnes (species), Veillonellaceae (family), Leptotrichia (genus), Phascolarctobacterium (genus), Flavobacteriaceae (family), Delftia (genus), Flavobacteriia (class), Prevotellaceae (family), Lachnospiraceae (family), Peptostreptococcaceae (family), Dorea (genus), Flavobacteriales (order), Neisseriales (order), Parabacteroides (genus), Streptococcus sp. oral taxon G63 (species), Acidaminococcaceae (family), Veillonella sp. CM60 (species), Staphylococcus sp. C912 (species), Fusicatenibacter saccharivorans (species), Fusicatenibacter (genus), Staphylococcus sp. 334802 (species), Parabacteroides merdae (species), Collinsella aerofaciens (species), Peptoniphilus sp. 1-14 (species), Propionibacterium sp. KPL1844 (species), Methylobacterium longum (species), and Staphylococcus sp. C5116 (species);
generating a second site-specific appendix-related characterization model based on the second site-specific composition features;
collecting a user sample from an additional user, the user sample associated with the at least one of the skin site, the genital site, the mouth site, and the nose site; and
determining an additional appendix-related characterization for the additional user for the appendix-related condition based on the second site-specific appendix-related characterization model.

5. The method of claim 1, wherein the samples comprise site-specific samples associated with a skin site, wherein determining the set of microbiome features comprises determining the set of microbiome composition features comprising site-specific composition features associated with the skin site and at least one of Pseudomonas (genus), Neisseriaceae (family), Parabacteroides distasonis (species), Prevotella (genus), Faecalibacterium prausnitzii (species), Streptococcus parasanguinis (species), Cutibacterium acnes (species), Veillonellaceae (family), Leptotrichia (genus), Phascolarctobacterium (genus), Flavobacteriaceae (family), Delftia (genus), Flavobacteriia (class), Prevotellaceae (family), Lachnospiraceae (family), Peptostreptococcaceae (family), Dorea (genus), Flavobacteriales (order), Neisseriales (order), Parabacteroides (genus), Streptococcus sp. oral taxon G63 (species), Acidaminococcaceae (family), Veillonella sp. CM60 (species), Staphylococcus sp. C912 (species), Leptotrichiaceae (family), Fusicatenibacter saccharivorans (species), Fusicatenibacter (genus), Staphylococcus sp. 334802 (species), Parabacteroides merdae (species), Collinsella aerofaciens (species), Sphingobacteriia (class), Sphingobacteriales (order), Peptoniphilus sp. 1-14 (species), Anaerobacillus (genus), Propionibacterium sp. KPL1844 (species), Methylobacterium longum (species), and Staphylococcus sp. C5116 (species), wherein generating the appendix-related characterization model comprises generating a site-specific appendix-related characterization model based on the supplementary data and the site-specific composition features, and wherein determining the appendix-related characterization comprises determining the appendix-related characterization for the user for the appendix-related condition based on the site-specific appendix-related characterization model.

6. The method of claim 1, wherein the samples comprise site-specific samples associated with a genital site, wherein determining the set of microbiome features comprises determining the set of microbiome composition features comprising site-specific composition features associated with the genital site and at least one of Gemella (genus), Veillonella atypica (species), Dialister pneumosintes (species), Lactobacillus crispatus (species), Phyllobacteriaceae (family), Aquabacterium (genus), Anaeroglobus (genus), Anaeroglobus geminatus (species), Ochrobactrum (genus), Mobiluncus curtisii (species), Actinomyces neuii (species), Anaerococcus lactolyticus (species), Lactobacillus johnsonii (species), Verrucomicrobiales (order), Verrucomicrobia (phylum), Verrucomicrobiae (class), Verrucomicrobiaceae (family), Dialister succinatiphilus (species), Atopobium sp. F0209 (species), Corynebacterium freiburgense (species), Lactobacillus sp. Akhmrol (species), Anaerococcus sp. 9401487 (species), Mesorhizobium (genus), Lactobacillus reuteri (species), Megasphaera sp. UPII 199-6 (species), Lactobacillus sp. C30An8 (species), Peptococcus sp. S9 Pr-12 (species), and Helcococcus seattlensis (species), wherein generating the appendix-related characterization model comprises generating a site-specific appendix-related characterization model based on the supplementary data and the site-specific composition features, and wherein determining the appendix-related characterization comprises determining the appendix-related characterization for the user for the appendix-related condition based on the site-specific appendix-related characterization model.

7. The method of claim 1, wherein the samples comprise site-specific samples associated with a mouth site, wherein determining the set of microbiome features comprises determining the set of microbiome composition features comprising site-specific composition features associated with the mouth site and at least one of Moraxellaceae (family), Moraxella (genus), Eikenella (genus), Eikenella corrodens (species), Vagococcus (genus), Phyllobacterium (genus), Veillonella dispar (species), Sutterella wadsworthensis (species), Johnsonella ignava (species), Bacteroides acidifaciens (species), Leptotrichia hofstadii (species), Leptotrichia shahii (species), Capnocytophaga sp. AHN9756 (species), Bergeyella sp. AF14 (species), Olsenella sp. F0004 (species), Bacteroides sp. D22 (species), Phyllobacterium sp. T50 (species), Actinomyces sp. ICM47 (species), Fusobacterium sp. AS2 (species), and Leptotrichiaceae (family), wherein generating the appendix-related characterization model comprises generating a site-specific appendix-related characterization model based on the supplementary data and the site-specific composition features, and wherein determining the appendix-related characterization comprises determining the appendix-related characterization for the user for the appendix-related condition based on the site-specific appendix-related characterization model.

8. The method of claim 1, wherein the samples comprise site-specific samples associated with a nose site, wherein determining the set of microbiome features comprises determining the set of microbiome composition features comprising site-specific composition features associated with the nose site and at least one of Comamonas (genus), Peptostreptococcus (genus), Actinomyces viscosus (species), Actinomyces odontolyticus (species), Bifidobacterium (genus), Bifidobacteriaceae (family), Rhodospirillaceae (family), Bifidobacteriales (order), Roseburia intestinalis (species), Thalassospira (genus), Bifidobacterium longum (species), Aggregatibacter (genus), Streptococcus sp. 11aTha1 (species), Sutterellaceae (family), Flavobacterium (genus), Ochrobactrum (genus), Cronobacter sakazakii (species), Anaerococcus vaginalis (species), Sphingobacteriia (class), Brucellaceae (family), Sphingobacteriales (order), Akkermansia (genus), Peptoniphilus sp. gpac018A (species), Citrobacter sp. BW4 (species), Cronobacter (genus), Corynebacterium sp. jw37 (species), Staphylococcus aureus (species), Brevundimonas (genus), Caulobacteraceae (family), Caulobacterales (order), Anaerobacillus alkalidiazotrophicus (species), Anaerobacillus (genus), and Acinetobacter sp. WB22-23 (species), wherein generating the appendix-related characterization model comprises generating a site-specific appendix-related characterization model based on the supplementary data and the site-specific composition features, and wherein determining the appendix-related characterization comprises determining the appendix-related characterization for the user for the appendix-related condition based on the site-specific appendix-related characterization model.

9. The method of claim 1, wherein determining the set of microbiome features comprises determining the set of microbiome composition features associated with at least one of Enterococcus raffinosus (species), Staphylococcus sp. C912 (species), Gemella sp. 933-88 (species), Veillonella (genus), Gammaproteobacteria (class), Enterococcus sp. SI-4 (species), Enterobacteriales (order), Enterobacteriaceae (family), Phascolarctobacterium (genus), Odoribacter (genus), Ruminococcaceae (family), Acidaminococcaceae (family), Bilophila sp. 4_1_30 (species), Anaerostipes sp. 5_1_63FAA (species), Desulfovibrionaceae (family), Phascolarctobacterium faecium (species), Desulfovibrionales (order), Faecalibacterium (genus), Deltaproteobacteria (class), Burkholderiaceae (family), Alistipes sp. RMA 9912 (species), Methanobrevibacter (genus), Odoribacter splanchnicus (species), Alistipes sp. HGB5 (species), Gemella (genus), Subdoligranulum variabile (species), Methanobrevibacter smithii (species), Intestinimonas (genus), Lactobacillus sp. 7_1_47FAA (species), Methanobacteriaceae (family), Bilophila (genus), Methanobacteriales (order), Clostridiaceae (family), Euryarchaeota (phylum), Methanobacteria (class), Flavonifractor plautii (species), Carnobacteriaceae (family), Kluyvera (genus), Kluyvera georgiana (species), Blautia faecis (species), Faecalibacterium prausnitzii (species), Lactonifactor longoviformis (species), Roseburia sp. 11SE39 (species), Bacteroides sp. AR29 (species), Collinsella (genus), Alistipes sp. NML05A004 (species), Prevotella timonensis (species), Anaerostipes (genus), Lactonifactor (genus), Anaerostipes sp. 3_2_56FAA (species), Coriobacteriaceae (family), Klebsiella sp. SOR89 (species), Megasphaera sp. DNF00912 (species), Veillonella dispar (species), Lactobacillus mucosae (species), Bacteroides fragilis (species), Streptococcus equinus (species), Bacteroides plebeius (species), Propionibacterium sp. MSP09A (species), Streptococcus pasteurianus (species), Anaerovibrio sp. 765 (species), Akkermansia muciniphila (species), Actinomyces turicensis (species), Cronobacter sakazakii (species), Veillonella rogosae (species), Blautia glucerasea (species), Acidaminococcus intestini (species), Propionibacterium granulosum (species), Bacteroides thetaiotaomicron (species), Fusobacterium sp. CM21 (species), Pediococcus sp. MFC1 (species), Turicibacter sanguinis (species), Sarcina ventriculi (species), Megasphaera genomosp. C1 (species), Streptococcus sp. BS35a (species), Streptococcus thermophilus (species), Fusobacterium ulcerans (species), Morganella morganii (species), Bacteroides sp. SLC1-38 (species), Bacteroides eggerthii (species), Bacteroides coprocola (species), Bacteroides sp. CB57 (species), Bifidobacterium stercoris (species), Veillonella atypica (species), Fusobacterium necrogenes (species), Lactobacillus crispatus (species), Veillonella sp. MSA12 (species), Asaccharospora irregularis (species), Erysipelatoclostridium ramosum (species), Lactobacillus sp. TAB-22 (species), Parasutterella excrementihominis (species), Lactobacillus sp. C412 (species), Parabacteroides sp. 157 (species), Klebsiella (genus), Epulopiscium (genus), Streptococcus (genus), Propionibacterium (genus), Cronobacter (genus), Anaerovibrio (genus), Intestinibacter (genus), Staphylococcus (genus), Turicibacter (genus), Alloprevotella (genus), Pediococcus (genus), Morganella (genus), Acidaminococcus (genus), Succinivibrio (genus), Anaerofilum (genus), Megasphaera (genus), Asaccharospora (genus), Butyrivibrio (genus), Finegoldia (genus), Anaerococcus (genus), Streptococcaceae (family), Propionibacteriaceae (family), Veillonellaceae (family), Staphylococcaceae (family), Sphingobacteriaceae (family), Clostridiales Family XI. Incertae Sedis (family), Peptostreptococcaceae (family), Succinivibrionaceae (family), Dermabacteraceae (family), Corynebacteriaceae (family), Rhodospirillaceae (family), Selenomonadales (order), Lactobacillales (order), Clostridiales (order), Xanthomonadales (order), Bacillales (order), Pleurocapsales (order), Aeromonadales (order), Pseudomonadales (order), Bacilli (class), Negativicutes (class), Clostridia (class), Proteobacteria (phylum), Cyanobacteria (phylum), Bacteroides finegoldii (species), Alistipes putredinis (species), Actinobacteria (class), Lactobacillaceae (family), Bifidobacteriaceae (family), Bifidobacterium (genus), Bifidobacteriales (order), and Oscillospiraceae (family), and wherein generating the appendix-related characterization model comprises generating the appendix-related characterization model based on the supplementary data and the set of microbiome composition features.

10. The method of claim 1, wherein determining the microorganism sequence dataset comprises determining at least one of a metagenomic library and a metatranscriptomic library based on at least a subset of the microorganism nucleic acids, and wherein determining the set of microbiome features comprises determining the set of microbiome features based on the at least one of the metagenomic library and the metatranscriptomic library.

11. The method of claim 1, wherein determining the set of microbiome features comprises applying a set of analytical techniques to determine at least one of presence of at least one of a microbiome composition diversity feature and a microbiome functional diversity feature, absence of the at least one of the microbiome composition diversity feature and the microbiome functional diversity feature, a relative abundance feature describing relative abundance of different taxonomic groups associated with the appendix-related condition, a ratio feature describing a ratio between at least two microbiome features associated with the different taxonomic groups, an interaction feature describing an interaction between the different taxonomic groups, and a phylogenetic distance feature describing phylogenetic distance between the different taxonomic groups, based on the microorganism sequence dataset, and wherein the set of analytical techniques comprises at least one of a univariate statistical test, a multivariate statistical test, a dimensionality reduction technique, and an artificial intelligence approach.

12. The method of claim 1, wherein the therapy comprises at least one of a consumable, a device-related therapy, a surgical operation, a psychological-associated therapy, and a behavior modification therapy, and wherein providing the therapy comprises providing a recommendation for the therapy to the user at a computing device associated with the user.

13. A method for characterizing an appendix-related condition associated with microorganisms, the method comprising:
collecting a sample from a user, wherein the sample comprises microorganism nucleic acids corresponding to the microorganisms associated with the appendix-related condition;
determining a microorganism dataset associated with the user based on the microorganism nucleic acids of the sample;
determining user microbiome features comprising at least one of user microbiome composition features and user microbiome functional features, based on the microorganism dataset, wherein the user microbiome features are associated with the appendix-related condition;
determining an appendix-related characterization for the user for the appendix-related condition based on the user microbiome features; and
facilitating therapeutic intervention in relation to a therapy for the user for facilitating improvement of the appendix-related condition, based on the appendix-related characterization.

14. The method of claim 13, wherein determining the user microbiome features comprises determining, based on the microorganism dataset, the user microbiome composition features comprising site-specific composition features associated with a gut site and at least one of Neisseriaceae (family), Neisseria mucosa (species), Aggregatibacter aphrophilus (species), Bacteroides uniformis (species), Bacteroides vulgatus (species), Parabacteroides distasonis (species), Megasphaera (genus), Proteobacteria (phylum), Micrococcaceae (family), Streptococcus thermophilus (species), Streptococcus parasanguinis (species), Gemella (genus), Clostridium (genus), Actinomyces odontolyticus (species), Actinomycetales (order), Actinomycetaceae (family), Betaproteobacteria (class), Gemella morbillorum (species), Rothia (genus), Lactobacillus crispatus (species), Pseudomonadales (order), Oxalobacteraceae (family), Burkholderiales (order), Gemella sp. 933-88 (species), Micrococcales (order), Bacteroides acidifaciens (species), Mogibacterium (genus), Bacteroides sp. AR20 (species), Bacteroides sp. AR29 (species), Burkholderiaceae (family), Erysipelotrichaceae (family), Xanthomonadales (order), Pseudomonadaceae (family), Actinomyces sp. oral strain Hal-1065 (species), Roseburia intestinalis (species), Porphyromonadaceae (family), Shuttleworthia (genus), Clostridia (class), Clostridiales (order), Peptostreptococcaceae (family), Peptococcaceae (family), Carnobacteriaceae (family), Dialister sp. E2_20 (species), Neisseriales (order), Megasphaera genomosp. C1 (species), Moryella (genus), Synergistetes (phylum), Erysipelotrichia (class), Erysipelotrichales (order), Clostridiales Family XIII. Incertae Sedis (family), Roseburia sp. 11SE39 (species), Bacteroides sp. D22 (species), Synergistia (class), Synergistales (order), Synergistaceae (family), Lactobacillus sp. TAB-22 (species), Flavonifractor (genus), Sutterellaceae (family), Anaerostipes sp. 5_1_63FAA (species), Streptococcus sp. 2011_Oral_MS_A3 (species), Veillonella sp. 2011_Oral_VSA_D3 (species), Finegoldia sp. S9 AA1-5 (species), Fretibacterium (genus), Staphylococcus sp. 334802 (species), Peptoclostridium (genus), Intestinibacter (genus), Acinetobacter (genus), Klebsiella (genus), Bacteroides thetaiotaomicron (species), Butyrivibrio (genus), Fusobacterium necrogenes (species), Herbaspirillum (genus), Herbaspirillum seropedicae (species), Pediococcus (genus), Finegoldia magna (species), Blautia hansenii (species), Enterococcus faecalis (species), Lactococcus lactis (species), Bacillus (genus), Clostridioides difficile (species), Blautia coccoides (species), Erysipelatoclostridium ramosum (species), Weissella confusa (species), Lactobacillus plantarum (species), Lactobacillus paracasei (species), Bifidobacterium adolescentis (species), Bifidobacterium breve (species), Bifidobacterium dentium (species), Bifidobacterium animalis (species), Bifidobacterium pseudocatenulatum (species), Bacteroides ovatus (species), Peptoniphilus lacrimalis (species), Anaerococcus vaginalis (species), Rahnella (genus), Bilophila wadsworthia (species), Sneathia sanguinegens (species), Succiniclasticum (genus), Sporobacter (genus), Pseudobutyrivibrio ruminis (species), Weissella (genus), Bacteroides stercoris (species), Lactobacillus rhamnosus (species), Pantoea (genus), Holdemania (genus), Holdemania filiformis (species), Thermoanaerobacterales (order), Bifidobacterium gallicum (species), Bifidobacterium pullorum (species), Leuconostocaceae (family), Eggerthella lenta (species), Papillibacter (genus), Anaerostipes caccae (species), Pseudoflavonifractor capillosus (species), Anaerovorax (genus), Parasporobacterium (genus), Parasporobacterium paucivorans (species), Oscillospira (genus), Oscillospira guilliermondii (species), Actinomyces turicensis (species), Anaerosinus (genus), Sneathia (genus), Brevibacterium paucivorans (species), Lactobacillus sp. CR-609S (species), Thermoanaerobacteraceae (family), Bacillaceae (family), Gelria (genus), Acidobacteriales (order), Bacteroides massiliensis (species), Rhodocyclales (order), Anaerofustis stercorihominis (species), Alistipes finegoldii (species), Oscillospiraceae (family), Peptoniphilus sp. 2002-38328 (species), Hespellia (genus), Bacteroides sp. 35AE37 (species), Marvinbryantia (genus), Anaerosporobacter mobilis (species), Anaerofustis (genus), Catabacter (genus), Flavonifractor plautii (species), Proteiniphilum (genus), Roseburia faecis (species), Streptococcus sp. 816-11 (species), Bacteroides sp. 4072 (species), Alistipes shahii (species), Bacteroides intestinalis (species), Lactonifactor longoviformis (species), Bifidobacterium tsurumiense (species), Bacteroides dorei (species), Bacteroides xylanisolvens (species), Cronobacter (genus), Alloscardovia (genus), Alloscardovia omnicolens (species), Lactonifactor (genus), Catabacteriaceae (family), Adlercreutzia equolifaciens (species), Adlercreutzia (genus), Alistipes sp. EBA6-25c12 (species), Bacteroides sp. EBA5-17 (species), Oscillibacter (genus), Gordonibacter pamelaeae (species), Alistipes sp. NML05A004 (species), Parasutterella excrementihominis (species), Mitsuokella sp. DJF_RR21 (species), Butyricimonas (genus), Bifidobacterium stercoris (species), Alistipes indistinctus (species), Gordonibacter (genus), Anaerostipes hadrus (species), Klebsiella sp. B12 (species), Alistipes sp. RMA 9912 (species), Anaerosporobacter (genus), Bacteroides faecis (species), Blautia sp. Ser5 (species), Bacteroides chinchillae (species), Bilophila sp. 4_1_30 (species), Caldicoprobacteraceae (family), Enterobacter sp. UDC345 (species), Bifidobacterium biavatii (species), Peptoniphilus sp. 1-14 (species), Alistipes sp. HGB5 (species), Bacteroides sp. SLC1-38 (species), Lactobacillus sp. Akhmrol (species), Klebsiella sp. SOR89 (species), Enterococcus sp. C6 I11 (species), Pseudoflavonifractor (genus), Bacteroides sp. dnLKV9 (species), Megasphaera sp. BV3C16-1 (species), Faecalibacterium sp. canine oral taxon 147 (species), Varibaculum sp. CCUG 45114 (species), Butyricimonas sp. 214-4 (species), Anaerostipes rhamnosivorans (species), Negativicoccus sp. S5-A15 (species), [Collinsella] massiliensis (species), Corynebacterium sp. jw37 (species), Roseburia sp. 499 (species), Dialister sp. S7MSR5 (species), Anaerococcus sp. S8 87-3 (species), Finegoldia sp. S8 F7 (species), Murdochiella sp. S9 PR-10 (species), Peptoniphilus sp. S9 PR-13 (species), Bacteroides sp. J1511 (species), Corynebacterium sp. 713182/2012 (species), Rahnella sp. BSP18 (species), Intestinimonas (genus), Robinsoniella sp. KNHs210 (species), Candidatus Soleaferrea (genus), Butyricimonas faecihominis (species), Senegalimassilia (genus), Peptoniphilus sp. DNF00840 (species), Romboutsia (genus), and Coprobacter secundus (species), wherein determining the appendix-related characterization comprises determining the appendix-related characterization for the user for the appendix-related condition based on the site-specific composition features.

15. The method of claim 13, wherein determining the user microbiome features comprises determining, based on the microorganism dataset, the user microbiome composition features comprising site-specific composition features associated with a skin site and at least one of Pseudomonas (genus), Neisseriaceae (family), Parabacteroides distasonis (species), Prevotella (genus), Faecalibacterium prausnitzii (species), Streptococcus parasanguinis (species), Cutibacterium acnes (species), Veillonellaceae (family), Leptotrichia (genus), Phascolarctobacterium (genus), Flavobacteriaceae (family), Delftia (genus), Flavobacteriia (class), Prevotellaceae (family), Lachnospiraceae (family), Peptostreptococcaceae (family), Dorea (genus), Flavobacteriales (order), Neisseriales (order), Parabacteroides (genus), Streptococcus sp. oral taxon G63 (species), Acidaminococcaceae (family), Veillonella sp. CM60 (species), Staphylococcus sp. C912 (species), Leptotrichiaceae (family), Fusicatenibacter saccharivorans (species), Fusicatenibacter (genus), Staphylococcus sp. 334802 (species), Parabacteroides merdae (species), Collinsella aerofaciens (species), Sphingobacteriia (class), Sphingobacteriales (order), Peptoniphilus sp. 1-14 (species), Anaerobacillus (genus), Propionibacterium sp. KPL1844 (species), Methylobacterium longum (species), and Staphylococcus sp. C5I16 (species), wherein determining the appendix-related characterization comprises determining the appendix-related characterization for the user for the appendix-related condition based on the site-specific composition features.

16. The method of claim 13, wherein determining the user microbiome features comprises determining, based on the microorganism dataset, the user microbiome composition features comprising site-specific composition features associated with a genital site and at least one of Gemella (genus), Veillonella atypica (species), Dialister pneumosintes (species), Lactobacillus crispatus (species), Phyllobacteriaceae (family), Aquabacterium (genus), Anaeroglobus (genus), Anaeroglobus geminatus (species), Ochrobactrum (genus), Mobiluncus curtisii (species), Actinomyces neuii (species), Anaerococcus lactolyticus (species), Lactobacillus johnsonii (species), Verrucomicrobiales (order), Verrucomicrobia (phylum), Verrucomicrobiae (class), Verrucomicrobiaceae (family), Dialister succinatiphilus (species), Atopobium sp. F0209 (species), Corynebacterium freiburgense (species), Lactobacillus sp. Akhmrol (species), Anaerococcus sp. 9401487 (species), Mesorhizobium (genus), Lactobacillus reuteri (species), Megasphaera sp. UPII 199-6 (species), Lactobacillus sp. C30An8 (species), Peptococcus sp. S9 Pr-12 (species), and Helcococcus seattlensis (species), wherein determining the appendix-related characterization comprises determining the appendix-related characterization for the user for the appendix-related condition based on the site-specific composition features.

17. The method of claim 13, wherein determining the user microbiome features comprises determining, based on the microorganism dataset, the user microbiome composition features comprising site-specific composition features associated with a mouth site and at least one of Moraxellaceae (family), Moraxella (genus), Eikenella (genus), Eikenella corrodens (species), Vagococcus (genus), Phyllobacterium (genus), Veillonella dispar (species), Sutterella wadsworthensis (species), Johnsonella ignava (species), Bacteroides acidifaciens (species), Leptotrichia hofstadii (species), Leptotrichia shahii (species), Capnocytophaga sp. AHN9756 (species), Bergeyella sp. AF14 (species), Olsenella sp. F0004 (species), Bacteroides sp. D22 (species), Phyllobacterium sp. T50 (species), Actinomyces sp. ICM47 (species), Fusobacterium sp. AS2 (species), and Leptotrichiaceae (family), wherein determining the appendix-related characterization comprises determining the appendix-related characterization for the user for the appendix-related condition based on the site-specific composition features.

18. The method of claim 13, wherein determining the user microbiome features comprises determining, based on the microorganism dataset, the user microbiome composition features comprising site-specific composition features associated with a nose site and at least one of Comamonas (genus), Peptostreptococcus (genus), Actinomyces viscosus (species), Actinomyces odontolyticus (species), Bifidobacterium (genus), Bifidobacteriaceae (family), Rhodospirillaceae (family), Bifidobacteriales (order), Roseburia intestinalis (species), Thalassospira (genus), Bifidobacterium longum (species), Aggregatibacter (genus), Streptococcus sp. 11aTha1 (species), Sutterellaceae (family), Flavobacterium (genus), Ochrobactrum (genus), Cronobacter sakazakii (species), Anaerococcus vaginalis (species), Sphingobacteriia (class), Brucellaceae (family), Sphingobacteriales (order), Akkermansia (genus), Peptoniphilus sp. gpac018A (species), Citrobacter sp. BW4 (species), Cronobacter (genus), Corynebacterium sp. jw37 (species), Staphylococcus aureus (species), Brevundimonas (genus), Caulobacteraceae (family), Caulobacterales (order), Anaerobacillus alkalidiazotrophicus (species), Anaerobacillus (genus), and Acinetobacter sp. WB22-23 (species), wherein determining the appendix-related characterization comprises determining the appendix-related characterization for the user for the appendix-related condition based on the site-specific composition features.

19. The method of claim 13, wherein determining the user microbiome features comprises determining, based on the microorganism dataset, the user microbiome functional features associated with at least one of Neurodegenerative Disease, Signaling Molecules and Interaction, Xenobiotics Biodegradation and Metabolism, Ascorbate and aldarate metabolism, Huntington's disease, Inositol phosphate metabolism, Propanoate metabolism, Starch and sucrose metabolism, Caprolactam degradation, Cell motility and secretion, Valine, leucine and isoleucine degradation, Tryptophan metabolism, Type I diabetes mellitus, Phenylalanine metabolism, Selenocompound metabolism, Lysine degradation, Polycyclic aromatic hydrocarbon degradation, Glycan biosynthesis and metabolism, Renal cell carcinoma, Butanoate metabolism, Carbon fixation pathways in prokaryotes, Citrate cycle (TCA cycle), Lipopolysaccharide biosynthesis, RNA transport, Thiamine metabolism, 1,1,1-Trichloro-2,2-bis (4-chlorophenyl)ethane (DDT) degradation, Electron transfer carriers, Amyotrophic lateral sclerosis (ALS), Prion disease, Toluene degradation, alpha-Linolenic acid metabolism, [V] Defense mechanisms, [0] Post-translational modification, protein turnover, and chaperones, [R] General function prediction only, [I] Lipid transport and metabolism, [H] Coenzyme transport and metabolism, Energy Metabolism, Nervous System, Signal Transduction, Cellular Processes and Signaling, Translation, Metabolism, Cell Growth and Death, Endocrine System, Amino Acid Metabolism, Metabolism of Cofactors and Vitamins, Replication and Repair, Metabolism of Terpenoids and Polyketides, Infectious Diseases, Amino acid related enzymes, Photosynthesis, Pantothenate and CoA biosynthesis, Photosynthesis proteins, Glutamatergic synapse, Tuberculosis, Two-component system, Aminoacyl-tRNA biosynthesis, Ribosome, Other ion-coupled transporters, Terpenoid backbone biosynthesis, Cell cycle—Caulobacter, Other transporters, Base excision repair, Peptidoglycan biosynthesis, Vibrio cholerae pathogenic cycle, Limonene and pinene degradation, Secretion system, Nucleotide excision repair, Translation factors, Alanine, aspartate and glutamate metabolism, Ribosome Biogenesis, Others (KEGG3), Ribosome biogenesis in eukaryotes, Polyketide sugar unit biosynthesis, Streptomycin biosynthesis, Homologous recombination, Oxidative phosphorylation, Function unknown, Carbon fixation in photosynthetic organisms, Cytoskeleton proteins, DNA repair and recombination proteins, Inorganic ion transport and metabolism, Amino acid metabolism, Geraniol degradation, Protein export, Phenylalanine, tyrosine and tryptophan biosynthesis, Lysine biosynthesis, Ethylbenzene degradation, Transcription machinery, RNA polymerase, Biosynthesis of vancomycin group antibiotics, Mismatch repair, Naphthalene degradation, Pyrimidine metabolism, D-Glutamine and D-glutamate metabolism, Zeatin biosynthesis, K02004 (KEGG4), and K03100 (KEGG4), wherein determining the appendix-related characterization comprises determining the appendix-related characterization for the user for the appendix-related condition based on the user microbiome functional features.

20. The method of claim 13, wherein the therapy comprises at least one of a probiotic therapy and a prebiotic therapy, wherein facilitating therapeutic intervention comprises promoting the at least one of the probiotic therapy and the prebiotic therapy to the user for facilitating improvement of the appendix-related condition, and wherein the at least one of the probiotic therapy and the prebiotic therapy is associated with at least one of Enterococcus raffinosus, Staphylococcus sp. C912, Gemella sp. 933-88, Enterococcus sp. SI-4, Bilophila sp. 4_1_30, Anaerostipes sp. 5_1_63FAA, Phascolarctobacterium faecium, Alistipes sp. RMA 9912, Odoribacter splanchnicus, Alistipes sp. HGB5, Subdoligranulum variabile, Methanobrevibacter smithii, Lactobacillus sp. 7_1_47FAA, Flavonifractor plautii, Kluyvera georgiana, Blautia faecis, Faecalibacterium prausnitzii, Lactonifactor longoviformis, Roseburia sp. 11SE39, Bacteroides sp. AR29, Alistipes sp. NML05A004, Prevotella timonensis, Anaerostipes sp. 3_2_56FAA, Klebsiella sp. SOR89, Megasphaera sp. DNF00912, Veillonella dispar, Lactobacillus mucosae, Bacteroides fragilis, Streptococcus equinus, Bacteroides plebeius, Propionibacterium sp. MSP09A, Streptococcus pasteurianus, Anaerovibrio sp. 765, Akkermansia muciniphila, Actinomyces turicensis, Cronobacter sakazakii, Veillonella rogosae, Blautia glucerasea, Acidaminococcus intestini, Propionibacterium granulosum, Bacteroides thetaiotaomicron, Fusobacterium sp. CM21, Pediococcus sp. MFC1, Turicibacter sanguinis, Sarcina ventriculi, Megasphaera genomosp. C1, Streptococcus sp. BS35a, Streptococcus thermophilus, Fusobacterium ulcerans, Morganella morganii, Bacteroides sp. SLC1-38, Bacteroides eggerthii, Bacteroides coprocola, Bacteroides sp. CB57, Bifidobacterium stercoris, Veillonella atypica, Fusobacterium necrogenes, Lactobacillus crispatus, Veillonella sp. MSA12, Asaccharospora irregularis, Erysipelatoclostridium ramosum, Lactobacillus sp. TAB-22, Parasutterella excrementihominis, Lactobacillus sp. C412, Parabacteroides sp. 157, Bacteroides finegoldii, and Alistipes putredinis.

21. A method for characterizing an appendix-related condition associated with microorganisms, the method comprising:
collecting a sample from a user, wherein the sample comprises microorganism nucleic acids corresponding to the microorganisms associated with the appendix-related condition;
determining a microorganism dataset associated with the user based on the microorganism nucleic acids of the sample;
determining user microbiome features based on the microorganism dataset, wherein the user microbiome features are associated with the appendix-related condition; and
determining an appendix-related characterization for the user for the appendix-related condition based on the user microbiome features.

22. The method of claim 21, wherein the user microbiome features comprise user microbiome composition features associated with at least one of Gemella (genus), Veillonella atypica (species), Dialister pneumosintes (species), Lactobacillus crispatus (species), Phyllobacteriaceae (family), Aquabacterium (genus), Anaeroglobus (genus), Anaeroglobus geminatus (species), Ochrobactrum (genus), Mobiluncus curtisii (species), Actinomyces neuii (species), Anaerococcus lactolyticus (species), Lactobacillus johnsonii (species), Verrucomicrobiales (order), Verrucomicrobia (phylum), Verrucomicrobiae (class), Verrucomicrobiaceae (family), Dialister succinatiphilus (species), Atopobium sp. F0209 (species), Corynebacterium freiburgense (species), Lactobacillus sp. Akhmrol (species), Anaerococcus sp. 9401487 (species), Mesorhizobium (genus), Lactobacillus reuteri (species), Megasphaera sp. UPII 199-6 (species), Lactobacillus sp. C30An8 (species), Peptococcus sp. S9 Pr-12 (species), Helcococcus seattlensis (species), Neisseriaceae (family), Neisseria mucosa (species), Aggregatibacter aphrophilus (species), Bacteroides uniformis (species), Bacteroides vulgatus (species), Parabacteroides distasonis (species), Megasphaera (genus), Proteobacteria (phylum), Micrococcaceae (family), Streptococcus thermophilus (species), Streptococcus parasanguinis (species), Clostridium (genus), Actinomyces odontolyticus (species), Actinomycetales (order), Actinomycetaceae (family), Betaproteobacteria (class), Gemella morbillorum (species), Rothia (genus), Pseudomonadales (order), Oxalobacteraceae (family), Burkholderiales (order), Gemella sp. 933-88 (species), Micrococcales (order), Bacteroides acidifaciens (species), Mogibacterium (genus), Bacteroides sp. AR20 (species), Bacteroides sp. AR29 (species), Burkholderiaceae (family), Erysipelotrichaceae (family), Xanthomonadales (order), Pseudomonadaceae (family), Actinomyces sp. oral strain Hal-1065 (species), Roseburia intestinalis (species), Porphyromonadaceae (family), Shuttleworthia (genus), Clostridia (class), Clostridiales (order), Peptostreptococcaceae (family), Peptococcaceae (family), Carnobacteriaceae (family), Dialister sp. E2_20 (species), Neisseriales (order), Megasphaera genomosp. C1 (species), Moryella (genus), Synergistetes (phylum), Erysipelotrichia (class), Erysipelotrichales (order), Clostridiales Family XIII. Incertae Sedis (family), Roseburia sp. 11SE39 (species), Bacteroides sp. D22 (species), Synergistia (class), Synergistales (order), Synergistaceae (family), Lactobacillus sp. TAB-22 (species), Flavonifractor (genus), Sutterellaceae (family), Anaerostipes sp. 5_1_63FAA (species), Streptococcus sp. 2011_Oral_MS_A3 (species), Veillonella sp. 2011_Oral_VSA_D3 (species), Finegoldia sp. S9 AA1-5 (species), Fretibacterium (genus), Staphylococcus sp. 334802 (species), Peptoclostridium (genus), Intestinibacter (genus), Acinetobacter (genus), Klebsiella (genus), Bacteroides thetaiotaomicron (species), Butyrivibrio (genus), Fusobacterium necrogenes (species), Herbaspirillum (genus), Herbaspirillum seropedicae (species), Pediococcus (genus), Finegoldia magna (species), Blautia hansenii (species), Enterococcus faecalis (species), Lactococcus lactis (species), Bacillus (genus), Clostridioides difficile (species), Blautia coccoides (species), Erysipelatoclostridium ramosum (species), Weissella confusa (species), Lactobacillus plantarum (species), Lactobacillus paracasei (species), Bifidobacterium adolescentis (species), Bifidobacterium breve (species), Bifidobacterium dentium (species), Bifidobacterium animalis (species), Bifidobacterium pseudocatenulatum (species), Bacteroides ovatus (species), Peptoniphilus lacrimalis (species), Anaerococcus vaginalis (species), Rahnella (genus), Bilophila wadsworthia (species), Sneathia sanguinegens (species), Succiniclasticum (genus), Sporobacter (genus), Pseudobutyrivibrio ruminis (species), Weissella (genus), Bacteroides stercoris (species), Lactobacillus rhamnosus (species), Pantoea (genus), Holdemania (genus), Holdemania filiformis (species), Thermoanaerobacterales (order), Bifidobacterium gallicum (species), Bifidobacterium pullorum (species), Leuconostocaceae (family), Eggerthella lenta (species), Papillibacter (genus), Anaerostipes caccae (species), Pseudoflavonifractor capillosus (species), Anaerovorax (genus), Parasporobacterium (genus), Parasporobacterium paucivorans (species), Oscillospira (genus), Oscillospira guilliermondii (species), Actinomyces turicensis (species), Anaerosinus (genus), Sneathia (genus), Brevibacterium paucivorans (species), Lactobacillus sp. CR-609S (species), Thermoanaerobacteraceae (family), Bacillaceae (family), Gelria (genus), Acidobacteriales (order), Bacteroides massiliensis (species), Rhodocyclales (order), Anaerofustis stercorihominis (species), Alistipes finegoldii (species), Oscillospiraceae (family), Peptoniphilus sp. 2002-38328 (species), Hespellia (genus), Bacteroides sp. 35AE37 (species), Marvinbryantia (genus), Anaerosporobacter mobilis (species), Anaerofustis (genus), Catabacter (genus), Flavonifractor plautii (species), Proteiniphilum (genus), Roseburia faecis (species), Streptococcus sp. S16-11 (species), Bacteroides sp. 4072 (species), Alistipes shahii (species), Bacteroides intestinalis (species), Lactonifactor longoviformis (species), Bifidobacterium tsurumiense (species), Bacteroides dorei (species), Bacteroides xylanisolvens (species), Cronobacter (genus), Alloscardovia (genus), Alloscardovia omnicolens (species), Lactonifactor (genus), Catabacteriaceae (family), Adlercreutzia equolifaciens (species), Adlercreutzia (genus), Alistipes sp. EBA6-25c12 (species), Bacteroides sp. EBA5-17 (species), Oscillibacter (genus), Gordonibacter pamelaeae (species), Alistipes sp. NML05A004 (species), Parasutterella excrementihominis (species), Mitsuokella sp. DJF_RR21 (species), Butyricimonas (genus), Bifidobacterium stercoris (species), Alistipes indistinctus (species), Gordonibacter (genus), Anaerostipes hadrus (species), Klebsiella sp. B12 (species), Alistipes sp. RMA 9912 (species), Anaerosporobacter (genus), Bacteroides faecis (species), Blautia sp. Ser5 (species), Bacteroides chinchillae (species), Bilophila sp. 4_1_30 (species), Caldicoprobacteraceae (family), Enterobacter sp. UDC345 (species), Bifidobacterium biavatii (species), Peptoniphilus sp. 1-14 (species), Alistipes sp. HGB5 (species), Bacteroides sp. SLC1-38 (species), Klebsiella sp. SOR89 (species), Enterococcus sp. C6 I11 (species), Pseudoflavonifractor (genus), Bacteroides sp. dnLKV9 (species), Megasphaera sp. BV3C16-1 (species), Faecalibacterium sp. canine oral taxon 147 (species), Varibaculum sp. CCUG 45114 (species), Butyricimonas sp. 214-4 (species), Anaerostipes rhamnosivorans (species), Negativicoccus sp. S5-A15 (species), [Collinsella] massiliensis (species), Corynebacterium sp. jw37 (species), Roseburia sp. 499 (species), Dialister sp. S7MSR5 (species), Anaerococcus sp. S8 87-3 (species), Finegoldia sp. S8 F7 (species), Murdochiella sp. S9 PR-10 (species), Peptoniphilus sp. S9 PR-13 (species), Bacteroides sp. J1511 (species), Corynebacterium sp. 713182/2012 (species), Rahnella sp. BSP18 (species), Intestinimonas (genus), Robinsoniella sp. KNHs210 (species), Candidatus Soleaferrea (genus), Butyricimonas faecihominis (species), Senegalimassilia (genus), Peptoniphilus sp. DNF00840 (species), Romboutsia (genus), Coprobacter secundus (species), Moraxellaceae (family), Moraxella (genus), Eikenella (genus), Eikenella corrodens (species), Vagococcus (genus), Phyllobacterium (genus), Veillonella dispar (species), Sutterella wadsworthensis (species), Johnsonella ignava (species), Leptotrichia hofstadii (species), Leptotrichia shahii (species), Capnocytophaga sp. AHN9756 (species), Bergeyella sp. AF14 (species), Olsenella sp. F0004 (species), Phyllobacterium sp. T50 (species), Actinomyces sp. ICM47 (species), Fusobacterium sp. AS2 (species), Leptotrichiaceae (family), Comamonas (genus), Peptostreptococcus (genus), Actinomyces viscosus (species), Bifidobacterium (genus), Bifidobacteriaceae (family), Rhodospirillaceae (family), Bifidobacteriales (order), Thalassospira (genus), Bifidobacterium longum (species), Aggregatibacter (genus), Streptococcus sp. 11aTha1 (species), Flavobacterium (genus), Cronobacter sakazakii (species), Sphingobacteriia (class), Brucellaceae (family), Sphingobacteriales (order), Akkermansia (genus), Peptoniphilus sp. gpac018A (species), Citrobacter sp. BW4 (species), Staphylococcus aureus (species), Brevundimonas (genus), Caulobacteraceae (family), Caulobacterales (order), Anaerobacillus alkalidiazotrophicus (species), Anaerobacillus (genus), Acinetobacter sp. WB22-23 (species), Pseudomonas (genus), Prevotella (genus), Faecalibacterium prausnitzii (species), Cutibacterium acnes (species), Veillonellaceae (family), Leptotrichia (genus), Phascolarctobacterium (genus), Flavobacteriaceae (family), Delftia (genus), Flavobacteriia (class), Prevotellaceae (family), Lachnospiraceae (family), Dorea (genus), Flavobacteriales (order), Parabacteroides (genus), Streptococcus sp. oral taxon G63 (species), Acidaminococcaceae (family), Veillonella sp. CM60 (species), Staphylococcus sp. C912 (species), Fusicatenibacter saccharivorans (species), Fusicatenibacter (genus), Parabacteroides merdae (species), Collinsella aerofaciens (species), Propionibacterium sp. KPL1844 (species), Methylobacterium longum (species), Staphylococcus sp. C5116 (species), Enterococcus raffinosus (species), Veillonella (genus), Gammaproteobacteria (class), Enterococcus sp. SI-4 (species), Enterobacteriales (order), Enterobacteriaceae (family), Odoribacter (genus), Ruminococcaceae (family), Desulfovibrionaceae (family), Phascolarctobacterium faecium (species), Desulfovibrionales (order), Faecalibacterium (genus), Deltaproteobacteria (class), Methanobrevibacter (genus), Odoribacter splanchnicus (species), Subdoligranulum variabile (species), Methanobrevibacter smithii (species), Lactobacillus sp. 7_1_47FAA (species), Methanobacteriaceae (family), Bilophila (genus), Methanobacteriales (order), Clostridiaceae (family), Euryarchaeota (phylum), Methanobacteria (class), Kluyvera (genus), Kluyvera georgiana (species), Blautia faecis (species), Collinsella (genus), Prevotella timonensis (species), Anaerostipes (genus), Anaerostipes sp. 3_2_56FAA (species), Coriobacteriaceae (family), Megasphaera sp. DNF00912 (species), Lactobacillus mucosae (species), Bacteroides fragilis (species), Streptococcus equinus (species), Bacteroides plebeius (species), Propionibacterium sp. MSP09A (species), Streptococcus pasteurianus (species), Anaerovibrio sp. 765 (species), Akkermansia muciniphila (species), Veillonella rogosae (species), Blautia glucerasea (species), Acidaminococcus intestini (species), Propionibacterium granulosum (species), Fusobacterium sp. CM21 (species), Pediococcus sp. MFC1 (species), Turicibacter sanguinis (species), Sarcina ventriculi (species), Streptococcus sp. BS35a (species), Fusobacterium ulcerans (species), Morganella morganii (species), Bacteroides eggerthii (species), Bacteroides coprocola (species), Bacteroides sp. CB57 (species), Veillonella sp. MSA12 (species), Asaccharospora irregularis (species), Lactobacillus sp. C412 (species), Parabacteroides sp. 157 (species), Epulopiscium (genus), Streptococcus (genus), Propionibacterium (genus), Anaerovibrio (genus), Staphylococcus (genus), Turicibacter (genus), Alloprevotella (genus), Morganella (genus), Acidaminococcus (genus), Succinivibrio (genus), Anaerofilum (genus), Asaccharospora (genus), Finegoldia (genus), Anaerococcus (genus), Streptococcaceae (family), Propionibacteriaceae (family), Staphylococcaceae (family), Sphingobacteriaceae (family), Succinivibrionaceae (family), Dermabacteraceae (family), Corynebacteriaceae (family), Selenomonadales (order), Lactobacillales (order), Bacillales (order), Pleurocapsales (order), Aeromonadales (order), Bacilli (class), Negativicutes (class), Cyanobacteria (phylum), Bacteroides finegoldii (species), Alistipes putredinis (species), Actinobacteria (class), and Lactobacillaceae (family), and wherein determining the appendix-related characterization comprises determining the appendix-related condition for the user for the appendix-related condition based on the user microbiome composition features.

23. The method of claim 22,
wherein the sample is associated with a first body site comprising at least one of a gut site, a skin site, a genital site, a mouth site, and a nose site,
wherein the user microbiome composition features comprise site-specific composition features, each site-specific composition feature associated with the first body site,
wherein determining the appendix-related characterization comprises determining the appendix-related characterization for the user for the appendix-related condition based on the site-specific composition features, and
wherein the method further comprises providing a first site-specific therapy to the user for facilitating improvement of the appendix-related condition, based on the appendix-related characterization, wherein the first site-specific therapy is associated with the first body site.

24. The method of claim 23, further comprising:
collecting a post-therapy sample from the user after the providing of the first site-specific therapy, wherein the post-therapy sample is associated with a second body site comprising at least one of the gut site, the skin site, the genital site, the mouth site, and the nose site;
determining a post-therapy appendix-related characterization for the user for the appendix-related condition based on site-specific features associated with the second body site; and
providing a second site-specific therapy to the user for facilitating improvement of the appendix-related condition, based on the post-therapy appendix-related characterization, wherein the second site-specific therapy is associated with the second body site.

25. The method of claim 21, wherein determining the user microbiome features comprises determining, based on the microorganism dataset, site-specific composition features associated with a gut site and at least one of Neisseriaceae (family), Neisseria mucosa (species), Aggregatibacter aphrophilus (species), Bacteroides uniformis (species), Bacteroides vulgatus (species), Parabacteroides distasonis (species), Megasphaera (genus), Proteobacteria (phylum), Micrococcaceae (family), Streptococcus thermophilus (species), Streptococcus parasanguinis (species), Gemella (genus), Clostridium (genus), Actinomyces odontolyticus (species), Actinomycetales (order), Actinomycetaceae (family), Betaproteobacteria (class), Gemella morbillorum (species), Rothia (genus), Lactobacillus crispatus (species), Pseudomonadales (order), Oxalobacteraceae (family), Burkholderiales (order), Gemella sp. 933-88 (species), Micrococcales (order), Bacteroides acidifaciens (species), Mogibacterium (genus), Bacteroides sp. AR20 (species), Bacteroides sp. AR29 (species), Burkholderiaceae (family), Erysipelotrichaceae (family), Xanthomonadales (order), Pseudomonadaceae (family), Actinomyces sp. oral strain Hal-1065 (species), Roseburia intestinalis (species), Porphyromonadaceae (family), Shuttleworthia (genus), Clostridia (class), Clostridiales (order), Peptostreptococcaceae (family), Peptococcaceae (family), Carnobacteriaceae (family), Dialister sp. E2_20 (species), Neisseriales (order), Megasphaera genomosp. C1 (species), Moryella (genus), Synergistetes (phylum), Erysipelotrichia (class), Erysipelotrichales (order), Clostridiales Family XIII. Incertae Sedis (family), Roseburia sp. 11SE39 (species), Bacteroides sp. D22 (species), Synergistia (class), Synergistales (order), Synergistaceae (family), Lactobacillus sp. TAB-22 (species), Flavonifractor (genus), Sutterellaceae (family), Anaerostipes sp. 5_1_63FAA (species), Streptococcus sp. 2011_Oral_MS_A3 (species), Veillonella sp. 2011_Oral_VSA_D3 (species), Finegoldia sp. S9 AA1-5 (species), Fretibacterium (genus), Staphylococcus sp. 334802 (species), Peptoclostridium (genus), Intestinibacter (genus), Acinetobacter (genus), Klebsiella (genus), Bacteroides thetaiotaomicron (species), Butyrivibrio (genus), Fusobacterium necrogenes (species), Herbaspirillum (genus), Herbaspirillum seropedicae (species), Pediococcus (genus), Finegoldia magna (species), Blautia hansenii (species), Enterococcus faecalis (species), Lactococcus lactis (species), Bacillus (genus), Clostridioides difficile (species), Blautia coccoides (species), Erysipelatoclostridium ramosum (species), Weissella confusa (species), Lactobacillus plantarum (species), Lactobacillus paracasei (species), Bifidobacterium adolescentis (species), Bifidobacterium breve (species), Bifidobacterium dentium (species), Bifidobacterium animalis (species), Bifidobacterium pseudocatenulatum (species), Bacteroides ovatus (species), Peptoniphilus lacrimalis (species), Anaerococcus vaginalis (species), Rahnella (genus), Bilophila wadsworthia (species), Sneathia sanguinegens (species), Succiniclasticum (genus), Sporobacter (genus), Pseudobutyrivibrio ruminis (species), Weissella (genus), Bacteroides stercoris (species), Lactobacillus rhamnosus (species), Pantoea (genus), Holdemania (genus), Holdemania filiformis (species), Thermoanaerobacterales (order), Bifidobacterium gallicum (species), Bifidobacterium pullorum (species), Leuconostocaceae (family), Eggerthella lenta (species), Papillibacter (genus), Anaerostipes caccae (species), Pseudoflavonifractor capillosus (species), Anaerovorax (genus), Parasporobacterium (genus), Parasporobacterium paucivorans (species), Oscillospira (genus), Oscillospira guilliermondii (species), Actinomyces turicensis (species), Anaerosinus (genus), Sneathia (genus), Brevibacterium paucivorans (species), Lactobacillus sp. CR-609S (species), Thermoanaerobacteraceae (family), Bacillaceae (family), Gelria (genus), Acidobacteriales (order), Bacteroides massiliensis (species), Rhodocyclales (order), Anaerofustis stercorihominis (species), Alistipes finegoldii (species), Oscillospiraceae (family), Peptoniphilus sp. 2002-38328 (species), Hespellia (genus), Bacteroides sp. 35AE37 (species), Marvinbryantia (genus), Anaerosporobacter mobilis (species), Anaerofustis (genus), Catabacter (genus), Flavonifractor plautii (species), Proteiniphilum (genus), Roseburia faecis (species), Streptococcus sp. S16-11 (species), Bacteroides sp. 4072 (species), Alistipes shahii (species), Bacteroides intestinalis (species), Lactonifactor longoviformis (species), Bifidobacterium tsurumiense (species), Bacteroides dorei (species), Bacteroides xylanisolvens (species), Cronobacter (genus), Alloscardovia (genus), Alloscardovia omnicolens (species), Lactonifactor (genus), Catabacteriaceae (family), Adlercreutzia equolifaciens (species), Adlercreutzia (genus), Alistipes sp. EBA6-25c12 (species), Bacteroides sp. EBA5-17 (species), Oscillibacter (genus), Gordonibacter pamelaeae (species), Alistipes sp. NML05A004 (species), Parasutterella excrementihominis (species), Mitsuokella sp. DJF_RR21 (species), Butyricimonas (genus), Bifidobacterium stercoris (species), Alistipes indistinctus (species), Gordonibacter (genus), Anaerostipes hadrus (species), Klebsiella sp. B12 (species), Alistipes sp. RMA 9912 (species), Anaerosporobacter (genus), Bacteroides faecis (species), Blautia sp. Ser5 (species), Bacteroides chinchillae (species), Bilophila sp. 4_1_30 (species), Caldicoprobacteraceae (family), Enterobacter sp. UDC345 (species), Bifidobacterium biavatii (species), Peptoniphilus sp. 1-14 (species), Alistipes sp. HGB5 (species), Bacteroides sp. SLC1-38 (species), Lactobacillus sp. Akhmrol (species), Klebsiella sp. SOR89 (species), Enterococcus sp. C6 I11 (species), Pseudoflavonifractor (genus), Bacteroides sp. dnLKV9 (species), Megasphaera sp. BV3C16-1 (species), Faecalibacterium sp. canine oral taxon 147 (species), Varibaculum sp. CCUG 45114 (species), Butyricimonas sp. 214-4 (species), Anaerostipes rhamnosivorans (species), Negativicoccus sp. S5-A15 (species), [Collinsella] massiliensis (species), Corynebacterium sp. jw37 (species), Roseburia sp. 499 (species), Dialister sp. S7MSR5 (species), Anaerococcus sp. S8 87-3 (species), Finegoldia sp. S8 F7 (species), Murdochiella sp. S9 PR-10 (species), Peptoniphilus sp. S9 PR-13 (species), Bacteroides sp. J1511 (species), Corynebacterium sp. 713182/2012 (species), Rahnella sp. BSP18 (species), Intestinimonas (genus), Robinsoniella sp. KNHs210 (species), Candidatus Soleaferrea (genus), Butyricimonas faecihominis (species), Senegalimassilia (genus), Peptoniphilus sp. DNF00840 (species), Romboutsia (genus), and Coprobacter secundus (species), wherein determining the appendix-related characterization comprises determining the appendix-related characterization for the user for the appendix-related condition based on the site-specific composition features.

26. The method of claim 25, wherein determining the user microbiome features comprises determining, based on the microorganism dataset, user microbiome functional features associated with at least one of Neurodegenerative Disease, Signaling Molecules and Interaction, Xenobiotics Biodegradation and Metabolism, Ascorbate and aldarate metabolism, Huntington's disease, Inositol phosphate metabolism, Propanoate metabolism, Starch and sucrose metabolism, Caprolactam degradation, Cell motility and secretion, Valine, leucine and isoleucine degradation, Tryptophan metabolism, Type I diabetes mellitus, Phenylalanine metabolism, Selenocompound metabolism, Lysine degradation, Polycyclic aromatic hydrocarbon degradation, Glycan biosynthesis and metabolism, Renal cell carcinoma, Butanoate metabolism, Carbon fixation pathways in prokaryotes, Citrate cycle (TCA cycle), Lipopolysaccharide biosynthesis, RNA transport, Thiamine metabolism, 1,1,1-Trichloro-2,2-bis (4-chlorophenyl)ethane (DDT) degradation, Electron transfer carriers, Amyotrophic lateral sclerosis (ALS), Prion disease, Toluene degradation, alpha-Linolenic acid metabolism, [V] Defense mechanisms, [0] Post-translational modification, protein turnover, and chaperones, [R] General function prediction only, [I] Lipid transport and metabolism, [H] Coenzyme transport and metabolism, Energy Metabolism, Nervous System, Signal Transduction, Cellular Processes and Signaling, Translation, Metabolism, Cell Growth and Death, Endocrine System, Amino Acid Metabolism, Metabolism of Cofactors and Vitamins, Replication and Repair, Metabolism of Terpenoids and Polyketides, Infectious Diseases, Amino acid related enzymes, Photosynthesis, Pantothenate and CoA biosynthesis, Photosynthesis proteins, Glutamatergic synapse, Tuberculosis, Two-component system, Aminoacyl-tRNA biosynthesis, Ribosome, Other ion-coupled transporters, Terpenoid backbone biosynthesis, Cell cycle—Caulobacter, Other transporters, Base excision repair, Peptidoglycan biosynthesis, Vibrio cholerae pathogenic cycle, Limonene and pinene degradation, Secretion system, Nucleotide excision repair, Translation factors, Alanine, aspartate and glutamate metabolism, Ribosome Biogenesis, Others (KEGG3), Ribosome biogenesis in eukaryotes, Polyketide sugar unit biosynthesis, Streptomycin biosynthesis, Homologous recombination, Oxidative phosphorylation, Function unknown, Carbon fixation in photosynthetic organisms, Cytoskeleton proteins, DNA repair and recombination proteins, Inorganic ion transport and metabolism, Amino acid metabolism, Geraniol degradation, Protein export, Phenylalanine, tyrosine and tryptophan biosynthesis, Lysine biosynthesis, Ethylbenzene degradation, Transcription machinery, RNA polymerase, Biosynthesis of vancomycin group antibiotics, Mismatch repair, Naphthalene degradation, Pyrimidine metabolism, D-Glutamine and D-glutamate metabolism, Zeatin biosynthesis, K02004 (KEGG4), and K03100 (KEGG4), wherein determining the appendix-related characterization comprises determining the appendix-related characterization for the user for the appendix-related condition based on the user microbiome functional features and the site-specific composition features.

27. The method of claim 21, wherein determining the user microbiome features comprises determining, based on the microorganism dataset, site-specific composition features associated with a skin site and at least one of Pseudomonas (genus), Neisseriaceae (family), Parabacteroides distasonis (species), Prevotella (genus), Faecalibacterium prausnitzii (species), Streptococcus parasanguinis (species), Cutibacterium acnes (species), Veillonellaceae (family), Leptotrichia (genus), Phascolarctobacterium (genus), Flavobacteriaceae (family), Delftia (genus), Flavobacteriia (class), Prevotellaceae (family), Lachnospiraceae (family), Peptostreptococcaceae (family), Dorea (genus), Flavobacteriales (order), Neisseriales (order), Parabacteroides (genus), Streptococcus sp. oral taxon G63 (species), Acidaminococcaceae (family), Veillonella sp. CM60 (species), Staphylococcus sp. C912 (species), Leptotrichiaceae (family), Fusicatenibacter saccharivorans (species), Fusicatenibacter (genus), Staphylococcus sp. 334802 (species), Parabacteroides merdae (species), Collinsella aerofaciens (species), Sphingobacteriia (class), Sphingobacteriales (order), Peptoniphilus sp. 1-14 (species), Anaerobacillus (genus), Propionibacterium sp. KPL1844 (species), Methylobacterium longum (species), and Staphylococcus sp. C5116 (species), wherein determining the appendix-related characterization comprises determining the appendix-related characterization for the user for the appendix-related condition based on the site-specific composition features.

28. The method of claim 21, wherein determining the user microbiome features comprises determining, based on the microorganism dataset, site-specific composition features associated with a genital site and at least one of Gemella (genus), Veillonella atypica (species), Dialister pneumosintes (species), Lactobacillus crispatus (species), Phyllobacteriaceae (family), Aquabacterium (genus), Anaeroglobus (genus), Anaeroglobus geminatus (species), Ochrobactrum (genus), Mobiluncus curtisii (species), Actinomyces neuii (species), Anaerococcus lactolyticus (species), Lactobacillus johnsonii (species), Verrucomicrobiales (order), Verrucomicrobia (phylum), Verrucomicrobiae (class), Verrucomicrobiaceae (family), Dialister succinatiphilus (species), Atopobium sp. F0209 (species), Corynebacterium freiburgense (species), Lactobacillus sp. Akhmrol (species), Anaerococcus sp. 9401487 (species), Mesorhizobium (genus), Lactobacillus reuteri (species), Megasphaera sp. UPII 199-6 (species), Lactobacillus sp. C30An8 (species), Peptococcus sp. S9 Pr-12 (species), and Helcococcus seattlensis (species), wherein determining the appendix-related characterization comprises determining the appendix-related characterization for the user for the appendix-related condition based on the site-specific composition features.

29. The method of claim 21, wherein determining the user microbiome features comprises determining, based on the microorganism dataset, site-specific composition features associated with a mouth site and at least one of Moraxellaceae (family), Moraxella (genus), Eikenella (genus), Eikenella corrodens (species), Vagococcus (genus), Phyllobacterium (genus), Veillonella dispar (species), Sutterella wadsworthensis (species), Johnsonella ignava (species), Bacteroides acidifaciens (species), Leptotrichia hofstadii (species), Leptotrichia shahii (species), Capnocytophaga sp. AHN9756 (species), Bergeyella sp. AF14 (species), Olsenella sp. F0004 (species), Bacteroides sp. D22 (species), Phyllobacterium sp. T50 (species), Actinomyces sp. ICM47 (species), Fusobacterium sp. AS2 (species), and Leptotrichiaceae (family), wherein determining the appendix-related characterization comprises determining the appendix-related characterization for the user for the appendix-related condition based on the site-specific composition features.

30. The method of claim 21, wherein determining the user microbiome features comprises determining, based on the microorganism dataset, site-specific composition features associated with a nose site and at least one of Comamonas (genus), Peptostreptococcus (genus), Actinomyces viscosus (species), Actinomyces odontolyticus (species), Bifidobacterium (genus), Bifidobacteriaceae (family), Rhodospirillaceae (family), Bifidobacteriales (order), Roseburia intestinalis (species), Thalassospira (genus), Bifidobacterium longum (species), Aggregatibacter (genus), Streptococcus sp. 11aTha1 (species), Sutterellaceae (family), Flavobacterium (genus), Ochrobactrum (genus), Cronobacter sakazakii (species), Anaerococcus vaginalis (species), Sphingobacteriia (class), Brucellaceae (family), Sphingobacteriales (order), Akkermansia (genus), Peptoniphilus sp. gpac018A (species), Citrobacter sp. BW4 (species), Cronobacter (genus), Corynebacterium sp. jw37 (species), Staphylococcus aureus (species), Brevundimonas (genus), Caulobacteraceae (family), Caulobacterales (order), Anaerobacillus alkalidiazotrophicus (species), Anaerobacillus (genus), and Acinetobacter sp. WB22-23 (species), wherein determining the appendix-related characterization comprises determining the appendix-related characterization for the user for the appendix-related condition based on the site-specific composition features.
